(19) United States
(12) Patent Application Publication
(10) Pub. No.: US 2026/0275345 A1
(43) Pub. Date: Sep. 17, 2026

(54) COMPOSITIONS AND METHODS FOR MODULATING SIALIC ACID PRODUCTION AND TREATING HEREDITARY INCLUSION BODY MYOPATHY (HIBM)

(71) Applicant: Gradalis, Inc., Carrollton, TX (US)

(72) Inventors: Donald Rao, Dallas, TX (US); John Nemunaitis, Dallas, TX (US)

(21) Appl. No.: 19/473,318

(22) PCT Filed: Apr. 18, 2024
(86) PCT No.: PCT/US2024/025171

§ 371 Date: Oct. 7, 2025

Related U.S. Application Data
(60) Provisional application No. 63/460,433, filed on Apr. 19, 2023.

(51) Int. Cl. C12N 15/113

(52) U.S. Cl. CPC C12N 15/113; C12N 2310/14; C12N 2310/141; C12N 2310/531

(57) ABSTRACT

Compositions and methods for ameliorating the effects of hereditary inclusion body myopathy in a subject are disclosed herein. In some embodiments, the composition comprises an expression vector comprising a bifunctional short hairpin RNA (shRNA) sequence specific for knockdown of a mutant GNE in the patient.

### CROSS-REFERENCE TO RELATED APPLICATION

This application claims priority to U.S. Provisional Application No. 63/460,433, filed Apr. 19, 2023, the disclosure of which is hereby incorporated by reference in its entirety for all purposes.

### BACKGROUND

Hereditary Inclusion Body Myopathy (HIBM) (also known as GNE myopathy) is a chronic progressive skeletal muscle wasting disorder, which generally leads to complete disability before the age of 50 years. There is currently no effective therapeutic treatment for HIBM. Development of this disease is related to expression in family members of an autosomal recessive mutation of the GNE gene, which encodes the bifunctional enzyme UDP-GlcNAc 2-epimerase/ManNAc kinase (GNE/MNK). This is the rate-limiting bifunctional enzyme that catalyzes the first 2 steps of sialic acid biosynthesis. Decreased sialic acid production consequently leads to decreased sialyation of a variety of glycoproteins, including the critical muscle protein alpha-dystroglycan (α-DG). This in turn severely cripples muscle function and leads to the onset of the syndrome.

### SUMMARY OF THE DISCLOSURE

In one aspect, the disclosure provides an expression vector comprising a bifunctional small hairpin RNA (shRNA) sequence specific for knockdown of a mutant GNE, wherein the bifunctional shRNA sequence encodes a nucleic acid sequence capable of hybridizing to one or more regions of an mRNA transcript encoding the mutant GNE to inhibit the expression of the mutant GNE via RNA interference, wherein the bifunctional shRNA comprises a first stem-loop structure that comprises an siRNA component and a second stem-loop structure that comprises a miRNA component.

In some embodiments, the one or more regions of the mRNA transcript encoding the mutant GNE are selected from nucleotides 526-528, 1714-1716, and 2134-2136 of SEQ ID NO:16.

In some embodiments, the siRNA component functions in a cleavage-dependent manner and the miRNA component functions in a cleavage-independent manner.

In some embodiments, the bifunctional shRNA sequence is operably linked to a promoter. In particular embodiments, the promoter is a CMV mammalian promoter.

In some embodiments, the bifunctional shRNA sequence comprises a sequence having at least 90% identity to any one of SEQ ID NOS:3-6.

In some embodiments, the bifunctional shRNA sequence comprises a sequence having at least 90% identity to any one of SEQ ID NOS:31 and 32.

In another aspect, the disclosure provides a composition comprising the expression vector described herein and a wild-type GNE-encoding nucleic acid sequence. In some embodiments, the wild-type GNE-encoding nucleic acid sequence comprises SEQ ID NO:1.

In some embodiments, the wild-type GNE-encoding nucleic acid sequence comprises SEQ ID NO:27.

In some embodiments, the wild-type GNE-encoding nucleic acid sequence and the bifunctional shRNA sequence are provided in one or more liposomes or lipid nanoparticles.

In some embodiments, the wild-type GNE-encoding nucleic acid sequence and/or the bifunctional shRNA sequence comprises a promoter operably connected to the wild-type GNE-encoding nucleic acid sequence and/or the bifunctional shRNA sequence. In some embodiments, the promoter is the CMV promoter.

In some embodiments, the wild-type GNE-encoding nucleic acid sequence and/or the bifunctional shRNA sequence is disposed within or is connected to a lipososome or a lipid nanoparticle. In particular embodiments, the liposome or the lipid nanoparticle comprises one or more agents capable of recognizing and binding to a muscle cell or a component thereof.

In another aspect, the disclosure provides a method for ameliorating the effects of hereditary inclusion body myopathy, which comprises the steps of: identifying a human subject with hereditary inclusion body myopathy; and providing the human subject with effective amounts of a wild-type GNE-encoding nucleic acid sequence and a bifunctional shRNA sequence that knocks down the expression of a mutant GNE in the human subject by administration at a location with hereditary inclusion body myopathy, wherein the wild-type GNE-encoding nucleic acid sequence comprises SEQ ID NO:1 and the bifunctional shRNA sequence comprises a sequence having at least 90% identity to any one of SEQ ID NOS:3-6.

In another aspect, the disclosure provides a method for ameliorating the effects of hereditary inclusion body myopathy, which comprises the steps of: identifying a human subject with hereditary inclusion body myopathy; and providing the human subject with effective amounts of a wild-type GNE-encoding nucleic acid sequence and a bifunctional shRNA sequence that knocks down the expression of a mutant GNE in the human subject by administration at a location with hereditary inclusion body myopathy or systemically, wherein the wild-type GNE-encoding nucleic acid sequence comprises SEQ ID NO:27 and the bifunctional shRNA sequence comprises a sequence having at least 90% identity to any one of SEQ ID NOS:31 and 32.

In some embodiments, the administration is by systemic infusion. In particular embodiments, the systemic infusion delivers the wild-type GNE-encoding nucleic acid sequence and the bifunctional shRNA sequence into muscles.

In some embodiments, the administration is intramuscular injection.

In some embodiments, the wild-type GNE-encoding nucleic acid sequence and the bifunctional shRNA sequence are provided in one or more liposomes or lipid nanoparticles.

In some embodiments, the administration is via intramuscular administration to human muscle cells.

In some embodiments, the wild-type GNE-encoding nucleic acid sequence comprises a promoter operably connected to the wild-type GNE-encoding nucleic acid sequence. In some embodiments, the promoter is the CMV promoter.

In some embodiments, the wild-type GNE-encoding nucleic acid sequence and/or the bifunctional shRNA sequence is disposed within or is connected to the liposome or the lipid nanoparticle. In some embodiments, the liposome or the lipid nanoparticle comprises one or more agents capable of recognizing and binding to a muscle cell or a component thereof.

In another aspect, the disclosure provides a method for modulating the production of sialic acid in a human, which comprises the steps of: providing a human subject in need of treatment of a hereditary inclusion body myopathy; providing a human wild-type GNE-encoding nucleic acid sequence and a bifunctional shRNA sequence that knocks down the expression of a mutant GNE in the human subject by administration at a location with hereditary inclusion body myopathy, wherein the wild-type GNE-encoding nucleic acid sequence comprises SEQ ID NO:1 and the bifunctional shRNA sequence comprises a sequence having at least 90% identity to any one of SEQ ID NOS:3-6.

In another aspect, the disclosure provides a method for modulating the production of sialic acid in a human, which comprises the steps of: providing a human subject in need of treatment of a hereditary inclusion body myopathy; providing a human wild-type GNE-encoding nucleic acid sequence and a bifunctional shRNA sequence that knocks down the expression of a mutant GNE in the human subject by administration at a location with hereditary inclusion body myopathy, wherein the wild-type GNE-encoding nucleic acid sequence comprises SEQ ID NO:27 and the bifunctional shRNA sequence comprises a sequence having at least 90% identity to any one of SEQ ID NOS:31 and 32.

In some embodiments, the wild-type GNE-encoding nucleic acid sequence and the bifunctional shRNA sequence are provided in one or more liposomes or lipid nanoparticles.

In some embodiments, the administration is via intramuscular administration to human muscle cells.

In some embodiments, the wild-type GNE-encoding nucleic acid sequence comprises a promoter operably connected to the wild-type GNE-encoding nucleic acid sequence. In some embodiments, the promoter is the CMV promoter.

In some embodiments, the wild-type GNE-encoding nucleic acid sequence and/or the bifunctional shRNA is disposed within or is connected to the liposome or the lipid nanoparticle. In some embodiments, the liposome or the lipid nanoparticle comprises one or more agents capable of recognizing and binding to a muscle cell or a component thereof.

In another aspect, the disclosure provides a method for expressing a wild-type GNE and a bifunctional shRNA that knocks down the expression of a mutant GNE in a human subject with the mutant GNE, comprising: administering a wild-type GNE-encoding sequence and a bifunctional shRNA sequence at a location in a muscle with hereditary inclusion body myopathy, and wherein the wild-type GNE-encoding nucleic acid sequence comprises SEQ ID NO:1 and the bifunctional shRNA sequence comprises a sequence having at least 90% identity to any one of SEQ ID NOS:3-6.

In another aspect, the disclosure provides a method for expressing a wild-type GNE and a bifunctional shRNA that knocks down the expression of a mutant GNE in a human subject with the mutant GNE, comprising: administering a wild-type GNE-encoding sequence and a bifunctional shRNA sequence at a location in a muscle with hereditary inclusion body myopathy, and wherein the wild-type GNE-encoding nucleic acid sequence comprises SEQ ID NO:27 and the bifunctional shRNA sequence comprises a sequence having at least 90% identity to any one of SEQ ID NOS:31 and 32.

In some embodiments, the wild-type GNE-encoding nucleic acid sequence and the bifunctional shRNA sequence are provided in one or more liposomes or lipid nanoparticles.

In some embodiments, the administering is via intramuscular administration to human muscle cells.

In some embodiments, the wild-type GNE-encoding nucleic acid sequence comprises a promoter operably connected to the wild-type GNE-encoding nucleic acid sequence. In particular embodiments, the promoter is the CMV promoter.

In some embodiments, the wild-type GNE-encoding nucleic acid sequence and/or the bifunctional shRNA is disposed within or is connected to the liposome or the lipid nanoparticle. In some embodiments, the liposome or the lipid nanoparticle comprises one or more agents capable of recognizing and binding to a muscle cell or a component thereof.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1: plasmid map of pGBI-1000.

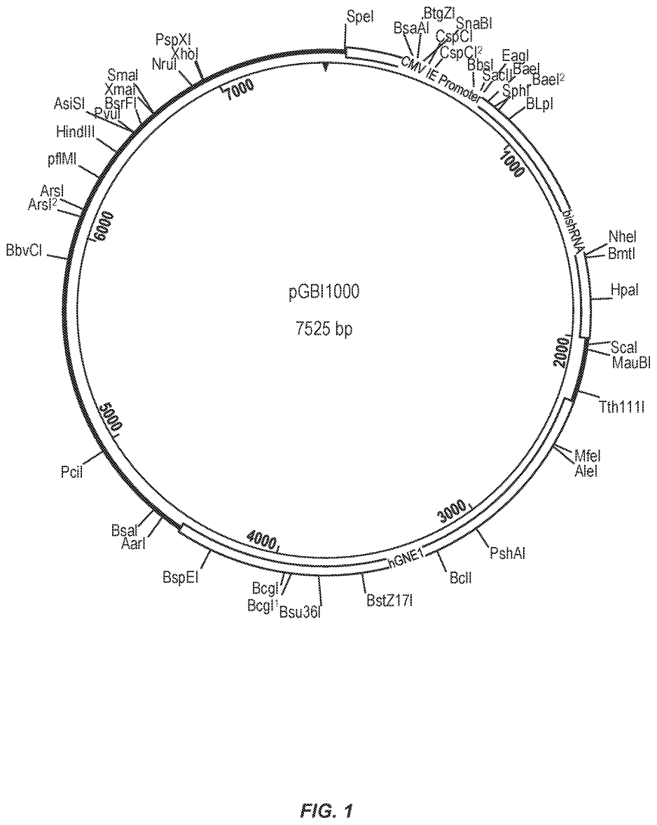

FIG. 2: plasmid map of pGBI-1005.

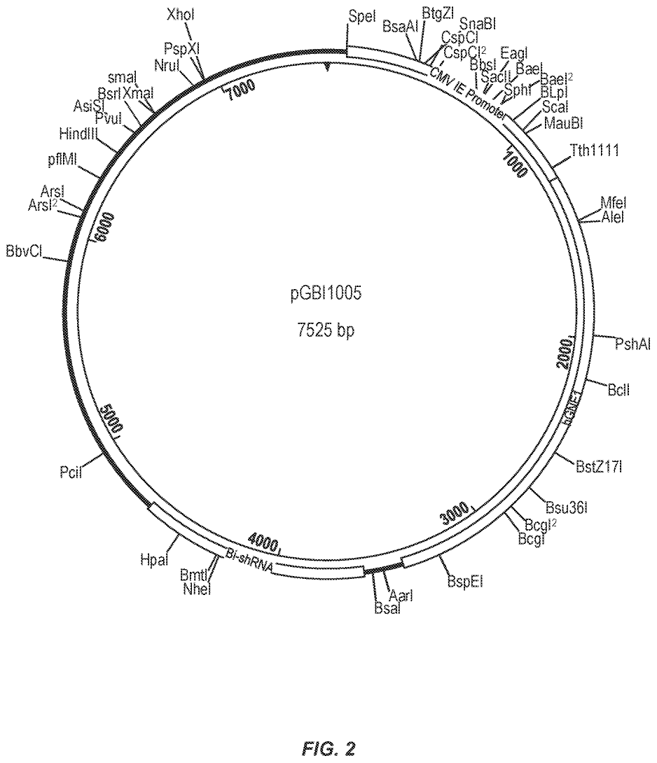

FIG. 3: plasmid map of pGBI-1001 (SEQ ID NO:20).

FIG. 4: plasmid map of pGBI-1006 (SEQ ID NO:21).

FIG. 5: plasmid map of pGBI-1013 (SEQ ID NO:22).

FIG. 6: plasmid map of pGBI-1014 (SEQ ID NO:23).

FIG. 7: a schematic illustration of RT-PCR based restriction fragment length polymorphism (RFLP) assay to assess M743T mutant mRNA knockdown without affecting the wild-type mRNA.

FIG. 8: Three reverse primers used in RFLP.

FIG. 9: Data from RFLP assay by co-transfecting hGNE2 M743T mutation expression plasmid either with hGNE2 wild-type expression plasmid or with four designed dual-function expression plasmids.

FIG. 10: plasmid map of pGBI-1011 (SEQ ID NO:24).

FIG. 11: plasmid map of pGBI-1012 (SEQ ID NO:25).

FIG. 12: Western blot analysis of total protein isolated from cells transfected with one of four dual-function plasmids (#1-#4, SEQ ID NOS:20-23) with either strep tagged wild-type hGNE2 expression plasmid or strep tagged M743T mutant hGNE2 expression plasmid.

FIG. 13: plasmid map of the Doggybone DNA plasmid (SEQ ID NO:26).

FIG. 14: plasmid map of vector expressing wild-type hGNE2 in pUMVC3 backbone (SEQ ID NO:29).

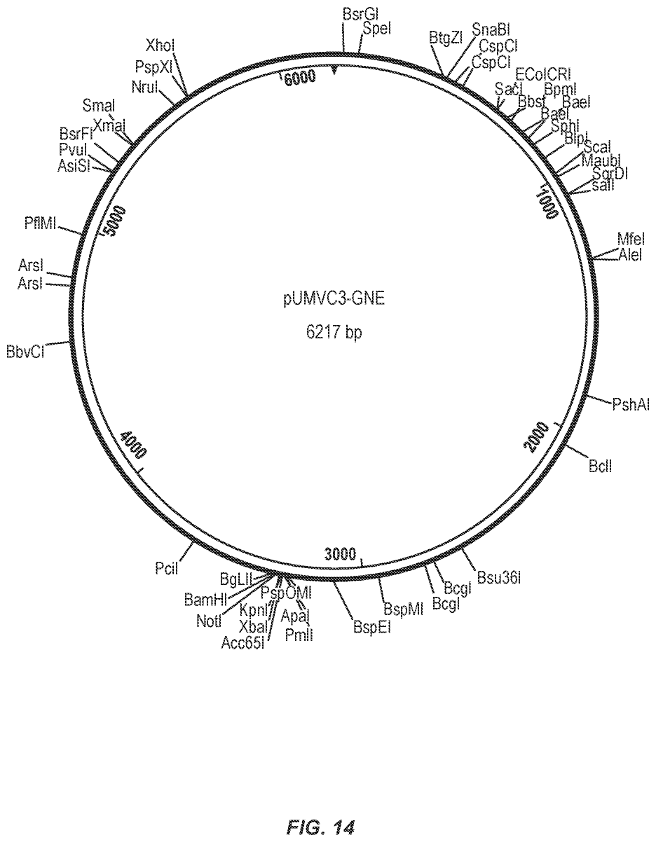

FIG. 15: plasmid map of vector expressing mutant hGNE2 M743T in pUMVC3 backbone (SEQ ID NO:30).

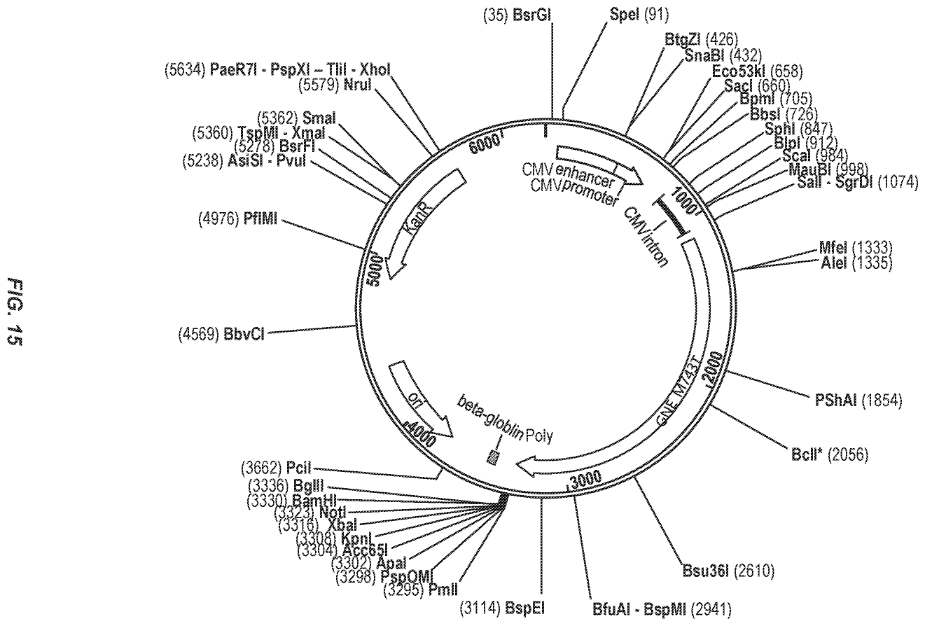

### DETAILED DESCRIPTION

Disclosed herein, in certain embodiments, are expression vectors comprising a bifunctional short hairpin RNA (shRNA) sequence specific for knockdown of a mutant GNE. The bifunctional shRNA sequence encodes a nucleic acid sequence capable of hybridizing to one or more regions of an mRNA transcript encoding the mutant GNE to inhibit the expression of the mutant GNE via RNA interference. The bifunctional shRNA comprises a first stem-loop structure that comprises an siRNA component and a second stem-loop structure that comprises a miRNA component.

A mutant GNE can comprise at least one mutation relative to the sequence of a wild-type GNE (e.g., hGNE1 having SEQ ID NO:2; hGNE2 having SEQ ID NO:28). In some embodiments, a mutant GNE is a mutant hGNE1 and comprises at least one mutation selected from D176V, V572L, and M712T. In some embodiments, a mutant GNE is a mutant hGNE1 and comprises the mutation D176V. In some embodiments, a mutant GNE is a mutant hGNE1 and comprises the mutation V572L. In some embodiments, a mutant GNE is a mutant hGNE1 and comprises the mutation M712T. In some embodiments, a mutant GNE is a mutant hGNE1 and comprises all three mutations D176V, V572L, and M712T. In some embodiments, a mutant GNE is a mutant hGNE2 and comprises at least one mutation selected from D207V, V603L, and M743T. In some embodiments, a mutant GNE is a mutant hGNE2 and comprises the mutation D207V. In some embodiments, a mutant GNE is a mutant hGNE2 and comprises the mutation V603L. In some embodiments, a mutant GNE is a mutant hGNE2 and comprises the mutation M743T. In some embodiments, a mutant GNE is a mutant hGNE2 and comprises all three mutations D207V, V603L, and M743T.

In some embodiments, a bifunctional shRNA can bind to one or more regions of the mRNA transcript encoding a mutant GNE (e.g., a mutant hGNE1 comprising mutations D176V, V572L, and M712T) selected from nucleotides 526-528, 1714-1716, and 2134-2136 of SEQ ID NO:16.

In some embodiments, a bifunctional shRNA can bind to one or more regions of the mRNA transcript encoding a mutant GNE (e.g., a mutant hGNE2 comprising mutations D207V, V603L, and M743T),

The disclosure also provides compositions that comprise an expression vector having a bifunctional shRNA sequence specific for knockdown of a mutant GNE (e.g., a mutant hGNE1 comprising at least one mutation selected from D176V, V572L, and M712T). The disclosure also provides compositions that comprise an expression vector having a bifunctional shRNA sequence specific for knockdown of a mutant GNE (e.g., a mutant hGNE2 comprising at least one mutation selected from D207V, V603L, and M743T). In addition, the composition can further comprise a wild-type GNE-encoding nucleic acid sequence. In certain embodiments, the wild-type GNE-encoding nucleic acid sequence can be in the same expression vector as the bifunctional shRNA. In other embodiments, a composition can comprise a first expression vector comprising a bifunctional shRNA sequence specific for knockdown of a mutant GNE (e.g., a mutant hGNE1 or a mutant hGNE2), and a second expression vector comprising a wild-type GNE-encoding nucleic acid sequence.

As described in detail further here, the wild-type GNE-encoding nucleic acid sequence and the bifunctional shRNA sequence are provided in one or more liposomes or lipid nanoparticles. In certain embodiments, the liposome or the lipid nanoparticle can comprise one or more agents capable of recognizing and binding to a muscle cell or a component thereof, such that the wild-type GNE-encoding nucleic acid sequence and the bifunctional shRNA sequence can be targeted to a muscular region of a patient in need. In addition, a muscle specific promotor that expresses the protein in muscle tissue can be used (e.g., muscle creatine kinase promotor). See V. V. Skopenkova et al., “Muscle-Specific Promoters for Gene Therapy” Acta Naturae, 2021 January-March; 13(1): 47-58, incorporated herein by reference. As taught therein, several series of small MCK expression cassettes for adenoviral vectors were developed and tested such as the construct CK6, consisting of an enhancer (206 bp) and a proximal promoter (358 bp), which ensures high muscle specificity.

The expression vectors and compositions described herein can be used in methods for ameliorating the effects of hereditary inclusion body myopathy in a subject with hereditary inclusion body myopathy. The expression vectors and compositions described herein can also be used in methods for modulating the production of sialic acid in a human with hereditary inclusion body myopathy. The expression vectors and compositions described herein can also be used in methods for expressing a wild-type GNE and a bifunctional shRNA that knocks down the expression of a mutant GNE in a human subject with hereditary inclusion body myopathy.

### INTRODUCTION

GNE (UDP-N-acetylglucosamine 2-epimerase/N-acetylmannosamine kinase) myopathy, formerly known Inclusion Body Myopathy 2, Quadriceps Sparing Myopathy or Hereditary Inclusion Body Myopathy is an ultra-rare autosomal recessive non-inflammatory muscle disease characterized by early adult onset and progressive debilitating muscle dysfunction transmitted through a variety of mutations of the GNE gene [1, 2]. The M743T mutation is the most common variant globally and is of higher incidence in people of Persian Jewish decent [2]. GNE wildtype (wt) protein is the rate limiting enzyme that catalyzes the first two steps of the biosynthesis of sialic acid [3]. It is our belief that reestablishment of GNEwt function by providing GNEwt protein via intramuscular plasmid delivery of expressive GNEwt gene while concurrently diminishing adverse activity of M743T mutated GNE protein with concurrent knockdown with bi-shRNA-hGNE2-M743T will diminish muscle deterioration related to M743T GNE induced myopathy.

Sialic acids are typically found as the terminal sugars on glycoconjugates, where they play pivotal roles in cellular signaling events [4]. GNE myopathy-associated GNE mutations have been shown to reduce sialic acid production which is essential for proper folding, stabilization, and function of skeletal muscle glycoproteins [5-8]. GNE mutations resulting in hyposialyation of muscle glycoproteins appear to contribute to myofibrillar degeneration and loss of normal muscle function [9]. Thus, most in the field conclude that impaired GNE function, not lack of expression, is the key pathogenic factor in GNE myopathy [10, 11]. Indeed, Penner et al. [12] characterized several different GNE mutations and demonstrated altered activity of GNE enzyme related to mutation correlated with varying degrees of severity, as assessed by downstream enzyme kinetics of ManNAc phosphorylation using a radiolabeled phosphate assay.

Recently, the mutant hGNE2 variant protein M743T was shown to have significantly reduced enzymatic activity when compared to GNEwt. This effect was related to a single point mutation leading to a substitution of threonine from methionine at position 743 and relates to a change in oligomeric state and possibly protein folding of hGNE2 [3, 13]. Bennmann et al [14] found that the M743T variant had a 3-fold increase in O-GlcNAcylation compared to GNEwt. Moreover, the half-life of the M743T variant was more than 2-fold longer than the half-life of GNEwt protein. Thereby, increasing concentration of M743T, putting dominating control of muscle function capacity in the hands of dysfunctional hGNE2 M743T protein, which clinically has demonstrated results leading to severe myopathy at midlife age onset.

A GNE-wt-DNA vector using human GNE cDNA and the pUMVC3 expression vector was constructed and it was demonstrated transgene expression of GNE mRNA and GNE protein in correlation with subsequent increased production of sialic acid in CHO-Lec3 cells in vitro [15]. The GNE expression vector was also complexed with a cationic liposome, composed of 1,2-dioleoyl-3-trimethylammonium-propane (DOTAP) and cholesterol (GNE-Lipoplex) and dose related safety was demonstrated in BALB/c mice with IM and IV injection [9, 16]. These results demonstrated correlation of the pUMVC-GNE transfection with GNE transgene and protein expression at the desired target site, murine muscle, and provided no evidence in murine model of toxic effect at dose levels ≤40 g via IM or IV delivery. Safety and improvement were also demonstrated in local muscle strength correlating with enhancement of rGNE muscle transgene expression and consequent increase of cell surface sialic acid following IM injection of GNE-Lipoplex in a single patient with late stage GNE myopathy [17]. Furthermore, muscle uptake and expression, safety and clinical benefit were demonstrated in the same patient following intravenous infusion of GNE gene lipoplex [18].

The disclosure provides a novel upgraded GNE plasmid design to achieve concurrent knockdown of mutant GNE (M743T) with a bi-shRNAi insert engineered downstream from the CMV-GNEwt plasmid insert.

### GNE Nomenclature

As described by Huizing et al. (Neuromuscul Disord. 2014 May; 24(5): 387-389), after initial discovery of GNE gene defects to be causative for GNE myopathy, eight different GNE mRNA splice variants were identified, encoding (at least theoretically) eight protein isoforms. As described herein, human GNE 1 (hGNE1) is the originally described GNE protein which covers 722 amino acids as shown in SEQ ID NO:2. Human GNE 2 (hGNE2) contains an additional N-terminal sequence relative to hGNE1 and covers 753 amino acids as shown in SEQ ID NO:28. The table below extracted from Huizing et al. provides a summary of hGNE2 isoform and hGNE1 isoform, as well as their corresponding amino acid mutations.

TABLE
from Huizing et al. (Neuromuscul Disord. 2014 May; 24(5): 387-389)

| New Nomenclature¹ | | | Previous Nomenclature | | | |
|---|---|---|---|---|---|---|
| hGNE2 isoform | mRNA transcript | Exon | hGNE1 isoform | mRNA transcript | Exon | Ethnicity² |
| GenBank hGNE2 | GenBank Variant 1 | GenBank GNE | GenBank hGNE1 | GenBank | | |
| NP_001121699 | NM_001128227 | gDNA | NP_005467 | Variant 2 | | |
| | | NC_000009 | | NM_005476 | | |
| ENSEMBL GNE- | ENSEMBL GNE-003 ENST | ENSEMBL GNE | ENSEMBL | ENSEMBL | | |
| 003 | 00000396594 | gDNA | GNE-001 | GNE-001 | | |
| ENSP | | ENSG | ENSP | ENST | | |
| 00000379839 | | 00000159921 | 00000367134 | 00000377902 | | |
| p.C44S | c.131G > C | 3 | p.C13S | c.38G > C | 2 | Japanese |
| p.D207V | c.620A > T | 4 | p.D176V | c.527A > T | 3 | Japanese |
| p.V603L | c.1807G > C | 11 | p.V572L | c.1714G > C | 10 | Japanese |
| p.I618T | c.1853T > C | 11 | p.1587T | c.1760T > C | 10 | Cajun, Roma |
| | | | | | | Gypsies |
| p.V727M | c.2179G > A | 13 | p.V696M | c.2086G > A | 12 | Indian |
| p.M743T | c.2228T > C | 13 | p.M712T | c.2135T > C | 12 | Middle Eastern |

¹Nomenclature according to universally adapted gene/protein nomenclature rules.
²Ethnicity in which the variant is mostly reported.

### Definitions

Unless defined otherwise, all technical and scientific terms used herein have the same meaning as is commonly understood by one of skill in the art to which the claimed subject matter belongs.

As used herein, ranges and amounts can be expressed as “about” a particular value or range. About also includes the exact amount. Hence “about 5 μg” means “about 5 μg” and also “5 μg.” Generally, the term “about” includes an amount that would be expected to be within experimental error. In some embodiments, “about” refers to the number or value recited, “+” or “−” 20%, 10%, or 5% of the number or value.

As used herein the term “bi-functional” refers to a shRNA having two mechanistic pathways of action, that of the siRNA and that of the miRNA. The term “traditional” shRNA refers to a DNA transcription derived RNA acting by the siRNA mechanism of action. The term “doublet” shRNA refers to two shRNAs sets, each acting against the expression of two different genes but in the “traditional” siRNA mode.

As used herein, the terms “GNE-encoding nucleic acid sequence,” “wild-type GNE-encoding sequence,” “GNE-encoding sequence,” and similar terms refer to a nucleic acid sequence that encodes the wild-type bifunctional enzyme UDP-GlcNAc 2-epimerase/ManNAc kinase (GNE/MNK). There are two isoforms of wild-type human GNE: hGNE1 (mRNA transcript of SEQ ID NO:1; protein sequence of SEQ ID NO:2), and hGNE2 (mRNA transcript of SEQ ID NO:27; protein sequence of SEQ ID NO:28). A GNE-encoding sequence may only include a nucleic acid sequence that encodes the wild-type form of GNE (e.g., hGNE1 or hGNE2). Alternatively, the GNE-encoding sequence may comprise the nucleic acid sequence that encodes the wild-type form of GNE, along with other transcriptional control elements, such as a promoter, termination sequence, and/or other elements.

The terms “GNE-encoding nucleic acid sequence,” “wild-type GNE-encoding sequence,” “GNE-encoding sequence,” and similar terms are further meant to include a nucleic acid sequence which, by virtue of the degeneracy of the genetic code, may not be identical with that shown in any of the sequences shown herein, but which still encodes the amino acid sequence of the wild-type GNE (e.g., hGNE1 of SEQ ID NO:2, or hGNE2 of SEQ ID NO:28), or a modified nucleic acid sequence that encodes a different amino acid sequence, provided that the resulting GNE protein retains substantially the same (or even an improved) activity of the wild-type GNE protein. A non-limiting example of such a modified GNE protein includes the GNE isoform R266Q. That is, modifications to a GNE-encoding sequence that alter the amino acid sequence of the wild-type GNE protein in such a way that one amino acid is replaced with a similar amino acid are encompassed by the present invention, as well as other modifications which do not substantially negatively affect GNE activity because the change (whether it be substitution, deletion or insertion) does not negatively affect the active site of the GNE protein.

According to certain embodiments of the invention, the GNE-encoding sequence may be disposed in or connected to an appropriate carrier or delivery vehicle. Various strategies may be employed to deliver the GNE-encoding sequences described herein into target cells, including the use of lipid carriers (lipid nanoparticles), viral vectors, biodegradable polymers, polymer microspheres, and various conjugate systems and related cytofectins.

The terms “effective amount” or “therapeutically effective amount,” as used herein, refer to a sufficient amount of an agent or a compound being administered which will relieve to some extent one or more of the symptoms of the disease or condition being treated or prevent the onset or recurrence of the one or more symptoms of the disease or condition being treated. In some embodiments, the result is reduction and/or alleviation of the signs, symptoms, or causes of a disease, or any other desired alteration of a biological system. For example, an “effective amount” for therapeutic uses is the amount of the autologous tumor cell vaccine required to provide a clinically significant decrease in disease symptoms without undue adverse side effects. In another example, an “effective amount” for therapeutic uses is the amount of the autologous tumor cell vaccine as disclosed herein required to prevent a recurrence of disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, is an amount effective to achieve a desired effect or therapeutic improvement without undue adverse side effects. It is understood that, in some embodiments, “an effective amount” or “a therapeutically effective amount” varies from subject to subject, due to variation in metabolism of the autologous tumor cell vaccine, age, weight, general condition of the subject, the condition being treated, the severity of the condition being treated, and the judgment of the prescribing physician. The phrase “therapeutically effective amount” of a wild-type GNE-encoding nucleic acid sequence refers to a sufficient amount of the sequence to express sufficient levels of wild-type GNE, at a reasonable benefit-to-risk ratio, to increase sialic acid production in the targeted cells and/or to otherwise treat, prevent, and/or ameliorate the effects of HIBM in a patient. The phrase “therapeutically effective amount” of a bifunctional shRNA refers to a sufficient amount of the sequence to knockdown the expression of a mutant GNE gene in the targeted cells of the patient and/or to otherwise treat, prevent, and/or ameliorate the effects of HIBM in the patient. It will be understood, however, that the total daily usage of the wild-type GNE-encoding nucleic acid sequence, the bifunctional shRNA sequence, and related compositions will be decided by the attending physician, within the scope of sound medical judgment.

As used herein, the terms “subject,” “individual,” and “patient” are used interchangeably. None of the terms are to be interpreted as requiring the supervision of a medical professional (e.g., a doctor, nurse, physician's assistant, orderly, hospice worker). As used herein, the subject is any animal, including mammals (e.g., a human or non-human animal) and non-mammals. In one embodiment of the methods and autologous tumor cell vaccines provided herein, the mammal is a human.

As used herein, the terms “treat,” “treating,” or “treatment,” and other grammatical equivalents, including, but not limited to, alleviating, abating, or ameliorating one or more symptoms of a disease or condition, ameliorating, preventing or reducing the appearance, severity, or frequency of one or more additional symptoms of a disease or condition, ameliorating or preventing the underlying metabolic causes of one or more symptoms of a disease or condition, inhibiting the disease or condition, such as, for example, arresting the development of the disease or condition, relieving the disease or condition, causing regression of the disease or condition, relieving a condition caused by the disease or condition, preventing recurrence or prophylactically treating recurrence of the disease or condition, or inhibiting the symptoms of the disease or condition either prophylactically and/or therapeutically. In a non-limiting example, for prophylactic benefit, an autologous tumor cell vaccine composition disclosed herein is administered to an individual at risk of developing a particular disease or condition, predisposed to developing a particular disease or condition, or to an individual previously suffering from and treated for the disease or condition. In some embodiments, the disease or condition is hereditary inclusion body myopathy (HIBM).

As used herein, the term “prevention” means a prophylactic treatment performed before the subject suffers from a disease or the disease previously diagnosed is deteriorated, thereby enabling the subject to avoid, prevent or reduce the likelihood of the symptoms or related diseases of the disease. The subject may be a subject with an increased risk of developing a disease or a disease previously diagnosed to be deteriorated.

As used herein, the term “transfection” refers to the introduction of foreign DNA into eukaryotic cells. In some embodiments, transfection is accomplished by any suitable means, such as for example, calcium phosphate-DNA co-precipitation, DEAE-dextran-mediated transfection, polybrene-mediated transfection, electroporation, microinjection, liposome fusion, lipofection, protoplast fusion, retroviral infection, or biolistics.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. In some embodiments, nucleic acid molecules are composed of monomers that are naturally-occurring nucleotides (such as DNA and RNA), or analogs of naturally-occurring nucleotides (e.g., α-enantiomeric forms of naturally-occurring nucleotides), or a combination of both. In some embodiments, modified nucleotides have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, in some embodiments, the entire sugar moiety is replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. In some embodiments, nucleic acid monomers are linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. In some embodiments, the term “nucleic acid” or “nucleic acid molecule” also includes so-called “peptide nucleic acids,” which comprise naturally-occurring or modified nucleic acid bases attached to a polyamide backbone. In some embodiments, nucleic acids are single stranded or double stranded.

As used herein, the term “expression vector” refers to nucleic acid molecules encoding a gene that is expressed in a host cell. In some embodiments, an expression vector comprises a transcription promoter, a gene, and a transcription terminator. In some embodiments, gene expression is placed under the control of a promoter, and such a gene is said to be “operably linked to” the promoter. In some embodiments, a regulatory element and a core promoter are operably linked if the regulatory element modulates the activity of the core promoter. As used herein, the term “promoter” refers to any DNA sequence which, when associated with a structural gene in a host cell, increases, for that structural gene, one or more of 1) transcription, 2) translation or 3) mRNA stability, compared to transcription, translation or mRNA stability (longer half-life of mRNA) in the absence of the promoter sequence, under appropriate growth conditions.

### Liposomes and Lipid Nanoparticles

The use of lipids, liposomes, exosomes, proteins or other particle forming compositions are a delivery vehicle for the GNE-encoding sequences, as well as the bifunctional shRNA sequence, described herein. Liposomes are attractive carriers insofar as they protect biological molecules, such as the GNE-encoding sequences and the bifunctional shRNA sequence described herein, from degradation while improving cellular uptake. One of the most commonly used classes of liposome formulations for delivering polyanions (e.g., DNA) is that which contains cationic lipids. Other delivery systems, e.g., viral and non-viral delivery systems (e.g., cationic polymers, poly(L-lysine), polysaccharides, and poly(ethylenimine)s) may also be considered and used (see, e.g., Sung and Kim, Biomater Res. 2019 Mar. 12:23:8).

Lipid aggregates may be formed with macromolecules using cationic lipids alone or including other lipids and amphiphiles, such as phosphatidylethanolamine. It is well-known in the art that both the composition of the lipid formulation, as well as its method of preparation, have an effect on the structure and size of the resultant anionic macromolecule-cationic lipid aggregate. These factors can be modulated to optimize delivery of polyanions to specific cell types in vitro and in vivo.

The use of cationic lipids for cellular delivery of the GNE-encoding nucleic acid sequence and the bifunctional shRNA sequence described herein has several advantages. The encapsulation of anionic compositions using cationic lipids is essentially quantitative due to electrostatic interaction. In addition, it is believed that the cationic lipids interact with the negatively charged cell membranes, thereby initiating cellular membrane transport.

Experiments have shown that plasmid DNA may be encapsulated in small particles, which generally consist of a single plasmid encapsulated within a bilayer lipid vesicle (Wheeler, et al., 1999, Gene Therapy 6, 271-281). These particles often contain the fusogenic lipid such as dioleoylphosphatidylethanolamine (DOPE), low levels of a cationic lipid, and can be stabilized in aqueous media by the presence of a poly(ethylene glycol) (PEG) coating.

These lipid particles have systemic applications, as they exhibit extended circulation lifetimes following intravenous (i.v.) injection, can accumulate preferentially in various tissues and organs due to the enhanced vascular permeability in such regions, and can be designed to escape the lyosomic pathway of endocytosis by disruption of endosomal membranes. These properties can be useful in delivering biologically active molecules, such as GNE-encoding sequences, to various cell types for experimental and therapeutic applications, such as to muscle tissue cells. Various lipid nucleic acid particles and methods of preparation thereof are described in U.S. Patent Application Publication Nos. 2008-0020058, 2003-0077829, 2003-0108886, 2006-0051405, 2006-0083780, 2003-0104044, 2006-0051405, 2004-0142025, 2006-00837880, 2005-0064595, 2005-0175682, 2005-0118253, 2005-0255153 and 2005-0008689; and U.S. Pat. Nos. 5,885,613; 6,586,001; 6,858,225; 6,858,224; 6,815,432; 6,586,410; 6,534,484; and 6,287,591, all of which are incorporated herein by reference in their entirety.

The invention provides that the GNE-encoding sequence, the bifunctional shRNA sequence, and the associated delivery vehicles used therewith, may be targeted towards specific cell types, for example, muscle cells, muscle tissue, and the like. For example, the liposomal nanoparticles can be directed to bind to cell surfaces by a number of specific interactions. This binding facilitates the uptake of the DNA into the cell by one of several well understood cell entry pathways. Rapid sequestration of the nanoparticles (e.g., liposomes) by these interactions reduces their time in the peripheral circulation, thereby decreasing the likelihood of degradation and nonspecific uptake. General targeting agents include, but are not limited to, transferrin (Trf) which binds to the transferrin receptor (TrfR) on a cell surface—or using an antibody (or a derivative thereof) that binds to the TrfR on the cell surface. Muscle has a relatively high proportion of TrfR on its cell surfaces. Another target for sequestration is the epidermal growth factor receptor (EGFR), which is prevalent on the surface of muscle cells and other epitheleoid cell types. Erbitux (an EGFR monoclonal antibody approved for human use) is an exemplary agent for EGFR-targeting, which may also be used to decorate the liposomal nanoparticles described herein. Additional targeting moieties can be, but are not limited to, lectins or small molecules (peptides or carbohydrates) which recognize and bind to specific targets found only on (or are more restricted to) muscle cells. The advantage of smaller (and possibly higher affinity) molecules is that they could be present at a higher density on the surface of the nanoparticles employed.

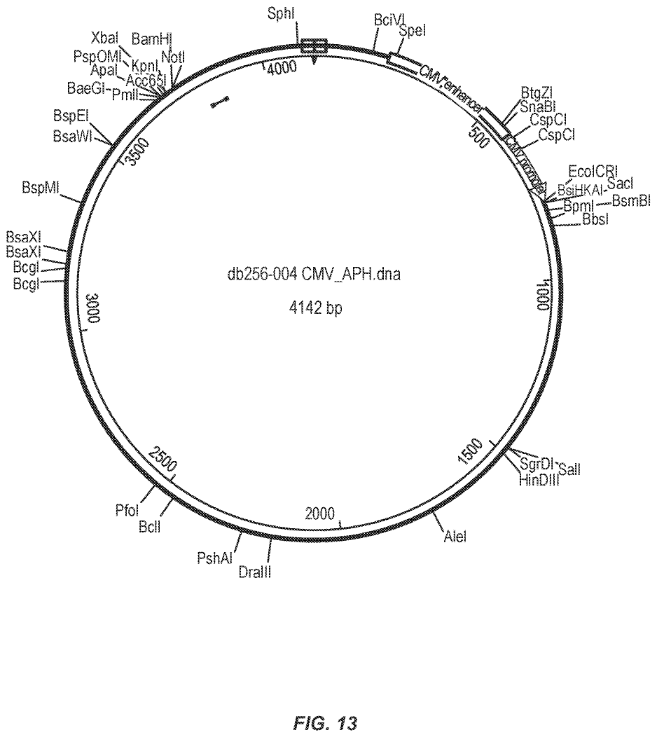

In one aspect, the particle comprises a Doggybone (dbDNA™) DNA. Named after its schematic structure, dbDNA™ is a minimal, linear, double stranded and covalently closed DNA construct. The Doggybone platform is available from the commercial supplier Touchlight. FIG. 13 shows a schematic of the Doggybone DNA vector and the sequence of an exemplary vector is shown in SEQ ID NO:26.

### Administration

The GNE-encoding sequence and the bifunctional shRNA sequence described herein, which can be delivered to a system in connection with an appropriate delivery vehicle (such as a liposome or lipid nanoparticle), may be administered to a system using any of various well-known techniques. For example, in the case of a mammal, the GNE-encoding sequence and the bifunctional shRNA sequence may be administered to a mammal via parenteral injection. The term “parenteral,” as used herein, includes subcutaneous, intracutaneous, intravenous, intramuscular, intraarticular, intraarterial, intrasynovial, or infusion techniques. In particular embodiments, the GNE-encoding sequence and the bifunctional shRNA sequence can be administered via intramuscular injection. In some embodiments, the GNE-encoding sequence and the bifunctional shRNA sequence can be administered via systemic infusion. In particular embodiments, the systemic infusion delivers the GNE-encoding sequence and the bifunctional shRNA sequence into muscles.

The GNE-encoding sequence, the bifunctional shRNA sequence, and related compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated composition or its delivery form. For example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid may be used in the preparation of injectables.

According to certain embodiments, a Plasma-Lyte® carrier may be employed and used to deliver the GNE-encoding sequence and the bifunctional shRNA sequence, particularly for parenteral injection (e.g., intramuscular injection). Plasma-Lyte® is a sterile, non-pyrogenic isotonic solution that may be used for intravenous administration. Each 100 mL volume contains 526 mg of Sodium Chloride, USP (NaCl); 502 mg of Sodium Gluconate (C6H11NaO7); 368 mg of Sodium Acetate Trihydrate, USP (C2H3NaO2·3H2O); 37 mg of Potassium Chloride, USP (KCl); and 30 mg of Magnesium Chloride, USP (MgCl2·6H2O). It contains no antimicrobial agents. The pH is preferably adjusted with sodium hydroxide to about 7.4 (6.5 to 8.0).

The injectable formulations used to deliver the GNE-encoding sequence and the bifunctional shRNA sequence may be sterilized, for example, by filtration through a bacterial-retaining filter, or by incorporating sterilizing agents in the form of sterile solid compositions, which can be dissolved or dispersed in sterile water, Plasma-Lyte® or other sterile injectable medium prior to use.

In order to prolong the expression of the GNE-encoding sequence and the bifunctional shRNA sequence within a system (or to prolong the effect thereof), it may be desirable to slow the absorption of the composition from subcutaneous or intramuscular injection. This may be accomplished by the use of a liquid suspension of crystalline or amorphous material with poor water solubility. The rate of absorption of the composition may then depend upon its rate of dissolution, which, in turn, may depend upon crystal size and crystalline form.

Alternatively, delayed absorption of a parenterally administered GNE-encoding sequence and bifunctional shRNA sequence may be accomplished by dissolving or suspending the composition in an oil vehicle. Injectable depot forms may be prepared by forming microencapsule matrices of the GNE-encoding sequence and the bifunctional shRNA sequence in biodegradable polymers such as polylactide-polyglycolide. Depending upon the ratio of the GNE-encoding sequence and the bifunctional shRNA sequence material to polymer and the nature of the particular polymer employed, the rate of the GNE-encoding sequence and the bifunctional shRNA sequence release can be controlled. Examples of other biodegradable polymers include poly(orthoesters) and poly(anhydrides). As described above, depot injectable formulations may also be prepared by entrapping the GNE-encoding sequence and the bifunctional shRNA sequence in liposomes (or even microemulsions) that are compatible with the target body tissues, such as muscular tissue.

According to additional related embodiments of the present invention, methods for treating, preventing, and/or ameliorating the effects of Hereditary Inclusion Body Myopathy (HIBM) are provided. Such methods generally comprise providing a patient with therapeutically effective amounts of a wild-type GNE-encoding nucleic acid sequence and a bifunctional shRNA sequence that knocks down the expression of a mutant GNE in the patient by administration at a location with hereditary inclusion body myopathy in the patient. In certain embodiments, the wild-type GNE-encoding nucleic acid sequence and the bifunctional shRNA sequence may, preferably, be delivered to a patient in connection with a lipid nanoparticle and a carrier similar to that of Plasma-Lyte®, via parenteral injection.

The specific therapeutically effective dose level for any particular patient may depend upon a variety of factors, including the severity of a patient's HIBM disorder; the activity of the specific GNE-encoding sequence and the bifunctional shRNA sequence employed; the delivery vehicle employed; the age, body weight, general health, gender and diet of the patient; the time of administration, route of administration, the rate/speed of administration and rate of excretion of the specific GNE-encoding sequence and the bifunctional shRNA sequence employed; the duration of the treatment; drugs used in combination or contemporaneously with the specific GNE-encoding sequence and the bifunctional shRNA sequence employed; and like factors well-known in the medical arts.

Upon improvement of a patient's condition, a maintenance dose of a GNE-encoding sequence and a bifunctional shRNA sequence may be administered, if necessary. Subsequently, the dosage or frequency of administration, or both, may be reduced, as a function of the symptoms, to a level at which the improved condition is retained when the symptoms have been alleviated to the desired level.

According to yet further embodiments of the invention, novel compositions are provided for expressing the wild-type GNE (e.g., hGNE1 or hGNE2) and the bifunctional shRNA in a system. The compositions preferably include a wild-type GNE-encoding nucleic acid sequence and a bifunctional shRNA sequence. As described herein, the GNE-encoding nucleic acid sequence and/or the bifunctional shRNA sequence may comprise various transcriptional control elements, such as a promoter, termination sequence, and others. Also as described relative to other embodiments of the present invention, the GNE-encoding nucleic acid sequence and/or the bifunctional shRNA sequence may be disposed within or connected to an appropriate vehicle for delivery to a system, such as a liposome or lipid nanoparticle. Still further, according to such embodiments, the delivery vehicle may, optionally, be decorated with agents that are capable of recognizing and binding to target cells or tissues, such as muscle cells or muscle tissues.

### Expression Vector

In some embodiments, the bifunctional shRNA (bi-shRNA) that knocks down the mutant GNE comprises a first stem-loop structure that comprises an siRNA component and a second stem-loop structure that comprises a miRNA component. In some embodiments, the bifunctional shRNA has two mechanistic pathways of action, that of the siRNA and that of the miRNA. Thus, in some embodiments, the bifunctional shRNA described herein is different from a traditional shRNA, i.e., a DNA transcription derived RNA acting by the siRNA mechanism of action or from a “doublet or more shRNA” that refers to two or more shRNAs, each acting against the expression of two or more different genes but in the traditional siRNA mode. In some embodiments, the bi-shRNA incorporates siRNA (cleavage dependent) and miRNA (cleavage-independent) motifs. In some embodiments, the expression vector comprises both the bifunctional shRNA sequence and the wild-type GNE sequence. In some embodiments, the expression vector only comprises the bifunctional shRNA sequence. In some embodiments, the expression vector only comprises the wild-type GNE sequence.

Bifunctional shRNA

An expression vector can comprise a bifunctional shRNA sequence specific for knockdown of a mutant GNE. In some embodiments, the bifunctional shRNA sequence encodes a nucleic acid sequence capable of hybridizing to one or more regions of an mRNA transcript encoding the mutant GNE to inhibit the expression of the mutant GNE via RNA interference.

SEQ ID NO:1 in the table below provides the mRNA transcript encoding wild-type hGNE1. SEQ ID NO:2 in the table below provides the protein sequence of wild-type hGNE1. In some embodiments, a mutant hGNE1 can comprise at least one mutation selected from D176V, V572L, and M712T, relative to the sequence of SEQ ID NO:2. In some embodiments, the mutant hGNE1 comprises the mutation D176V. In some embodiments, the mutant hGNE1 comprises the mutation V572L. In some embodiments, the mutant hGNE1 comprises the mutation M712T.

In some embodiments, a bifunctional shRNA sequence can knockdown the expression of a mutant hGNE1 that comprises all three mutations of D176V, V572L, and M712T. In particular, a bifunctional shRNA sequence that knocks down a mutant hGNE1 with all three mutations can comprise a sequence having at least 90% (e.g., 90%, 91%, 92%, 93%, 94%, 95%, 96%, 97%, 98%, 99%, or 100%) identity to the sequence of SEQ ID NO:3.

In some embodiments, a bifunctional shRNA sequence can knockdown the expression of a mutant hGNE1 that comprises the mutation D176V. In particular, a bifunctional shRNA sequence that knocks down a mutant hGNE1 with mutation D176V can comprise a sequence having at least 90% (e.g., 90%, 91%, 92%, 93%, 94%, 95%, 96%, 97%, 98%, 99%, or 100%) identity to the sequence of SEQ ID NO:4.

In some embodiments, a bifunctional shRNA sequence can knockdown the expression of a mutant hGNE1 that comprises the mutation V572L. In particular, a bifunctional shRNA sequence that knocks down a mutant hGNE1 with mutation V572L can comprise a sequence having at least 90% (e.g., 90%, 91%, 92%, 93%, 94%, 95%, 96%, 97%, 98%, 99%, or 100%) identity to the sequence of SEQ ID NO:5.

In some embodiments, a bifunctional shRNA sequence can knockdown the expression of a mutant hGNE1 that comprises the mutation M712T. In particular, a bifunctional shRNA sequence that knocks down a mutant hGNE1 with mutation M712T can comprise a sequence having at least 90% (e.g., 90%, 91%, 92%, 93%, 94%, 95%, 96%, 97%, 98%, 99%, or 100%) identity to the sequence of SEQ ID NO:6.

In certain embodiments, an expression vector can comprise a wild-type hGNE1 sequence and a bifunctional shRNA sequence that knocks down a mutant hGNE1 having all three mutations of D176V, V572L, and M712T. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO:7.

In certain embodiments, an expression vector can comprise a wild-type hGNE1 sequence and a bifunctional shRNA sequence that knocks down a mutant hGNE1 having all three mutations of D176V, V572L, and M712T. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO:12.

In certain embodiments, an expression vector can comprise a wild-type hGNE1 sequence and a bifunctional shRNA sequence that knocks down a mutant hGNE1 having the mutation D176V. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO:8.

In certain embodiments, an expression vector can comprise a wild-type hGNE1 sequence and a bifunctional shRNA sequence that knocks down a mutant hGNE1 having the mutation D176V. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO: 13.

In certain embodiments, an expression vector can comprise a wild-type hGNE1 sequence and a bifunctional shRNA sequence that knocks down a mutant hGNE1 having the mutation V572L. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO:9.

In certain embodiments, an expression vector can comprise a wild-type hGNE1 sequence and a bifunctional shRNA sequence that knocks down a mutant hGNE1 having the mutation V572L. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO: 14.

In certain embodiments, an expression vector can comprise a wild-type hGNE1 sequence and a bifunctional shRNA sequence that knocks down a mutant hGNE1 having the mutation M712T. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO: 10.

In certain embodiments, an expression vector can comprise a wild-type hGNE1 sequence and a bifunctional shRNA sequence that knocks down a mutant hGNE1 having the mutation M712T. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO: 15.

In certain embodiments, an expression vector can comprise a wild-type hGNE2 sequence and a bifunctional shRNA sequence that knocks down a mutant hGNE2 having all three mutations of D207V, V603L, and M743T.

In certain embodiments, an expression vector can comprise a wild-type hGNE2 sequence and a bifunctional shRNA sequence that knocks down a mutant hGNE2 having the mutation D207V.

In certain embodiments, an expression vector can comprise a wild-type hGNE2 sequence and a bifunctional shRNA sequence that knocks down a mutant hGNE2 having the mutation V603L.

In certain embodiments, an expression vector can comprise a wild-type hGNE2 sequence and a bifunctional shRNA sequence that knocks down a mutant hGNE2 having the mutation M743T. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO:20. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO:21. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO:22. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO:23. An example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO:25. An example of such an expression vector can be in a Doggybone backbone and can comprises a sequence having at least 90% (e.g., 90%, 91%, 92%, 93%, 94%, 95%, 96%, 97%, 98%, 99%, or 100%) identity to the sequence of SEQ ID NO:26.

In certain embodiments, an expression vector can comprise a wild-type GNE (e.g., hGNE1 or hGNE2) sequence. In some embodiments, an example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO:11. In some embodiments, an example of such an expression vector can be in a pUMVC3 backbone and can comprises a sequence having at least 90% (e.g., 90%, 91%, 92%, 93%, 94%, 95%, 96%, 97%, 98%, 99%, or 100%) identity to the sequence of SEQ ID NO:29.

In some embodiments, the expression vector further comprises a promoter, e.g., the promoter is a cytomegalovirus (CMV) mammalian promoter. In some embodiments, the mammalian CMV promoter comprises a CMV immediate early (IE) 5′ UTR enhancer sequence and a CMV IE Intron A. In further embodiments, the expression vector further comprises a CMV enhancer sequence and a CMV intron sequence.

The first insert and the second insert in the expression vector can be operably linked to the promoter. In particular embodiments, the expression vector further comprises a nucleic acid sequence encoding a picornaviral 2A ribosomal skip peptide between the first and the second nucleic acid inserts.

In some embodiments, the bi-shRNA is capable of hybridizing to one of more regions of an mRNA transcript encoding the mutant GNE (e.g., mutant hGNE1, or mutant hGNE2). In some embodiments, the mRNA transcript encoding the mutant hGNE1 is a nucleic acid sequence of SEQ ID NO:16. In some embodiments, the mRNA transcript encoding the mutant hGNE1 is a nucleic acid sequence of SEQ ID NO:17. In some embodiments, the mRNA transcript encoding the mutant hGNE1 is a nucleic acid sequence of SEQ ID NO:18. In some embodiments, the mRNA transcript encoding the mutant hGNE1 is a nucleic acid sequence of SEQ ID NO:19. In some embodiments, the one or more regions of the mRNA transcript encoding the mutant hGNE1 that are targeted by the bifunctional shRNA are selected from nucleotides 526-528, 1714-1716, and 2134-2136 of SEQ ID NO:16. In some embodiments, the expression vector targets the coding region of the mutant GNE mRNA transcript, the 3′ UTR region sequence of the mutant GNE mRNA transcript, or both the coding sequence and the 3′ UTR sequence of the mutant GNE mRNA transcript simultaneously. In some embodiments, the bi-shRNA comprises a sequence having at least 90% (e.g., 90%, 91%, 92%, 93%, 94%, 95%, 96%, 97%, 98%, 99%, or 100%) identity to any one of SEQ ID NOS:3-6. In some embodiments, the bi-shRNA comprises a sequence having at least 90% (e.g., 90%, 91%, 92%, 93%, 94%, 95%, 96%, 97%, 98%, 99%, or 100%) identity to any one of SEQ ID NOS:31 and 32. In some embodiments, a bi-shRNA capable of hybridizing to one or more regions of an mRNA transcript encoding the mutant GNE is referred to herein as bi-shRNA^{mutGNE}. In some embodiments, the bi-shRNA^{mutGNE} comprises or consists of two stem-loop structures each with a miR-30a loop. In some embodiments, a first stem-loop structure of the two stem-loop structures comprises complementary guiding strand and passenger strand. In some embodiments, the second stem-loop structure of the two stem-loop structures comprises three mismatches in the passenger strand. In some embodiments, the three mismatches are at positions 9 to 11 in the passenger strand. In some embodiments, the bi-shRNA^{mutGNE} comprises or consists of two stem-loop structures each with a miR-17/92 cluster backbone. In some embodiments, a first stem-loop structure of the two stem-loop structures comprises complementary guiding strand and passenger strand. In some embodiments, the second stem-loop structure of the two stem-loop structures comprises three mismatches in the passenger strand. In some embodiments, the three mismatches are at positions 9 to 11 in the passenger strand.

TABLE 1
Sequences

| SEQ ID | | |
|---|---|---|
| NO: | Description | Sequence |
| 1 | mRNA transcript | ATGGAGAAGAATGGAAATAACCGAAAGCTGCGGGTTTGTGTTGCTACTTGTAACCGTGCAGATTA |
| | encoding wild-type | TTCTAAACTTGCCCCGATCATGTTTGGCATTAAAACCGAACCTGAGTTCTTTGAACTTGATGTTG |
| | hGNE1 | TGGTACTTGGCTCTCACCTGATAGATGACTATGGAAATACATATCGAATGATTGAACAAGATGAC |
| | | TTTGACATTAACACCAGGCTACACACAATTGTGAGGGGAGAAGATGAGGCAGCCATGGTGGAGTC |
| | | AGTAGGCCTGGCCCTAGTGAAGCTGCCAGATGTCCTTAATCGCCTGAAGCCTGATATCATGATTG |
| | | TTCATGGAGACAGGTTTGATGCCCTGGCTCTGGCCACATCTGCTGCCTTGATGAACATCCGAATC |
| | | CTTCACATTGAAGGTGGGGAAGTCAGTGGGACCATTGATGACTCTATCAGACATGCCATAACAAA |
| | | ACTGGCTCATTATCATGTGTGCTGCACCCGCAGTGCAGAGCAGCACCTGATATCCATGTGTGAGG |
| | | ACCATGATCGCATCCTTTTGGCAGGCTGCCCTTCCTATGACAAACTTCTCTCAGCCAAGAACAAA |
| | | GACTACATGAGCATCATTCGCATGTGGCTAGGTGATGATGTAAAATCTAAAGATTACATTGTTGC |
| | | ACTACAGCACCCTGTGACCACTGACATTAAGCATTCCATAAAAATGTTTGAATTAACATTGGATG |
| | | CACTTATCTCATTTAACAAGCGGACCCTAGTCCTGTTTCCAAATATTGACGCAGGGAGCAAAGAG |
| | | ATGGTTCGAGTGATGCGGAAGAAGGGCATTGAGCATCATCCCAACTTTCGTGCAGTTAAACACGT |
| | | CCCATTTGACCAGTTTATACAGTTGGTTGCCCATGCTGGCTGTATGATTGGGAACAGCAGCTGTG |
| | | GGGTTCGAGAAGTTGGAGCTTTTGGAACACCTGTGATCAACCTGGGAACACGTCAGATTGGAAGA |
| | | GAAACAGGGGAGAATGTTCTTCATGTCCGGGATGCTGACACCCAAGACAAAATATTGCAAGCACT |
| | | GCACCTTCAGTTTGGTAAACAGTACCCTTGTTCAAAGATATATGGGGATGGAAATGCTGTTCCAA |
| | | GGATTTTGAAGTTTCTCAAATCTATCGATCTTCAAGAGCCACTGCAAAAGAAATTCTGCTTTCCT |
| | | CCTGTGAAGGAGAATATCTCTCAAGATATTGACCATATTCTTGAAACTCTAAGTGCCTTGGCCGT |
| | | TGATCTTGGCGGGACGAACCTCCGAGTTGCAATAGTCAGCATGAAGGGTGAAATAGTTAAGAAGT |
| | | ATACTCAGTTCAATCCTAAAACCTATGAAGAGAGGATTAATTTAATCCTACAGATGTGTGTGGAA |
| | | GCTGCAGCAGAAGCTGTAAAACTGAACTGCAGAATTTTGGGAGTAGGCATTTCCACAGGTGGCCG |
| | | TGTAAATCCTCGGGAAGGAATTGTGCTGCATTCAACCAAACTGATCCAAGAGTGGAACTCTGTGG |
| | | ACCTTAGGACCCCCCTTTCTGACACTTTGCATCTCCCTGTGTGGGTAGACAATGATGGCAACTGT |
| | | GCTGCCCTGGCGGAAAGGAAATTTGGCCAAGGAAAGGGACTGGAAAACTTTGTTACACTTATCAC |
| | | AGGCACAGGAATCGGTGGTGGAATTATCCATCAGCATGAATTGATCCACGGAAGCTCCTTCTGTG |
| | | CTGCAGAACTGGGCCACCTTGTTGTGTCTCTGGATGGGCCTGATTGTTCCTGTGGAAGCCATGGG |
| | | TGCATTGAAGCATACGCCTCTGGAATGGCCTTGCAGAGGGAGGCAAAAAAGCTCCATGATGAGGA |
| | | CCTGCTCTTGGTGGAAGGGATGTCAGTGCCAAAAGATGAGGCTGTGGGTGCGCTCCATCTCATCC |
| | | AAGCTGCGAAACTTGGCAATGCGAAGGCCCAGAGCATCCTAAGAACAGCTGGAACAGCTTTGGGT |
| | | CTTGGGGTTGTGAACATCCTCCATACCATGAATCCCTCCCTTGTGATCCTCTCCGGAGTCCTGGC |
| | | CAGTCACTATATCCACATTGTCAAAGACGTCATTCGCCAGCAGGCCTTGTCCTCCGTGCAGGACG |
| | | TGGATGTGGTGGTTTCGGATTTGGTTGACCCCGCCCTGCTGGGTGCTGCCAGCATGGTTCTGGAC |
| | | TACACAACACGCAGGATCTACTAG |
| 2 | Protein sequence of | MEKNGNNRKLRVCVATCNRADYSKLAPIMFGIKTEPEFFELDVVVLGSHLIDDYGNTYRMIEQDD |
| | wild-type hGNE1 | FDINTRLHTIVRGEDEAAMVESVGLALVKLPDVLNRLKPDIMIVHGDRFDALALATSAALMNIRI |
| | (D176, V572, and | LHIEGGEVSGTIDDSIRHAITKLAHYHVCCTRSAEQHLISMCEDHDRILLAGCPSYDKLLSAKNK |
| | M712 are in bold) | DYMSIIRMWLGDDVKSKDYIVALQHPVTTDIKHSIKMFELTLDALISFNKRTLVLFPNIDAGSKE |
| | | MVRVMRKKGIEHHPNFRAVKHVPFDQFIQLVAHAGCMIGNSSCGVREVGAFGTPVINLGTRQIGR |
| | | ETGENVLHVRDADTQDKILQALHLQFGKQYPCSKIYGDGNAVPRILKFLKSIDLQEPLQKKFCFP |
| | | PVKENISQDIDHILETLSALAVDLGGTNLRVAIVSMKGEIVKKYTQFNPKTYEERINLILQMCVE |
| | | AAAEAVKLNCRILGVGISTGGRVNPREGIVLHSTKLIQEWNSVDLRTPLSDTLHLPVWVDNDGNC |
| | | AALAERKFGQGKGLENFVTLITGTGIGGGIIHQHELIHGSSFCAAELGHLVVSLDGPDCSCGSHG |
| | | CIEAYASGMALQREAKKLHDEDLLLVEGMSVPKDEAVGALHLIQAAKLGNAKAQSILRTAGTALG |
| | | LGVVNILHTMNPSLVILSGVLASHYIHIVKDVIRQQALSSVQDVDVVVSDLVDPALLGAASMVLD |
| | | YTTRRIY |
| 3 | bi-shRNA Triple | TTTCTTCCCCATTAGGGATTATGCTGAATTTGTATGGTTTATAGTTGTTAGAGTTTGAGGTGTTA |
| | Knockdown: (M712T- | ATTCTAATTATCTATTTCAAATTTAGCAGGAAAAAAGAGAACATCACCTTGTAAAACTGAAGATT |
| | D176V-V572L) | GTGACCAGTCAGAATAATGTCCTGCTGGGTGCTGCCAGCACGGAGATATGTGCATCTCCGTGCTG |
| | | GCAGCACCCAGCAGGCATTATGGTGACAGCTGCCTCGGGAAGCCAAGTTGGGCTTTAAAGTGCAG |
| | | GGCCTGCTGATGTTGAGTGCTTTTTGTTCCATGCTGTGTATTGCCATCATCGAGTGAAGTAGATT |
| | | AGCATCTCCGTGCTGGCAGCACCCAGCAGGCATAAGAAGTTATGTATTCATCCAATAATTCAAGC |
| | | CAAGCAAGTATATAGGTGTTTTAATAGTTTTTGTTTGCAGTCCTCTGTTATCCATGTGTGAGGAC |
| | | CATGTTCAGAAGAATGTACTGAACATGGTCCTCACACATGGATTGGTGGCCTGCTATTTCCTTCA |
| | | AATGAATGATTTTTACTAATTTTGTGTACTTTTATTGTGTCGATGTAGAATCTGCCTGGTCTATC |
| | | TGATGTGACAGCTTCTGTAGCACTATATCTGTGTACAGACTATGATCTGTTTAGTTATCAGAACA |
| | | TGGTCCTCACACATGGATTACTGCTAGCTGTAGAACTCCAGCTTCGGCCTGTCGCCCAATCAAAC |
| | | TGTCCTGTTACTGAACACTGTTCTATGGTTCAGAACTGGGCCACCTTGTTCTGGCTGTGATATTC |
| | | TGCCAGAACAAGGTGGCCCAGTTCTGCTGTGGTAGTGAAAAGTCTGTAGAAAAGTAAGGGAAACT |
| | | CAAACCCCTTTCTACACATCACTGGTTAACATTGAATTGCCGTTTCTGTATGGCAGAACAAGGTG |
| | | GCCCAGTTCTGGAGTTTGGTGGGGATTGTGACCAGAAGATTTTGAAAATTAAATATTACTGAAGA |
| | | TTTCGACTTCCACTGTTAAATGTACAAGATACATGAAATATTAAAGAAAATGTGTAACTTTTTGT |
| | | GTAAATACATCTTGT |
| 4 | bi-shRNA Single | TTTCTTCCCCATTAGGGATTATGCTGAATTTGTATGGTTTATAGTTGTTAGAGTTTGAGGTGTTA |
| | Knockdown: (D176V) | ATTCTAATTATCTATTTCAAATTTAGCAGGAAAAAAGAGAACATCACCTTGTAAAACTGAAGATT |
| | | GTGACCAGCAGTCCTCTGTTATCCATGTGTGAGGACCATGTTCAGAAGAATGTACTGAACATGGT |
| | | CCTCACACATGGATTGGTGGCCTGCTATTTCCTTCAAATGAATGATTTTTACTAATTTTGTGTAC |
| | | TTTTATTGTGTCGATGTAGAATCTGCCTGGTCTATCTGATGTGACAGCTTCTGTAGCACTATATC |
| | | TGTGTACAGACTATGATCTGTTTAGTTATCAGAACATGGTCCTCACACATGGATTACTGCTAGCT |
| | | GTAGAACTCCAGCTTCGGCCTGTCGCCCAATCAAACTGTCCTGTTACTGAATGGGGATTGTGACC |
| | | AGAAGATTTTGAAAATTAAATATTACTGAAGATTTCGACTTCCACTGTTAAATGTACAAGATACA |
| | | TGAAATATTAAAGAAAATGTGTAACTTTTTGTGTAAATACATCTTGT |
| 5 | bi-shRNA Single | TTTCTTCCCCATTAGGGATTATGCTGAATTTGTATGGTTTATAGTTGTTAGAGTTTGAGGTGTTA |
| | Knockdown: (V572L) | ATTCTAATTATCTATTTCAAATTTAGCAGGAAAAAAGAGAACATCACCTTGTAAAACTGAAGATT |
| | | GTGACCATAGCTGTAGAACTCCAGCTTCGGCCTGTCGCCCAATCAAACTGTCCTGTTACTGAACA |
| | | CTGTTCTATGGTTCAGAACTGGGCCACCTTGTTCTGGCTGTGATATTCTGCCAGAACAAGGTGGC |
| | | CCAGTTCTGCTGTGGTAGTGAAAAGTCTGTAGAAAAGTAAGGGAAACTCAAACCCCTTTCTACAC |
| | | ATCACTGGTTAACATTGAATTGCCGTTTCTGTATGGCAGAACAAGGTGGCCCAGTTCTGGAGTTT |
| | | GGTGGGGATTGTGACCAGAAGATTTTGAAAATTAAATATTACTGAAGATTTCGACTTCCACTGTT |
| | | AAATGTACAAGATACATGAAATATTAAAGAAAATGTGTAACTTTTTGTGTAAATACATCTTGT |
| 6 | bi-shRNA Single | TTTCTTCCCCATTAGGGATTATGCTGAATTTGTATGGTTTATAGTTGTTAGAGTTTGAGGTGTTA |
| | Knockdown: (M712T) | ATTCTAATTATCTATTTCAAATTTAGCAGGAAAAAAGAGAACATCACCTTGTAAAACTGAAGATT |
| | | GTGACCAGTCAGAATAATGTCCTGCTGGGTGCTGCCAGCACGGAGATATGTGCATCTCCGTGCTG |
| | | GCAGCACCCAGCAGGCATTATGGTGACAGCTGCCTCGGGAAGCCAAGTTGGGCTTTAAAGTGCAG |
| | | GGCCTGCTGATGTTGAGTGCTTTTTGTTCCATGCTGTGTATTGCCATCATCGAGTGAAGTAGATT |
| | | AGCATCTCCGTGCTGGCAGCACCCAGCAGGCATAAGAAGTTATGTATTCATCCAATAATTCAAGC |
| | | CAAGCAAGTATATAGGTGTTTTAATAGTTTTTGTTTTGGGGATTGTGACCAGAAGATTTTGAAAA |
| | | TTAAATATTACTGAAGATTTCGACTTCCACTGTTAAATGTACAAGATACATGAAATATTAAAGAA |
| | | AATGTGTAACTTTTTGTGTAAATACATCTTGT |
| 7 | Vector expressing | tggccattgcatacgttgtatccatatcataatatgtacatttatattggctcatgtccaacatt |
| | hGNE1 and | accgccatgttgacattgattattgactagttattaatagtaatcaattacggggtcattagttc |
| | knockdown three | atagcccatatatggagttccgcgttacataacttacggtaaatggcccgcctggctgaccgccc |
| | hGNE1 mutations in | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | | atatgccaagtacgccccctattgacgtcaatgacggtaaatggcccgcctggcattatgcccag |
| | | tacatgaccttatgggactttcctacttggcagtacatctacgtattagtcatcgctattaccat |
| | | ggtgatgcggttttggcagtacatcaatgggcgtggatagcggtttgactcacggggatttccaa |
| | | gtctccaccccattgacgtcaatgggagtttgttttggcaccaaaatcaacgggactttccaaaa |
| | | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacGTAAGTACCGCCTATAGACTCTATAGGCACACCCCTTTGGCTCTTATGCA |
| | | TGCTATACTGTTTTTGGCTTGGGGCCTATACACCCCCGCTTCCTTATGCTATAGGTGATGGTATA |
| | | GCTTAGCCTATAGGTGTGGGTTATTGACCATTATTGACCACTCCTTTCTTCCCCATTAGGGATTA |
| | | TGCTGAATTTGTATGGTTTATAGTTGTTAGAGTTTGAGGTGTTAATTCTAATTATCTATTTCAAA |
| | | TTTAGCAGGAAAAAAGAGAACATCACCTTGTAAAACTGAAGATTGTGACCAGTCAGAATAATGTC |
| | | CTGCTGGGTGCTGCCAGCACGGAGATATGTGCATCTCCGTGCTGGCAGCACCCAGCAGGCATTAT |
| | | GGTGACAGCTGCCTCGGGAAGCCAAGTTGGGCTTTAAAGTGCAGGGCCTGCTGATGTTGAGTGCT |
| | | TTTTGTTCCATGCTGTGTATTGCCATCATCGAGTGAAGTAGATTAGCATCTCCGTGCTGGCAGCA |
| | | CCCAGCAGGCATAAGAAGTTATGTATTCATCCAATAATTCAAGCCAAGCAAGTATATAGGTGTTT |
| | | TAATAGTTTTTGTTTGCAGTCCTCTGTTATCCATGTGTGAGGACCATGTTCAGAAGAATGTACTG |
| | | AACATGGTCCTCACACATGGATTGGTGGCCTGCTATTTCCTTCAAATGAATGATTTTTACTAATT |
| | | TTGTGTACTTTTATTGTGTCGATGTAGAATCTGCCTGGTCTATCTGATGTGACAGCTTCTGTAGC |
| | | ACTATATCTGTGTACAGACTATGATCTGTTTAGTTATCAGAACATGGTCCTCACACATGGATTAC |
| | | TGCTAGCTGTAGAACTCCAGCTTCGGCCTGTCGCCCAATCAAACTGTCCTGTTACTGAACACTGT |
| | | TCTATGGTTCAGAACTGGGCCACCTTGTTCTGGCTGTGATATTCTGCCAGAACAAGGTGGCCCAG |
| | | TTCTGCTGTGGTAGTGAAAAGTCTGTAGAAAAGTAAGGGAAACTCAAACCCCTTTCTACACATCA |
| | | CTGGTTAACATTGAATTGCCGTTTCTGTATGGCAGAACAAGGTGGCCCAGTTCTGGAGTTTGGTG |
| | | GGGATTGTGACCAGAAGATTTTGAAAATTAAATATTACTGAAGATTTCGACTTCCACTGTTAAAT |
| | | GTACAAGATACATGAAATATTAAAGAAAATGTGTAACTTTTTGTGTAAATACATCTTGTAACGGT |
| | | GGAGGGCAGTGTAGTCTGAGCAGTACTCGTTGCTGCCGCGCGCGCCACCAGACATAATAGCTGAC |
| | | AGACTAACAGACTGTTCCTTTCCATGGGTCTTTTCTGCAGTCACCGTCGTCTACCCGCGCGCCCG |
| | | AGCGCGGAAACCGAGGGGCAGGCTCGCGCTCTGCCTGCTTCGTGGCGCTTGGTTCGTCCCTCGCC |
| | | CGAGGAGCGCGGTGGCGGCGTGGGAGGGAGCCTCTGACGGCGTCTGGAACTCTATTTTAAGAACC |
| | | TCTCAAAACGAAACAAGCAAATCATGGAGAAGAATGGAAATAACCGAAAGCTGCGGGTTTGTGTT |
| | | GCTACTTGTAACCGTGCAGATTATTCTAAACTTGCCCCGATCATGTTTGGCATTAAAACCGAACC |
| | | TGAGTTCTTTGAACTTGATGTTGTGGTACTTGGCTCTCACCTGATAGATGACTATGGAAATACAT |
| | | ATCGAATGATTGAACAAGATGACTTTGACATTAACACCAGGCTACACACAATTGTGAGGGGAGAA |
| | | GATGAGGCAGCCATGGTGGAGTCAGTAGGCCTGGCCCTAGTGAAGCTGCCAGATGTCCTTAATCG |
| | | CCTGAAGCCTGATATCATGATTGTTCATGGAGACAGGTTTGATGCCCTGGCTCTGGCCACATCTG |
| | | CTGCCTTGATGAACATCCGAATCCTTCACATTGAAGGTGGGGAAGTCAGTGGGACCATTGATGAC |
| | | TCTATCAGACATGCCATAACAAAACTGGCTCATTATCATGTGTGCTGCACCCGCAGTGCAGAGCA |
| | | GCACCTGATATCCATGTGTGAGGACCATGATCGCATCCTTTTGGCAGGCTGCCCTTCCTATGACA |
| | | AACTTCTCTCAGCCAAGAACAAAGACTACATGAGCATCATTCGCATGTGGCTAGGTGATGATGTA |
| | | AAATCTAAAGATTACATTGTTGCACTACAGCACCCTGTGACCACTGACATTAAGCATTCCATAAA |
| | | AATGTTTGAATTAACATTGGATGCACTTATCTCATTTAACAAGCGGACCCTAGTCCTGTTTCCAA |
| | | ATATTGACGCAGGGAGCAAAGAGATGGTTCGAGTGATGCGGAAGAAGGGCATTGAGCATCATCCC |
| | | AACTTTCGTGCAGTTAAACACGTCCCATTTGACCAGTTTATACAGTTGGTTGCCCATGCTGGCTG |
| | | TATGATTGGGAACAGCAGCTGTGGGGTTCGAGAAGTTGGAGCTTTTGGAACACCTGTGATCAACC |
| | | TGGGAACACGTCAGATTGGAAGAGAAACAGGGGAGAATGTTCTTCATGTCCGGGATGCTGACACC |
| | | CAAGACAAAATATTGCAAGCACTGCACCTTCAGTTTGGTAAACAGTACCCTTGTTCAAAGATATA |
| | | TGGGGATGGAAATGCTGTTCCAAGGATTTTGAAGTTTCTCAAATCTATCGATCTTCAAGAGCCAC |
| | | TGCAAAAGAAATTCTGCTTTCCTCCTGTGAAGGAGAATATCTCTCAAGATATTGACCATATTCTT |
| | | GAAACTCTAAGTGCCTTGGCCGTTGATCTTGGCGGGACGAACCTCCGAGTTGCAATAGTCAGCAT |
| | | GAAGGGTGAAATAGTTAAGAAGTATACTCAGTTCAATCCTAAAACCTATGAAGAGAGGATTAATT |
| | | TAATCCTACAGATGTGTGTGGAAGCTGCAGCAGAAGCTGTAAAACTGAACTGCAGAATTTTGGGA |
| | | GTAGGCATTTCCACAGGTGGCCGTGTAAATCCTCGGGAAGGAATTGTGCTGCATTCAACCAAACT |
| | | GATCCAAGAGTGGAACTCTGTGGACCTTAGGACCCCCCTTTCTGACACTTTGCATCTCCCTGTGT |
| | | GGGTAGACAATGATGGCAACTGTGCTGCCCTGGCGGAAAGGAAATTTGGCCAAGGAAAGGGACTG |
| | | GAAAACTTTGTTACACTTATCACAGGCACAGGAATCGGTGGTGGAATTATCCATCAGCATGAATT |
| | | GATCCACGGAAGCTCCTTCTGTGCTGCAGAACTGGGCCACCTTGTTGTGTCTCTGGATGGGCCTG |
| | | ATTGTTCCTGTGGAAGCCATGGGTGCATTGAAGCATACGCCTCTGGAATGGCCTTGCAGAGGGAG |
| | | GCAAAAAAGCTCCATGATGAGGACCTGCTCTTGGTGGAAGGGATGTCAGTGCCAAAAGATGAGGC |
| | | TGTGGGTGCGCTCCATCTCATCCAAGCTGCGAAACTTGGCAATGCGAAGGCCCAGAGCATCCTAA |
| | | GAACAGCTGGAACAGCTTTGGGTCTTGGGGTTGTGAACATCCTCCATACCATGAATCCCTCCCTT |
| | | GTGATCCTCTCCGGAGTCCTGGCCAGTCACTATATCCACATTGTCAAAGACGTCATTCGCCAGCA |
| | | GGCCTTGTCCTCCGTGCAGGACGTGGATGTGGTGGTTTCGGATTTGGTTGACCCCGCCCTGCTGG |
| | | GTGCTGCCAGCATGGTTCTGGACTACACAACACGCAGGATCTACTAGACCTCCAGGAACAGACAT |
| | | GGACCTTCTCTCCAGAGCTCCTGAGTGGAATCAAGTTCTTGTCTTTAGGATGACCGTTTCTTAAC |
| | | AATCAAATCTGGTATTGAACTGCAGGTGACTTTGGCAGAGAAATGTTTTCACTTTTGGTCTCCTC |
| | | TTCCAGAGTCACCTTTCCCCACTCCTATTTTTGgttttccctctgccaaaaattatggggacatc |
| | | atgaagccccttgagcatctgacttctggctaataaaggaaatttattttcattgcaatagtgtg |
| | | ttggaattttttgtgtctctcactcggaaggacatatgggagggcaaatcatttaaaacatcaga |
| | | atgagtatttggtttagagtttggcaacatatgcccattcttccgcttcctcgctcactgactcg |
| | | ctgcgctcggtcgttcggctgcggcgagcggtatcagctcactcaaaggcggtaatacggttatc |
| | | cacagaatcaggggataacgcaggaaagaacatgtgagcaaaaggccagcaaaaggccaggaacc |
| | | gtaaaaaggccgcgttgctggcgtttttccataggctccgcccccctgacgagcatcacaaaaat |
| | | cgacgctcaagtcagaggtggcgaaacccgacaggactataaagataccaggcgtttccccctgg |
| | | aagctccctcgtgcgctctcctgttccgaccctgccgcttaccggatacctgtccgcctttctcc |
| | | cttcgggaagcgtggcgctttctcatagctcacgctgtaggtatctcagttcggtgtaggtcgtt |
| | | cgctccaagctgggctgtgtgcacgaaccccccgttcagcccgaccgctgcgccttatccggtaa |
| | | ctatcgtcttgagtccaacccggtaagacacgacttatcgccactggcagcagccactggtaaca |
| | | ggattagcagagcgaggtatgtaggcggtgctacagagttcttgaagtggtggcctaactacggc |
| | | tacactagaagaacagtatttggtatctgcgctctgctgaagccagttaccttcggaaaaagagt |
| | | tggtagctcttgatccggcaaacaaaccaccgctggtagcggtggtttttttgtttgcaagcagc |
| | | agattacgcgcagaaaaaaaggatctcaagaagatcctttgatcttttctacggggtctgacgct |
| | | cagtggaacgaaaactcacgttaagggattttggtcatgagattatcaaaaaggatcttcaccta |
| | | gatccttttaaattaaaaatgaagttttaaatcaatctaaagtatatatgagtaaacttggtctg |
| | | acagttaccaatgcttaatcagtgaggcacctatctcagcgatctgtctatttcgttcatccata |
| | | gttgcctgactcggggggggggggcgctgaggtctgcctcgtgaagaaggtgttgctgactcata |
| | | ccaggcctgaatcgccccatcatccagccagaaagtgagggagccacggttgatgagagctttgt |
| | | tgtaggtggaccagttggtgattttgaacttttgctttgccacggaacggtctgcgttgtcggga |
| | | agatgcgtgatctgatccttcaactcagcaaaagttcgatttattcaacaaagccgccgtcccgt |
| | | caagtcagcgtaatgctctgccagtgttacaaccaattaaccaattctgattagaaaaactcatc |
| | | gagcatcaaatgaaactgcaatttattcatatcaggattatcaataccatatttttgaaaaagcc |
| | | gtttctgtaatgaaggagaaaactcaccgaggcagttccataggatggcaagatcctggtatcgg |
| | | tctgcgattccgactcgtccaacatcaatacaacctattaatttcccctcgtcaaaaataaggtt |
| | | atcaagtgagaaatcaccatgagtgacgactgaatccggtgagaatggcaaaagcttatgcattt |
| | | ctttccagacttgttcaacaggccagccattacgctcgtcatcaaaatcactcgcatcaaccaaa |
| | | ccgttattcattcgtgattgcgcctgagcgagacgaaatacgcgatcgctgttaaaaggacaatt |
| | | acaaacaggaatcgaatgcaaccggcgcaggaacactgccagcgcatcaacaatattttcacctg |
| | | aatcaggatattcttctaatacctggaatgctgttttcccggggatcgcagtggtgagtaaccat |
| | | gcatcatcaggagtacggataaaatgcttgatggtcggaagaggcataaattccgtcagccagtt |
| | | tagtctgaccatctcatctgtaacatcattggcaacgctacctttgccatgtttcagaaacaact |
| | | ctggcgcatcgggcttcccatacaatcgatagattgtcgcacctgattgcccgacattatcgcga |
| | | gcccatttatacccatataaatcagcatccatgttggaatttaatcgcggcctcgagcaagacgt |
| | | ttcccgttgaatatggctcataacaccccttgtattactgtttatgtaagcagacagttttattg |
| | | ttcatgatgatatatttttatcttgtgcaatgtaacatcagagattttgagacacaacgtggctt |
| | | tccccccccccccattattgaagcatttatcagggttattgtctcatgagcggatacatatttga |
| | | atgtatttagaaaaataaacaaataggggttccgcgcacatttccccgaaaagtgccacctgacg |
| | | tctaagaaaccattattatcatgacattaacctataaaaataggcgtatcacgaggccctttcgt |
| | | ctcgcgcgtttcggtgatgacggtgaaaacctctgacacatgcagctcccggagacggtcacagc |
| | | ttgtctgtaagcggatgccgggagcagacaagcccgtcagggcgcgtcagcgggtgttggcgggt |
| | | gtcggggctggcttaactatgcggcatcagagcagattgtactgagagtgcaccatatgcggtgt |
| | | gaaataccgcacagatgcgtaaggagaaaataccgcatcagattggctat |
| 8 | Vector expressing | tggccattgcatacgttgtatccatatcataatatgtacatttatattggctcatgtccaacatt |
| | hGNE1 and | accgccatgttgacattgattattgactagttattaatagtaatcaattacggggtcattagttc |
| | knockdown hGNE1 | atagcccatatatggagttccgcgttacataacttacggtaaatggcccgcctggctgaccgccc |
| | D176V mutations in | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | | atatgccaagtacgccccctattgacgtcaatgacggtaaatggcccgcctggcattatgcccag |
| | | tacatgaccttatgggactttcctacttggcagtacatctacgtattagtcatcgctattaccat |
| | | ggtgatgcggttttggcagtacatcaatgggcgtggatagcggtttgactcacggggatttccaa |
| | | gtctccaccccattgacgtcaatgggagtttgttttggcaccaaaatcaacgggactttccaaaa |
| | | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacGTAAGTACCGCCTATAGACTCTATAGGCACACCCCTTTGGCTCTTATGCA |
| | | TGCTATACTGTTTTTGGCTTGGGGCCTATACACCCCCGCTTCCTTATGCTATAGGTGATGGTATA |
| | | GCTTAGCCTATAGGTGTGGGTTATTGACCATTATTGACCACTCCTTTCTTCCCCATTAGGGATTA |
| | | TGCTGAATTTGTATGGTTTATAGTTGTTAGAGTTTGAGGTGTTAATTCTAATTATCTATTTCAAA |
| | | TTTAGCAGGAAAAAAGAGAACATCACCTTGTAAAACTGAAGATTGTGACCAGCAGTCCTCTGTTA |
| | | TCCATGTGTGAGGACCATGTTCAGAAGAATGTACTGAACATGGTCCTCACACATGGATTGGTGGC |
| | | CTGCTATTTCCTTCAAATGAATGATTTTTACTAATTTTGTGTACTTTTATTGTGTCGATGTAGAA |
| | | TCTGCCTGGTCTATCTGATGTGACAGCTTCTGTAGCACTATATCTGTGTACAGACTATGATCTGT |
| | | TTAGTTATCAGAACATGGTCCTCACACATGGATTACTGCTAGCTGTAGAACTCCAGCTTCGGCCT |
| | | GTCGCCCAATCAAACTGTCCTGTTACTGAATGGGGATTGTGACCAGAAGATTTTGAAAATTAAAT |
| | | ATTACTGAAGATTTCGACTTCCACTGTTAAATGTACAAGATACATGAAATATTAAAGAAAATGTG |
| | | TAACTTTTTGTGTAAATACATCTTGTAACGGTGGAGGGCAGTGTAGTCTGAGCAGTACTCGTTGC |
| | | TGCCGCGCGCGCCACCAGACATAATAGCTGACAGACTAACAGACTGTTCCTTTCCATGGGTCTTT |
| | | TCTGCAGTCACCGTCGTCTACCCGCGCGCCCGAGCGCGGAAACCGAGGGGCAGGCTCGCGCTCTG |
| | | CCTGCTTCGTGGCGCTTGGTTCGTCCCTCGCCCGAGGAGCGCGGTGGCGGCGTGGGAGGGAGCCT |
| | | CTGACGGCGTCTGGAACTCTATTTTAAGAACCTCTCAAAACGAAACAAGCAAATCATGGAGAAGA |
| | | ATGGAAATAACCGAAAGCTGCGGGTTTGTGTTGCTACTTGTAACCGTGCAGATTATTCTAAACTT |
| | | GCCCCGATCATGTTTGGCATTAAAACCGAACCTGAGTTCTTTGAACTTGATGTTGTGGTACTTGG |
| | | CTCTCACCTGATAGATGACTATGGAAATACATATCGAATGATTGAACAAGATGACTTTGACATTA |
| | | ACACCAGGCTACACACAATTGTGAGGGGAGAAGATGAGGCAGCCATGGTGGAGTCAGTAGGCCTG |
| | | GCCCTAGTGAAGCTGCCAGATGTCCTTAATCGCCTGAAGCCTGATATCATGATTGTTCATGGAGA |
| | | CAGGTTTGATGCCCTGGCTCTGGCCACATCTGCTGCCTTGATGAACATCCGAATCCTTCACATTG |
| | | AAGGTGGGGAAGTCAGTGGGACCATTGATGACTCTATCAGACATGCCATAACAAAACTGGCTCAT |
| | | TATCATGTGTGCTGCACCCGCAGTGCAGAGCAGCACCTGATATCCATGTGTGAGGACCATGATCG |
| | | CATCCTTTTGGCAGGCTGCCCTTCCTATGACAAACTTCTCTCAGCCAAGAACAAAGACTACATGA |
| | | GCATCATTCGCATGTGGCTAGGTGATGATGTAAAATCTAAAGATTACATTGTTGCACTACAGCAC |
| | | CCTGTGACCACTGACATTAAGCATTCCATAAAAATGTTTGAATTAACATTGGATGCACTTATCTC |
| | | ATTTAACAAGCGGACCCTAGTCCTGTTTCCAAATATTGACGCAGGGAGCAAAGAGATGGTTCGAG |
| | | TGATGCGGAAGAAGGGCATTGAGCATCATCCCAACTTTCGTGCAGTTAAACACGTCCCATTTGAC |
| | | CAGTTTATACAGTTGGTTGCCCATGCTGGCTGTATGATTGGGAACAGCAGCTGTGGGGTTCGAGA |
| | | AGTTGGAGCTTTTGGAACACCTGTGATCAACCTGGGAACACGTCAGATTGGAAGAGAAACAGGGG |
| | | AGAATGTTCTTCATGTCCGGGATGCTGACACCCAAGACAAAATATTGCAAGCACTGCACCTTCAG |
| | | TTTGGTAAACAGTACCCTTGTTCAAAGATATATGGGGATGGAAATGCTGTTCCAAGGATTTTGAA |
| | | GTTTCTCAAATCTATCGATCTTCAAGAGCCACTGCAAAAGAAATTCTGCTTTCCTCCTGTGAAGG |
| | | AGAATATCTCTCAAGATATTGACCATATTCTTGAAACTCTAAGTGCCTTGGCCGTTGATCTTGGC |
| | | GGGACGAACCTCCGAGTTGCAATAGTCAGCATGAAGGGTGAAATAGTTAAGAAGTATACTCAGTT |
| | | CAATCCTAAAACCTATGAAGAGAGGATTAATTTAATCCTACAGATGTGTGTGGAAGCTGCAGCAG |
| | | AAGCTGTAAAACTGAACTGCAGAATTTTGGGAGTAGGCATTTCCACAGGTGGCCGTGTAAATCCT |
| | | CGGGAAGGAATTGTGCTGCATTCAACCAAACTGATCCAAGAGTGGAACTCTGTGGACCTTAGGAC |
| | | CCCCCTTTCTGACACTTTGCATCTCCCTGTGTGGGTAGACAATGATGGCAACTGTGCTGCCCTGG |
| | | CGGAAAGGAAATTTGGCCAAGGAAAGGGACTGGAAAACTTTGTTACACTTATCACAGGCACAGGA |
| | | ATCGGTGGTGGAATTATCCATCAGCATGAATTGATCCACGGAAGCTCCTTCTGTGCTGCAGAACT |
| | | GGGCCACCTTGTTGTGTCTCTGGATGGGCCTGATTGTTCCTGTGGAAGCCATGGGTGCATTGAAG |
| | | CATACGCCTCTGGAATGGCCTTGCAGAGGGAGGCAAAAAAGCTCCATGATGAGGACCTGCTCTTG |
| | | GTGGAAGGGATGTCAGTGCCAAAAGATGAGGCTGTGGGTGCGCTCCATCTCATCCAAGCTGCGAA |
| | | ACTTGGCAATGCGAAGGCCCAGAGCATCCTAAGAACAGCTGGAACAGCTTTGGGTCTTGGGGTTG |
| | | TGAACATCCTCCATACCATGAATCCCTCCCTTGTGATCCTCTCCGGAGTCCTGGCCAGTCACTAT |
| | | ATCCACATTGTCAAAGACGTCATTCGCCAGCAGGCCTTGTCCTCCGTGCAGGACGTGGATGTGGT |
| | | GGTTTCGGATTTGGTTGACCCCGCCCTGCTGGGTGCTGCCAGCATGGTTCTGGACTACACAACAC |
| | | GCAGGATCTACTAGACCTCCAGGAACAGACATGGACCTTCTCTCCAGAGCTCCTGAGTGGAATCA |
| | | AGTTCTTGTCTTTAGGATGACCGTTTCTTAACAATCAAATCTGGTATTGAACTGCAGGTGACTTT |
| | | GGCAGAGAAATGTTTTCACTTTTGGTCTCCTCTTCCAGAGTCACCTTTCCCCACTCCTATTTTTG |
| | | gttttccctctgccaaaaattatggggacatcatgaagccccttgagcatctgacttctggctaa |
| | | taaaggaaatttattttcattgcaatagtgtgttggaattttttgtgtctctcactcggaaggac |
| | | atatgggagggcaaatcatttaaaacatcagaatgagtatttggtttagagtttggcaacatatg |
| | | cccattcttccgcttcctcgctcactgactcgctgcgctcggtcgttcggctgcggcgagcggta |
| | | tcagctcactcaaaggcggtaatacggttatccacagaatcaggggataacgcaggaaagaacat |
| | | gtgagcaaaaggccagcaaaaggccaggaaccgtaaaaaggccgcgttgctggcgtttttccata |
| | | ggctccgcccccctgacgagcatcacaaaaatcgacgctcaagtcagaggtggcgaaacccgaca |
| | | ggactataaagataccaggcgtttccccctggaagctccctcgtgcgctctcctgttccgaccct |
| | | gccgcttaccggatacctgtccgcctttctcccttcgggaagcgtggcgctttctcatagctcac |
| | | gctgtaggtatctcagttcggtgtaggtcgttcgctccaagctgggctgtgtgcacgaacccccc |
| | | gttcagcccgaccgctgcgccttatccggtaactatcgtcttgagtccaacccggtaagacacga |
| | | cttatcgccactggcagcagccactggtaacaggattagcagagcgaggtatgtaggcggtgcta |
| | | cagagttcttgaagtggtggcctaactacggctacactagaagaacagtatttggtatctgcgct |
| | | ctgctgaagccagttaccttcggaaaaagagttggtagctcttgatccggcaaacaaaccaccgc |
| | | tggtagcggtggtttttttgtttgcaagcagcagattacgcgcagaaaaaaaggatctcaagaag |
| | | atcctttgatcttttctacggggtctgacgctcagtggaacgaaaactcacgttaagggattttg |
| | | gtcatgagattatcaaaaaggatcttcacctagatccttttaaattaaaaatgaagttttaaatc |
| | | aatctaaagtatatatgagtaaacttggtctgacagttaccaatgcttaatcagtgaggcaccta |
| | | tctcagcgatctgtctatttcgttcatccatagttgcctgactcggggggggggggcgctgaggt |
| | | ctgcctcgtgaagaaggtgttgctgactcataccaggcctgaatcgccccatcatccagccagaa |
| | | agtgagggagccacggttgatgagagctttgttgtaggtggaccagttggtgattttgaactttt |
| | | gctttgccacggaacggtctgcgttgtcgggaagatgcgtgatctgatccttcaactcagcaaaa |
| | | gttcgatttattcaacaaagccgccgtcccgtcaagtcagcgtaatgctctgccagtgttacaac |
| | | caattaaccaattctgattagaaaaactcatcgagcatcaaatgaaactgcaatttattcatatc |
| | | aggattatcaataccatatttttgaaaaagccgtttctgtaatgaaggagaaaactcaccgaggc |
| | | agttccataggatggcaagatcctggtatcggtctgcgattccgactcgtccaacatcaatacaa |
| | | cctattaatttcccctcgtcaaaaataaggttatcaagtgagaaatcaccatgagtgacgactga |
| | | atccggtgagaatggcaaaagcttatgcatttctttccagacttgttcaacaggccagccattac |
| | | gctcgtcatcaaaatcactcgcatcaaccaaaccgttattcattcgtgattgcgcctgagcgaga |
| | | cgaaatacgcgatcgctgttaaaaggacaattacaaacaggaatcgaatgcaaccggcgcaggaa |
| | | cactgccagcgcatcaacaatattttcacctgaatcaggatattcttctaatacctggaatgctg |
| | | ttttcccggggatcgcagtggtgagtaaccatgcatcatcaggagtacggataaaatgcttgatg |
| | | gtcggaagaggcataaattccgtcagccagtttagtctgaccatctcatctgtaacatcattggc |
| | | aacgctacctttgccatgtttcagaaacaactctggcgcatcgggcttcccatacaatcgataga |
| | | ttgtcgcacctgattgcccgacattatcgcgagcccatttatacccatataaatcagcatccatg |
| | | ttggaatttaatcgcggcctcgagcaagacgtttcccgttgaatatggctcataacaccccttgt |
| | | attactgtttatgtaagcagacagttttattgttcatgatgatatatttttatcttgtgcaatgt |
| | | aacatcagagattttgagacacaacgtggctttccccccccccccattattgaagcatttatcag |
| | | ggttattgtctcatgagcggatacatatttgaatgtatttagaaaaataaacaaataggggttcc |
| | | gcgcacatttccccgaaaagtgccacctgacgtctaagaaaccattattatcatgacattaacct |
| | | ataaaaataggcgtatcacgaggccctttcgtctcgcgcgtttcggtgatgacggtgaaaacctc |
| | | tgacacatgcagctcccggagacggtcacagcttgtctgtaagcggatgccgggagcagacaagc |
| | | ccgtcagggcgcgtcagcgggtgttggcgggtgtcggggctggcttaactatgcggcatcagagc |
| | | agattgtactgagagtgcaccatatgcggtgtgaaataccgcacagatgcgtaaggagaaaatac |
| | | cgcatcagattggctat |
| 9 | Vector expressing | tggccattgcatacgttgtatccatatcataatatgtacatttatattggctcatgtccaacatt |
| | hGNE1 and | accgccatgttgacattgattattgactagttattaatagtaatcaattacggggtcattagttc |
| | knockdown hGNE1 | atagcccatatatggagttccgcgttacataacttacggtaaatggcccgcctggctgaccgccc |
| | V572L mutations in | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | | atatgccaagtacgccccctattgacgtcaatgacggtaaatggcccgcctggcattatgcccag |
| | | tacatgaccttatgggactttcctacttggcagtacatctacgtattagtcatcgctattaccat |
| | | ggtgatgcggttttggcagtacatcaatgggcgtggatagcggtttgactcacggggatttccaa |
| | | gtctccaccccattgacgtcaatgggagtttgttttggcaccaaaatcaacgggactttccaaaa |
| | | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacGTAAGTACCGCCTATAGACTCTATAGGCACACCCCTTTGGCTCTTATGCA |
| | | TGCTATACTGTTTTTGGCTTGGGGCCTATACACCCCCGCTTCCTTATGCTATAGGTGATGGTATA |
| | | GCTTAGCCTATAGGTGTGGGTTATTGACCATTATTGACCACTCCTTTCTTCCCCATTAGGGATTA |
| | | TGCTGAATTTGTATGGTTTATAGTTGTTAGAGTTTGAGGTGTTAATTCTAATTATCTATTTCAAA |
| | | TTTAGCAGGAAAAAAGAGAACATCACCTTGTAAAACTGAAGATTGTGACCATAGCTGTAGAACTC |
| | | CAGCTTCGGCCTGTCGCCCAATCAAACTGTCCTGTTACTGAACACTGTTCTATGGTTCAGAACTG |
| | | GGCCACCTTGTTCTGGCTGTGATATTCTGCCAGAACAAGGTGGCCCAGTTCTGCTGTGGTAGTGA |
| | | AAAGTCTGTAGAAAAGTAAGGGAAACTCAAACCCCTTTCTACACATCACTGGTTAACATTGAATT |
| | | GCCGTTTCTGTATGGCAGAACAAGGTGGCCCAGTTCTGGAGTTTGGTGGGGATTGTGACCAGAAG |
| | | ATTTTGAAAATTAAATATTACTGAAGATTTCGACTTCCACTGTTAAATGTACAAGATACATGAAA |
| | | TATTAAAGAAAATGTGTAACTTTTTGTGTAAATACATCTTGTAACGGTGGAGGGCAGTGTAGTCT |
| | | GAGCAGTACTCGTTGCTGCCGCGCGCGCCACCAGACATAATAGCTGACAGACTAACAGACTGTTC |
| | | CTTTCCATGGGTCTTTTCTGCAGTCACCGTCGTCTACCCGCGCGCCCGAGCGCGGAAACCGAGGG |
| | | GCAGGCTCGCGCTCTGCCTGCTTCGTGGCGCTTGGTTCGTCCCTCGCCCGAGGAGCGCGGTGGCG |
| | | GCGTGGGAGGGAGCCTCTGACGGCGTCTGGAACTCTATTTTAAGAACCTCTCAAAACGAAACAAG |
| | | CAAATCATGGAGAAGAATGGAAATAACCGAAAGCTGCGGGTTTGTGTTGCTACTTGTAACCGTGC |
| | | AGATTATTCTAAACTTGCCCCGATCATGTTTGGCATTAAAACCGAACCTGAGTTCTTTGAACTTG |
| | | ATGTTGTGGTACTTGGCTCTCACCTGATAGATGACTATGGAAATACATATCGAATGATTGAACAA |
| | | GATGACTTTGACATTAACACCAGGCTACACACAATTGTGAGGGGAGAAGATGAGGCAGCCATGGT |
| | | GGAGTCAGTAGGCCTGGCCCTAGTGAAGCTGCCAGATGTCCTTAATCGCCTGAAGCCTGATATCA |
| | | TGATTGTTCATGGAGACAGGTTTGATGCCCTGGCTCTGGCCACATCTGCTGCCTTGATGAACATC |
| | | CGAATCCTTCACATTGAAGGTGGGGAAGTCAGTGGGACCATTGATGACTCTATCAGACATGCCAT |
| | | AACAAAACTGGCTCATTATCATGTGTGCTGCACCCGCAGTGCAGAGCAGCACCTGATATCCATGT |
| | | GTGAGGACCATGATCGCATCCTTTTGGCAGGCTGCCCTTCCTATGACAAACTTCTCTCAGCCAAG |
| | | AACAAAGACTACATGAGCATCATTCGCATGTGGCTAGGTGATGATGTAAAATCTAAAGATTACAT |
| | | TGTTGCACTACAGCACCCTGTGACCACTGACATTAAGCATTCCATAAAAATGTTTGAATTAACAT |
| | | TGGATGCACTTATCTCATTTAACAAGCGGACCCTAGTCCTGTTTCCAAATATTGACGCAGGGAGC |
| | | AAAGAGATGGTTCGAGTGATGCGGAAGAAGGGCATTGAGCATCATCCCAACTTTCGTGCAGTTAA |
| | | ACACGTCCCATTTGACCAGTTTATACAGTTGGTTGCCCATGCTGGCTGTATGATTGGGAACAGCA |
| | | GCTGTGGGGTTCGAGAAGTTGGAGCTTTTGGAACACCTGTGATCAACCTGGGAACACGTCAGATT |
| | | GGAAGAGAAACAGGGGAGAATGTTCTTCATGTCCGGGATGCTGACACCCAAGACAAAATATTGCA |
| | | AGCACTGCACCTTCAGTTTGGTAAACAGTACCCTTGTTCAAAGATATATGGGGATGGAAATGCTG |
| | | TTCCAAGGATTTTGAAGTTTCTCAAATCTATCGATCTTCAAGAGCCACTGCAAAAGAAATTCTGC |
| | | TTTCCTCCTGTGAAGGAGAATATCTCTCAAGATATTGACCATATTCTTGAAACTCTAAGTGCCTT |
| | | GGCCGTTGATCTTGGCGGGACGAACCTCCGAGTTGCAATAGTCAGCATGAAGGGTGAAATAGTTA |
| | | AGAAGTATACTCAGTTCAATCCTAAAACCTATGAAGAGAGGATTAATTTAATCCTACAGATGTGT |
| | | GTGGAAGCTGCAGCAGAAGCTGTAAAACTGAACTGCAGAATTTTGGGAGTAGGCATTTCCACAGG |
| | | TGGCCGTGTAAATCCTCGGGAAGGAATTGTGCTGCATTCAACCAAACTGATCCAAGAGTGGAACT |
| | | CTGTGGACCTTAGGACCCCCCTTTCTGACACTTTGCATCTCCCTGTGTGGGTAGACAATGATGGC |
| | | AACTGTGCTGCCCTGGCGGAAAGGAAATTTGGCCAAGGAAAGGGACTGGAAAACTTTGTTACACT |
| | | TATCACAGGCACAGGAATCGGTGGTGGAATTATCCATCAGCATGAATTGATCCACGGAAGCTCCT |
| | | TCTGTGCTGCAGAACTGGGCCACCTTGTTGTGTCTCTGGATGGGCCTGATTGTTCCTGTGGAAGC |
| | | CATGGGTGCATTGAAGCATACGCCTCTGGAATGGCCTTGCAGAGGGAGGCAAAAAAGCTCCATGA |
| | | TGAGGACCTGCTCTTGGTGGAAGGGATGTCAGTGCCAAAAGATGAGGCTGTGGGTGCGCTCCATC |
| | | TCATCCAAGCTGCGAAACTTGGCAATGCGAAGGCCCAGAGCATCCTAAGAACAGCTGGAACAGCT |
| | | TTGGGTCTTGGGGTTGTGAACATCCTCCATACCATGAATCCCTCCCTTGTGATCCTCTCCGGAGT |
| | | CCTGGCCAGTCACTATATCCACATTGTCAAAGACGTCATTCGCCAGCAGGCCTTGTCCTCCGTGC |
| | | AGGACGTGGATGTGGTGGTTTCGGATTTGGTTGACCCCGCCCTGCTGGGTGCTGCCAGCATGGTT |
| | | CTGGACTACACAACACGCAGGATCTACTAGACCTCCAGGAACAGACATGGACCTTCTCTCCAGAG |
| | | CTCCTGAGTGGAATCAAGTTCTTGTCTTTAGGATGACCGTTTCTTAACAATCAAATCTGGTATTG |
| | | AACTGCAGGTGACTTTGGCAGAGAAATGTTTTCACTTTTGGTCTCCTCTTCCAGAGTCACCTTTC |
| | | CCCACTCCTATTTTTGgttttccctctgccaaaaattatggggacatcatgaagccccttgagca |
| | | tctgacttctggctaataaaggaaatttattttcattgcaatagtgtgttggaattttttgtgtc |
| | | tctcactcggaaggacatatgggagggcaaatcatttaaaacatcagaatgagtatttggtttag |
| | | agtttggcaacatatgcccattcttccgcttcctcgctcactgactcgctgcgctcggtcgttcg |
| | | gctgcggcgagcggtatcagctcactcaaaggcggtaatacggttatccacagaatcaggggata |
| | | acgcaggaaagaacatgtgagcaaaaggccagcaaaaggccaggaaccgtaaaaaggccgcgttg |
| | | ctggcgtttttccataggctccgcccccctgacgagcatcacaaaaatcgacgctcaagtcagag |
| | | gtggcgaaacccgacaggactataaagataccaggcgtttccccctggaagctccctcgtgcgct |
| | | ctcctgttccgaccctgccgcttaccggatacctgtccgcctttctcccttcgggaagcgtggcg |
| | | ctttctcatagctcacgctgtaggtatctcagttcggtgtaggtcgttcgctccaagctgggctg |
| | | tgtgcacgaaccccccgttcagcccgaccgctgcgccttatccggtaactatcgtcttgagtcca |
| | | acccggtaagacacgacttatcgccactggcagcagccactggtaacaggattagcagagcgagg |
| | | tatgtaggcggtgctacagagttcttgaagtggtggcctaactacggctacactagaagaacagt |
| | | atttggtatctgcgctctgctgaagccagttaccttcggaaaaagagttggtagctcttgatccg |
| | | gcaaacaaaccaccgctggtagcggtggtttttttgtttgcaagcagcagattacgcgcagaaaa |
| | | aaaggatctcaagaagatcctttgatcttttctacggggtctgacgctcagtggaacgaaaactc |
| | | acgttaagggattttggtcatgagattatcaaaaaggatcttcacctagatccttttaaattaaa |
| | | aatgaagttttaaatcaatctaaagtatatatgagtaaacttggtctgacagttaccaatgctta |
| | | atcagtgaggcacctatctcagcgatctgtctatttcgttcatccatagttgcctgactcggggg |
| | | gggggggcgctgaggtctgcctcgtgaagaaggtgttgctgactcataccaggcctgaatcgccc |
| | | catcatccagccagaaagtgagggagccacggttgatgagagctttgttgtaggtggaccagttg |
| | | gtgattttgaacttttgctttgccacggaacggtctgcgttgtcgggaagatgcgtgatctgatc |
| | | cttcaactcagcaaaagttcgatttattcaacaaagccgccgtcccgtcaagtcagcgtaatgct |
| | | ctgccagtgttacaaccaattaaccaattctgattagaaaaactcatcgagcatcaaatgaaact |
| | | gcaatttattcatatcaggattatcaataccatatttttgaaaaagccgtttctgtaatgaagga |
| | | gaaaactcaccgaggcagttccataggatggcaagatcctggtatcggtctgcgattccgactcg |
| | | tccaacatcaatacaacctattaatttcccctcgtcaaaaataaggttatcaagtgagaaatcac |
| | | catgagtgacgactgaatccggtgagaatggcaaaagcttatgcatttctttccagacttgttca |
| | | acaggccagccattacgctcgtcatcaaaatcactcgcatcaaccaaaccgttattcattcgtga |
| | | ttgcgcctgagcgagacgaaatacgcgatcgctgttaaaaggacaattacaaacaggaatcgaat |
| | | gcaaccggcgcaggaacactgccagcgcatcaacaatattttcacctgaatcaggatattcttct |
| | | aatacctggaatgctgttttcccggggatcgcagtggtgagtaaccatgcatcatcaggagtacg |
| | | gataaaatgcttgatggtcggaagaggcataaattccgtcagccagtttagtctgaccatctcat |
| | | ctgtaacatcattggcaacgctacctttgccatgtttcagaaacaactctggcgcatcgggcttc |
| | | ccatacaatcgatagattgtcgcacctgattgcccgacattatcgcgagcccatttatacccata |
| | | taaatcagcatccatgttggaatttaatcgcggcctcgagcaagacgtttcccgttgaatatggc |
| | | tcataacaccccttgtattactgtttatgtaagcagacagttttattgttcatgatgatatattt |
| | | ttatcttgtgcaatgtaacatcagagattttgagacacaacgtggctttccccccccccccatta |
| | | ttgaagcatttatcagggttattgtctcatgagcggatacatatttgaatgtatttagaaaaata |
| | | aacaaataggggttccgcgcacatttccccgaaaagtgccacctgacgtctaagaaaccattatt |
| | | atcatgacattaacctataaaaataggcgtatcacgaggccctttcgtctcgcgcgtttcggtga |
| | | tgacggtgaaaacctctgacacatgcagctcccggagacggtcacagcttgtctgtaagcggatg |
| | | ccgggagcagacaagcccgtcagggcgcgtcagcgggtgttggcgggtgtcggggctggcttaac |
| | | tatgcggcatcagagcagattgtactgagagtgcaccatatgcggtgtgaaataccgcacagatg |
| | | cgtaaggagaaaataccgcatcagattggctat |
| 10 | Vector expressing | tggccattgcatacgttgtatccatatcataatatgtacatttatattggctcatgtccaacatt |
| | hGNE1 and | accgccatgttgacattgattattgactagttattaatagtaatcaattacggggtcattagttc |
| | knockdown M712T | atagcccatatatggagttccgcgttacataacttacggtaaatggcccgcctggctgaccgccc |
| | hGNE1 mutations in | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | | atatgccaagtacgccccctattgacgtcaatgacggtaaatggcccgcctggcattatgcccag |
| | | tacatgaccttatgggactttcctacttggcagtacatctacgtattagtcatcgctattaccat |
| | | ggtgatgcggttttggcagtacatcaatgggcgtggatagcggtttgactcacggggatttccaa |
| | | gtctccaccccattgacgtcaatgggagtttgttttggcaccaaaatcaacgggactttccaaaa |
| | | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacGTAAGTACCGCCTATAGACTCTATAGGCACACCCCTTTGGCTCTTATGCA |
| | | TGCTATACTGTTTTTGGCTTGGGGCCTATACACCCCCGCTTCCTTATGCTATAGGTGATGGTATA |
| | | GCTTAGCCTATAGGTGTGGGTTATTGACCATTATTGACCACTCCTTTCTTCCCCATTAGGGATTA |
| | | TGCTGAATTTGTATGGTTTATAGTTGTTAGAGTTTGAGGTGTTAATTCTAATTATCTATTTCAAA |
| | | TTTAGCAGGAAAAAAGAGAACATCACCTTGTAAAACTGAAGATTGTGACCAGTCAGAATAATGTC |
| | | CTGCTGGGTGCTGCCAGCACGGAGATATGTGCATCTCCGTGCTGGCAGCACCCAGCAGGCATTAT |
| | | GGTGACAGCTGCCTCGGGAAGCCAAGTTGGGCTTTAAAGTGCAGGGCCTGCTGATGTTGAGTGCT |
| | | TTTTGTTCCATGCTGTGTATTGCCATCATCGAGTGAAGTAGATTAGCATCTCCGTGCTGGCAGCA |
| | | CCCAGCAGGCATAAGAAGTTATGTATTCATCCAATAATTCAAGCCAAGCAAGTATATAGGTGTTT |
| | | TAATAGTTTTTGTTTTGGGGATTGTGACCAGAAGATTTTGAAAATTAAATATTACTGAAGATTTC |
| | | GACTTCCACTGTTAAATGTACAAGATACATGAAATATTAAAGAAAATGTGTAACTTTTTGTGTAA |
| | | ATACATCTTGTAACGGTGGAGGGCAGTGTAGTCTGAGCAGTACTCGTTGCTGCCGCGCGCGCCAC |
| | | CAGACATAATAGCTGACAGACTAACAGACTGTTCCTTTCCATGGGTCTTTTCTGCAGTCACCGTC |
| | | GTCTACCCGCGCGCCCGAGCGCGGAAACCGAGGGGCAGGCTCGCGCTCTGCCTGCTTCGTGGCGC |
| | | TTGGTTCGTCCCTCGCCCGAGGAGCGCGGTGGCGGCGTGGGAGGGAGCCTCTGACGGCGTCTGGA |
| | | ACTCTATTTTAAGAACCTCTCAAAACGAAACAAGCAAATCATGGAGAAGAATGGAAATAACCGAA |
| | | AGCTGCGGGTTTGTGTTGCTACTTGTAACCGTGCAGATTATTCTAAACTTGCCCCGATCATGTTT |
| | | GGCATTAAAACCGAACCTGAGTTCTTTGAACTTGATGTTGTGGTACTTGGCTCTCACCTGATAGA |
| | | TGACTATGGAAATACATATCGAATGATTGAACAAGATGACTTTGACATTAACACCAGGCTACACA |
| | | CAATTGTGAGGGGAGAAGATGAGGCAGCCATGGTGGAGTCAGTAGGCCTGGCCCTAGTGAAGCTG |
| | | CCAGATGTCCTTAATCGCCTGAAGCCTGATATCATGATTGTTCATGGAGACAGGTTTGATGCCCT |
| | | GGCTCTGGCCACATCTGCTGCCTTGATGAACATCCGAATCCTTCACATTGAAGGTGGGGAAGTCA |
| | | GTGGGACCATTGATGACTCTATCAGACATGCCATAACAAAACTGGCTCATTATCATGTGTGCTGC |
| | | ACCCGCAGTGCAGAGCAGCACCTGATATCCATGTGTGAGGACCATGATCGCATCCTTTTGGCAGG |
| | | CTGCCCTTCCTATGACAAACTTCTCTCAGCCAAGAACAAAGACTACATGAGCATCATTCGCATGT |
| | | GGCTAGGTGATGATGTAAAATCTAAAGATTACATTGTTGCACTACAGCACCCTGTGACCACTGAC |
| | | ATTAAGCATTCCATAAAAATGTTTGAATTAACATTGGATGCACTTATCTCATTTAACAAGCGGAC |
| | | CCTAGTCCTGTTTCCAAATATTGACGCAGGGAGCAAAGAGATGGTTCGAGTGATGCGGAAGAAGG |
| | | GCATTGAGCATCATCCCAACTTTCGTGCAGTTAAACACGTCCCATTTGACCAGTTTATACAGTTG |
| | | GTTGCCCATGCTGGCTGTATGATTGGGAACAGCAGCTGTGGGGTTCGAGAAGTTGGAGCTTTTGG |
| | | AACACCTGTGATCAACCTGGGAACACGTCAGATTGGAAGAGAAACAGGGGAGAATGTTCTTCATG |
| | | TCCGGGATGCTGACACCCAAGACAAAATATTGCAAGCACTGCACCTTCAGTTTGGTAAACAGTAC |
| | | CCTTGTTCAAAGATATATGGGGATGGAAATGCTGTTCCAAGGATTTTGAAGTTTCTCAAATCTAT |
| | | CGATCTTCAAGAGCCACTGCAAAAGAAATTCTGCTTTCCTCCTGTGAAGGAGAATATCTCTCAAG |
| | | ATATTGACCATATTCTTGAAACTCTAAGTGCCTTGGCCGTTGATCTTGGCGGGACGAACCTCCGA |
| | | GTTGCAATAGTCAGCATGAAGGGTGAAATAGTTAAGAAGTATACTCAGTTCAATCCTAAAACCTA |
| | | TGAAGAGAGGATTAATTTAATCCTACAGATGTGTGTGGAAGCTGCAGCAGAAGCTGTAAAACTGA |
| | | ACTGCAGAATTTTGGGAGTAGGCATTTCCACAGGTGGCCGTGTAAATCCTCGGGAAGGAATTGTG |
| | | CTGCATTCAACCAAACTGATCCAAGAGTGGAACTCTGTGGACCTTAGGACCCCCCTTTCTGACAC |
| | | TTTGCATCTCCCTGTGTGGGTAGACAATGATGGCAACTGTGCTGCCCTGGCGGAAAGGAAATTTG |
| | | GCCAAGGAAAGGGACTGGAAAACTTTGTTACACTTATCACAGGCACAGGAATCGGTGGTGGAATT |
| | | ATCCATCAGCATGAATTGATCCACGGAAGCTCCTTCTGTGCTGCAGAACTGGGCCACCTTGTTGT |
| | | GTCTCTGGATGGGCCTGATTGTTCCTGTGGAAGCCATGGGTGCATTGAAGCATACGCCTCTGGAA |
| | | TGGCCTTGCAGAGGGAGGCAAAAAAGCTCCATGATGAGGACCTGCTCTTGGTGGAAGGGATGTCA |
| | | GTGCCAAAAGATGAGGCTGTGGGTGCGCTCCATCTCATCCAAGCTGCGAAACTTGGCAATGCGAA |
| | | GGCCCAGAGCATCCTAAGAACAGCTGGAACAGCTTTGGGTCTTGGGGTTGTGAACATCCTCCATA |
| | | CCATGAATCCCTCCCTTGTGATCCTCTCCGGAGTCCTGGCCAGTCACTATATCCACATTGTCAAA |
| | | GACGTCATTCGCCAGCAGGCCTTGTCCTCCGTGCAGGACGTGGATGTGGTGGTTTCGGATTTGGT |
| | | TGACCCCGCCCTGCTGGGTGCTGCCAGCATGGTTCTGGACTACACAACACGCAGGATCTACTAGA |
| | | CCTCCAGGAACAGACATGGACCTTCTCTCCAGAGCTCCTGAGTGGAATCAAGTTCTTGTCTTTAG |
| | | GATGACCGTTTCTTAACAATCAAATCTGGTATTGAACTGCAGGTGACTTTGGCAGAGAAATGTTT |
| | | TCACTTTTGGTCTCCTCTTCCAGAGTCACCTTTCCCCACTCCTATTTTTGgttttccctctgcca |
| | | aaaattatggggacatcatgaagccccttgagcatctgacttctggctaataaaggaaatttatt |
| | | ttcattgcaatagtgtgttggaattttttgtgtctctcactcggaaggacatatgggagggcaaa |
| | | tcatttaaaacatcagaatgagtatttggtttagagtttggcaacatatgcccattcttccgctt |
| | | cctcgctcactgactcgctgcgctcggtcgttcggctgcggcgagcggtatcagctcactcaaag |
| | | gcggtaatacggttatccacagaatcaggggataacgcaggaaagaacatgtgagcaaaaggcca |
| | | gcaaaaggccaggaaccgtaaaaaggccgcgttgctggcgtttttccataggctccgcccccctg |
| | | acgagcatcacaaaaatcgacgctcaagtcagaggtggcgaaacccgacaggactataaagatac |
| | | caggcgtttccccctggaagctccctcgtgcgctctcctgttccgaccctgccgcttaccggata |
| | | cctgtccgcctttctcccttcgggaagcgtggcgctttctcatagctcacgctgtaggtatctca |
| | | gttcggtgtaggtcgttcgctccaagctgggctgtgtgcacgaaccccccgttcagcccgaccgc |
| | | tgcgccttatccggtaactatcgtcttgagtccaacccggtaagacacgacttatcgccactggc |
| | | agcagccactggtaacaggattagcagagcgaggtatgtaggcggtgctacagagttcttgaagt |
| | | ggtggcctaactacggctacactagaagaacagtatttggtatctgcgctctgctgaagccagtt |
| | | accttcggaaaaagagttggtagctcttgatccggcaaacaaaccaccgctggtagcggtggttt |
| | | ttttgtttgcaagcagcagattacgcgcagaaaaaaaggatctcaagaagatcctttgatctttt |
| | | ctacggggtctgacgctcagtggaacgaaaactcacgttaagggattttggtcatgagattatca |
| | | aaaaggatcttcacctagatccttttaaattaaaaatgaagttttaaatcaatctaaagtatata |
| | | tgagtaaacttggtctgacagttaccaatgcttaatcagtgaggcacctatctcagcgatctgtc |
| | | tatttcgttcatccatagttgcctgactcggggggggggggcgctgaggtctgcctcgtgaagaa |
| | | ggtgttgctgactcataccaggcctgaatcgccccatcatccagccagaaagtgagggagccacg |
| | | gttgatgagagctttgttgtaggtggaccagttggtgattttgaacttttgctttgccacggaac |
| | | ggtctgcgttgtcgggaagatgcgtgatctgatccttcaactcagcaaaagttcgatttattcaa |
| | | caaagccgccgtcccgtcaagtcagcgtaatgctctgccagtgttacaaccaattaaccaattct |
| | | gattagaaaaactcatcgagcatcaaatgaaactgcaatttattcatatcaggattatcaatacc |
| | | atatttttgaaaaagccgtttctgtaatgaaggagaaaactcaccgaggcagttccataggatgg |
| | | caagatcctggtatcggtctgcgattccgactcgtccaacatcaatacaacctattaatttcccc |
| | | tcgtcaaaaataaggttatcaagtgagaaatcaccatgagtgacgactgaatccggtgagaatgg |
| | | caaaagcttatgcatttctttccagacttgttcaacaggccagccattacgctcgtcatcaaaat |
| | | cactcgcatcaaccaaaccgttattcattcgtgattgcgcctgagcgagacgaaatacgcgatcg |
| | | ctgttaaaaggacaattacaaacaggaatcgaatgcaaccggcgcaggaacactgccagcgcatc |
| | | aacaatattttcacctgaatcaggatattcttctaatacctggaatgctgttttcccggggatcg |
| | | cagtggtgagtaaccatgcatcatcaggagtacggataaaatgcttgatggtcggaagaggcata |
| | | aattccgtcagccagtttagtctgaccatctcatctgtaacatcattggcaacgctacctttgcc |
| | | atgtttcagaaacaactctggcgcatcgggcttcccatacaatcgatagattgtcgcacctgatt |
| | | gcccgacattatcgcgagcccatttatacccatataaatcagcatccatgttggaatttaatcgc |
| | | ggcctcgagcaagacgtttcccgttgaatatggctcataacaccccttgtattactgtttatgta |
| | | agcagacagttttattgttcatgatgatatatttttatcttgtgcaatgtaacatcagagatttt |
| | | gagacacaacgtggctttccccccccccccattattgaagcatttatcagggttattgtctcatg |
| | | agcggatacatatttgaatgtatttagaaaaataaacaaataggggttccgcgcacatttccccg |
| | | aaaagtgccacctgacgtctaagaaaccattattatcatgacattaacctataaaaataggcgta |
| | | tcacgaggccctttcgtctcgcgcgtttcggtgatgacggtgaaaacctctgacacatgcagctc |
| | | ccggagacggtcacagcttgtctgtaagcggatgccgggagcagacaagcccgtcagggcgcgtc |
| | | agcgggtgttggcgggtgtcggggctggcttaactatgcggcatcagagcagattgtactgagag |
| | | tgcaccatatgcggtgtgaaataccgcacagatgcgtaaggagaaaataccgcatcagattggct |
| | | at |
| 11 | Vector expressing | tggccattgcatacgttgtatccatatcataatatgtacatttatattggctcatgtccaacatt |
| | hGNE1 in pUMVC3 | accgccatgttgacattgattattgactagttattaatagtaatcaattacggggtcattagttc |
| | backbone | atagcccatatatggagttccgcgttacataacttacggtaaatggcccgcctggctgaccgccc |
| | | aacgacccccgcccattgacgtcaataatgacgtatgttcccatagtaacgccaatagggacttt |
| | | ccattgacgtcaatgggtggagtatttacggtaaactgcccacttggcagtacatcaagtgtatc |
| | | atatgccaagtacgccccctattgacgtcaatgacggtaaatggcccgcctggcattatgcccag |
| | | tacatgaccttatgggactttcctacttggcagtacatctacgtattagtcatcgctattaccat |
| | | ggtgatgcggttttggcagtacatcaatgggcgtggatagcggtttgactcacggggatttccaa |
| | | gtctccaccccattgacgtcaatgggagtttgttttggcaccaaaatcaacgggactttccaaaa |
| | | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacGTAAGTACCGCCTATAGACTCTATAGGCACACCCCTTTGGCTCTTATGCA |
| | | TGCTATACTGTTTTTGGCTTGGGGCCTATACACCCCCGCTTCCTTATGCTATAGGTGATGGTATA |
| | | GCTTAGCCTATAGGTGTGGGTTATTGACCATTATTGACCACTCCAACGGTGGAGGGCAGTGTAGT |
| | | CTGAGCAGTACTCGTTGCTGCCGCGCGCGCCACCAGACATAATAGCTGACAGACTAACAGACTGT |
| | | TCCTTTCCATGGGTCTTTTCTGCAGTCACCGTCGTCTACCCGCGCGCCCGAGCGCGGAAACCGAG |
| | | GGGCAGGCTCGCGCTCTGCCTGCTTCGTGGCGCTTGGTTCGTCCCTCGCCCGAGGAGCGCGGTGG |
| | | CGGCGTGGGAGGGAGCCTCTGACGGCGTCTGGAACTCTATTTTAAGAACCTCTCAAAACGAAACA |
| | | AGCAAATCATGGAGAAGAATGGAAATAACCGAAAGCTGCGGGTTTGTGTTGCTACTTGTAACCGT |
| | | GCAGATTATTCTAAACTTGCCCCGATCATGTTTGGCATTAAAACCGAACCTGAGTTCTTTGAACT |
| | | TGATGTTGTGGTACTTGGCTCTCACCTGATAGATGACTATGGAAATACATATCGAATGATTGAAC |
| | | AAGATGACTTTGACATTAACACCAGGCTACACACAATTGTGAGGGGAGAAGATGAGGCAGCCATG |
| | | GTGGAGTCAGTAGGCCTGGCCCTAGTGAAGCTGCCAGATGTCCTTAATCGCCTGAAGCCTGATAT |
| | | CATGATTGTTCATGGAGACAGGTTTGATGCCCTGGCTCTGGCCACATCTGCTGCCTTGATGAACA |
| | | TCCGAATCCTTCACATTGAAGGTGGGGAAGTCAGTGGGACCATTGATGACTCTATCAGACATGCC |
| | | ATAACAAAACTGGCTCATTATCATGTGTGCTGCACCCGCAGTGCAGAGCAGCACCTGATATCCAT |
| | | GTGTGAGGACCATGATCGCATCCTTTTGGCAGGCTGCCCTTCCTATGACAAACTTCTCTCAGCCA |
| | | AGAACAAAGACTACATGAGCATCATTCGCATGTGGCTAGGTGATGATGTAAAATCTAAAGATTAC |
| | | ATTGTTGCACTACAGCACCCTGTGACCACTGACATTAAGCATTCCATAAAAATGTTTGAATTAAC |
| | | ATTGGATGCACTTATCTCATTTAACAAGCGGACCCTAGTCCTGTTTCCAAATATTGACGCAGGGA |
| | | GCAAAGAGATGGTTCGAGTGATGCGGAAGAAGGGCATTGAGCATCATCCCAACTTTCGTGCAGTT |
| | | AAACACGTCCCATTTGACCAGTTTATACAGTTGGTTGCCCATGCTGGCTGTATGATTGGGAACAG |
| | | CAGCTGTGGGGTTCGAGAAGTTGGAGCTTTTGGAACACCTGTGATCAACCTGGGAACACGTCAGA |
| | | TTGGAAGAGAAACAGGGGAGAATGTTCTTCATGTCCGGGATGCTGACACCCAAGACAAAATATTG |
| | | CAAGCACTGCACCTTCAGTTTGGTAAACAGTACCCTTGTTCAAAGATATATGGGGATGGAAATGC |
| | | TGTTCCAAGGATTTTGAAGTTTCTCAAATCTATCGATCTTCAAGAGCCACTGCAAAAGAAATTCT |
| | | GCTTTCCTCCTGTGAAGGAGAATATCTCTCAAGATATTGACCATATTCTTGAAACTCTAAGTGCC |
| | | TTGGCCGTTGATCTTGGCGGGACGAACCTCCGAGTTGCAATAGTCAGCATGAAGGGTGAAATAGT |
| | | TAAGAAGTATACTCAGTTCAATCCTAAAACCTATGAAGAGAGGATTAATTTAATCCTACAGATGT |
| | | GTGTGGAAGCTGCAGCAGAAGCTGTAAAACTGAACTGCAGAATTTTGGGAGTAGGCATTTCCACA |
| | | GGTGGCCGTGTAAATCCTCGGGAAGGAATTGTGCTGCATTCAACCAAACTGATCCAAGAGTGGAA |
| | | CTCTGTGGACCTTAGGACCCCCCTTTCTGACACTTTGCATCTCCCTGTGTGGGTAGACAATGATG |
| | | GCAACTGTGCTGCCCTGGCGGAAAGGAAATTTGGCCAAGGAAAGGGACTGGAAAACTTTGTTACA |
| | | CTTATCACAGGCACAGGAATCGGTGGTGGAATTATCCATCAGCATGAATTGATCCACGGAAGCTC |
| | | CTTCTGTGCTGCAGAACTGGGCCACCTTGTTGTGTCTCTGGATGGGCCTGATTGTTCCTGTGGAA |
| | | GCCATGGGTGCATTGAAGCATACGCCTCTGGAATGGCCTTGCAGAGGGAGGCAAAAAAGCTCCAT |
| | | GATGAGGACCTGCTCTTGGTGGAAGGGATGTCAGTGCCAAAAGATGAGGCTGTGGGTGCGCTCCA |
| | | TCTCATCCAAGCTGCGAAACTTGGCAATGCGAAGGCCCAGAGCATCCTAAGAACAGCTGGAACAG |
| | | CTTTGGGTCTTGGGGTTGTGAACATCCTCCATACCATGAATCCCTCCCTTGTGATCCTCTCCGGA |
| | | GTCCTGGCCAGTCACTATATCCACATTGTCAAAGACGTCATTCGCCAGCAGGCCTTGTCCTCCGT |
| | | GCAGGACGTGGATGTGGTGGTTTCGGATTTGGTTGACCCCGCCCTGCTGGGTGCTGCCAGCATGG |
| | | TTCTGGACTACACAACACGCAGGATCTACTAGACCTCCAGGAACAGACATGGACCTTCTCTCCAG |
| | | AGCTCCTGAGTGGAATCAAGTTCTTGTCTTTAGGATGACCGTTTCTTAACAATCAAATCTGGTAT |
| | | TGAACTGCAGGTGACTTTGGCAGAGAAATGTTTTCACTTTTGGTCTCCTCTTCCAGAGTCACCTT |
| | | TCCCCACTCCTATTTTTGgttttccctctgccaaaaattatggggacatcatgaagccccttgag |
| | | catctgacttctggctaataaaggaaatttattttcattgcaatagtgtgttggaattttttgtg |
| | | tctctcactcggaaggacatatgggagggcaaatcatttaaaacatcagaatgagtatttggttt |
| | | agagtttggcaacatatgcccattcttccgcttcctcgctcactgactcgctgcgctcggtcgtt |
| | | cggctgcggcgagcggtatcagctcactcaaaggcggtaatacggttatccacagaatcagggga |
| | | taacgcaggaaagaacatgtgagcaaaaggccagcaaaaggccaggaaccgtaaaaaggccgcgt |
| | | tgctggcgtttttccataggctccgcccccctgacgagcatcacaaaaatcgacgctcaagtcag |
| | | aggtggcgaaacccgacaggactataaagataccaggcgtttccccctggaagctccctcgtgcg |
| | | ctctcctgttccgaccctgccgcttaccggatacctgtccgcctttctcccttcgggaagcgtgg |
| | | cgctttctcatagctcacgctgtaggtatctcagttcggtgtaggtcgttcgctccaagctgggc |
| | | tgtgtgcacgaaccccccgttcagcccgaccgctgcgccttatccggtaactatcgtcttgagtc |
| | | caacccggtaagacacgacttatcgccactggcagcagccactggtaacaggattagcagagcga |
| | | ggtatgtaggcggtgctacagagttcttgaagtggtggcctaactacggctacactagaagaaca |
| | | gtatttggtatctgcgctctgctgaagccagttaccttcggaaaaagagttggtagctcttgatc |
| | | cggcaaacaaaccaccgctggtagcggtggtttttttgtttgcaagcagcagattacgcgcagaa |
| | | aaaaaggatctcaagaagatcctttgatcttttctacggggtctgacgctcagtggaacgaaaac |
| | | tcacgttaagggattttggtcatgagattatcaaaaaggatcttcacctagatccttttaaatta |
| | | aaaatgaagttttaaatcaatctaaagtatatatgagtaaacttggtctgacagttaccaatgct |
| | | taatcagtgaggcacctatctcagcgatctgtctatttcgttcatccatagttgcctgactcggg |
| | | gggggggggcgctgaggtctgcctcgtgaagaaggtgttgctgactcataccaggcctgaatcgc |
| | | cccatcatccagccagaaagtgagggagccacggttgatgagagctttgttgtaggtggaccagt |
| | | tggtgattttgaacttttgctttgccacggaacggtctgcgttgtcgggaagatgcgtgatctga |
| | | tccttcaactcagcaaaagttcgatttattcaacaaagccgccgtcccgtcaagtcagcgtaatg |
| | | ctctgccagtgttacaaccaattaaccaattctgattagaaaaactcatcgagcatcaaatgaaa |
| | | ctgcaatttattcatatcaggattatcaataccatatttttgaaaaagccgtttctgtaatgaag |
| | | gagaaaactcaccgaggcagttccataggatggcaagatcctggtatcggtctgcgattccgact |
| | | cgtccaacatcaatacaacctattaatttcccctcgtcaaaaataaggttatcaagtgagaaatc |
| | | accatgagtgacgactgaatccggtgagaatggcaaaagcttatgcatttctttccagacttgtt |
| | | caacaggccagccattacgctcgtcatcaaaatcactcgcatcaaccaaaccgttattcattcgt |
| | | gattgcgcctgagcgagacgaaatacgcgatcgctgttaaaaggacaattacaaacaggaatcga |
| | | atgcaaccggcgcaggaacactgccagcgcatcaacaatattttcacctgaatcaggatattctt |
| | | ctaatacctggaatgctgttttcccggggatcgcagtggtgagtaaccatgcatcatcaggagta |
| | | cggataaaatgcttgatggtcggaagaggcataaattccgtcagccagtttagtctgaccatctc |
| | | atctgtaacatcattggcaacgctacctttgccatgtttcagaaacaactctggcgcatcgggct |
| | | tcccatacaatcgatagattgtcgcacctgattgcccgacattatcgcgagcccatttataccca |
| | | tataaatcagcatccatgttggaatttaatcgcggcctcgagcaagacgtttcccgttgaatatg |
| | | gctcataacaccccttgtattactgtttatgtaagcagacagttttattgttcatgatgatatat |
| | | ttttatcttgtgcaatgtaacatcagagattttgagacacaacgtggctttccccccccccccat |
| | | tattgaagcatttatcagggttattgtctcatgagcggatacatatttgaatgtatttagaaaaa |
| | | taaacaaataggggttccgcgcacatttccccgaaaagtgccacctgacgtctaagaaaccatta |
| | | ttatcatgacattaacctataaaaataggcgtatcacgaggccctttcgtctcgcgcgtttcggt |
| | | gatgacggtgaaaacctctgacacatgcagctcccggagacggtcacagcttgtctgtaagcgga |
| | | tgccgggagcagacaagcccgtcagggcgcgtcagcgggtgttggcgggtgtcggggctggctta |
| | | actatgcggcatcagagcagattgtactgagagtgcaccatatgcggtgtgaaataccgcacaga |
| | | tgcgtaaggagaaaataccgcatcagattggctat |
| 12 | Vector expressing | tggccattgcatacgttgtatccatatcataatatgtacatttatattggctcatgtccaacatt |
| | hGNE1 and | accgccatgttgacattgattattgactagttattaatagtaatcaattacggggtcattagttc |
| | knockdown three | atagcccatatatggagttccgcgttacataacttacggtaaatggcccgcctggctgaccgccc |
| | hGNE1 mutations in | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | | atatgccaagtacgccccctattgacgtcaatgacggtaaatggcccgcctggcattatgcccag |
| | | tacatgaccttatgggactttcctacttggcagtacatctacgtattagtcatcgctattaccat |
| | | ggtgatgcggttttggcagtacatcaatgggcgtggatagcggtttgactcacggggatttccaa |
| | | gtctccaccccattgacgtcaatgggagtttgttttggcaccaaaatcaacgggactttccaaaa |
| | | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacGTAAGTACCGCCTATAGACTCTATAGGCACACCCCTTTGGCTCTTATGCA |
| | | TGCTATACTGTTTTTGGCTTGGGGCCTATACACCCCCGCTTCCTTATGCTATAGGTGATGGTATA |
| | | GCTTAGCCTATAGGTGTGGGTTATTGACCATTATTGACCACTCCAACGGTGGAGGGCAGTGTAGT |
| | | CTGAGCAGTACTCGTTGCTGCCGCGCGCGCCACCAGACATAATAGCTGACAGACTAACAGACTGT |
| | | TCCTTTCCATGGGTCTTTTCTGCAGTCACCGTCGTCTACCCGCGCGCCCGAGCGCGGAAACCGAG |
| | | GGGCAGGCTCGCGCTCTGCCTGCTTCGTGGCGCTTGGTTCGTCCCTCGCCCGAGGAGCGCGGTGG |
| | | CGGCGTGGGAGGGAGCCTCTGACGGCGTCTGGAACTCTATTTTAAGAACCTCTCAAAACGAAACA |
| | | AGCAAATCATGGAGAAGAATGGAAATAACCGAAAGCTGCGGGTTTGTGTTGCTACTTGTAACCGT |
| | | GCAGATTATTCTAAACTTGCCCCGATCATGTTTGGCATTAAAACCGAACCTGAGTTCTTTGAACT |
| | | TGATGTTGTGGTACTTGGCTCTCACCTGATAGATGACTATGGAAATACATATCGAATGATTGAAC |
| | | AAGATGACTTTGACATTAACACCAGGCTACACACAATTGTGAGGGGAGAAGATGAGGCAGCCATG |
| | | GTGGAGTCAGTAGGCCTGGCCCTAGTGAAGCTGCCAGATGTCCTTAATCGCCTGAAGCCTGATAT |
| | | CATGATTGTTCATGGAGACAGGTTTGATGCCCTGGCTCTGGCCACATCTGCTGCCTTGATGAACA |
| | | TCCGAATCCTTCACATTGAAGGTGGGGAAGTCAGTGGGACCATTGATGACTCTATCAGACATGCC |
| | | ATAACAAAACTGGCTCATTATCATGTGTGCTGCACCCGCAGTGCAGAGCAGCACCTGATATCCAT |
| | | GTGTGAGGACCATGATCGCATCCTTTTGGCAGGCTGCCCTTCCTATGACAAACTTCTCTCAGCCA |
| | | AGAACAAAGACTACATGAGCATCATTCGCATGTGGCTAGGTGATGATGTAAAATCTAAAGATTAC |
| | | ATTGTTGCACTACAGCACCCTGTGACCACTGACATTAAGCATTCCATAAAAATGTTTGAATTAAC |
| | | ATTGGATGCACTTATCTCATTTAACAAGCGGACCCTAGTCCTGTTTCCAAATATTGACGCAGGGA |
| | | GCAAAGAGATGGTTCGAGTGATGCGGAAGAAGGGCATTGAGCATCATCCCAACTTTCGTGCAGTT |
| | | AAACACGTCCCATTTGACCAGTTTATACAGTTGGTTGCCCATGCTGGCTGTATGATTGGGAACAG |
| | | CAGCTGTGGGGTTCGAGAAGTTGGAGCTTTTGGAACACCTGTGATCAACCTGGGAACACGTCAGA |
| | | TTGGAAGAGAAACAGGGGAGAATGTTCTTCATGTCCGGGATGCTGACACCCAAGACAAAATATTG |
| | | CAAGCACTGCACCTTCAGTTTGGTAAACAGTACCCTTGTTCAAAGATATATGGGGATGGAAATGC |
| | | TGTTCCAAGGATTTTGAAGTTTCTCAAATCTATCGATCTTCAAGAGCCACTGCAAAAGAAATTCT |
| | | GCTTTCCTCCTGTGAAGGAGAATATCTCTCAAGATATTGACCATATTCTTGAAACTCTAAGTGCC |
| | | TTGGCCGTTGATCTTGGCGGGACGAACCTCCGAGTTGCAATAGTCAGCATGAAGGGTGAAATAGT |
| | | TAAGAAGTATACTCAGTTCAATCCTAAAACCTATGAAGAGAGGATTAATTTAATCCTACAGATGT |
| | | GTGTGGAAGCTGCAGCAGAAGCTGTAAAACTGAACTGCAGAATTTTGGGAGTAGGCATTTCCACA |
| | | GGTGGCCGTGTAAATCCTCGGGAAGGAATTGTGCTGCATTCAACCAAACTGATCCAAGAGTGGAA |
| | | CTCTGTGGACCTTAGGACCCCCCTTTCTGACACTTTGCATCTCCCTGTGTGGGTAGACAATGATG |
| | | GCAACTGTGCTGCCCTGGCGGAAAGGAAATTTGGCCAAGGAAAGGGACTGGAAAACTTTGTTACA |
| | | CTTATCACAGGCACAGGAATCGGTGGTGGAATTATCCATCAGCATGAATTGATCCACGGAAGCTC |
| | | CTTCTGTGCTGCAGAACTGGGCCACCTTGTTGTGTCTCTGGATGGGCCTGATTGTTCCTGTGGAA |
| | | GCCATGGGTGCATTGAAGCATACGCCTCTGGAATGGCCTTGCAGAGGGAGGCAAAAAAGCTCCAT |
| | | GATGAGGACCTGCTCTTGGTGGAAGGGATGTCAGTGCCAAAAGATGAGGCTGTGGGTGCGCTCCA |
| | | TCTCATCCAAGCTGCGAAACTTGGCAATGCGAAGGCCCAGAGCATCCTAAGAACAGCTGGAACAG |
| | | CTTTGGGTCTTGGGGTTGTGAACATCCTCCATACCATGAATCCCTCCCTTGTGATCCTCTCCGGA |
| | | GTCCTGGCCAGTCACTATATCCACATTGTCAAAGACGTCATTCGCCAGCAGGCCTTGTCCTCCGT |
| | | GCAGGACGTGGATGTGGTGGTTTCGGATTTGGTTGACCCCGCCCTGCTGGGTGCTGCCAGCATGG |
| | | TTCTGGACTACACAACACGCAGGATCTACTAGACCTCCAGGAACAGACATGGACCTTCTCTCCAG |
| | | AGCTCCTGAGTGGAATCAAGTTCTTGTCTTTAGGATGACCGTTTCTTAACAATCAAATCTGGTAT |
| | | TGAACTGCAGGTGACTTTGGCAGAGAAATGTTTTCACTTTTGGTCTCCTCTTCCAGAGTCACCTT |
| | | TCCCCACTCCTATTTTTGgTTTCTTCCCCATTAGGGATTATGCTGAATTTGTATGGTTTATAGTT |
| | | GTTAGAGTTTGAGGTGTTAATTCTAATTATCTATTTCAAATTTAGCAGGAAAAAAGAGAACATCA |
| | | CCTTGTAAAACTGAAGATTGTGACCAGTCAGAATAATGTCCTGCTGGGTGCTGCCAGCACGGAGA |
| | | TATGTGCATCTCCGTGCTGGCAGCACCCAGCAGGCATTATGGTGACAGCTGCCTCGGGAAGCCAA |
| | | GTTGGGCTTTAAAGTGCAGGGCCTGCTGATGTTGAGTGCTTTTTGTTCCATGCTGTGTATTGCCA |
| | | TCATCGAGTGAAGTAGATTAGCATCTCCGTGCTGGCAGCACCCAGCAGGCATAAGAAGTTATGTA |
| | | TTCATCCAATAATTCAAGCCAAGCAAGTATATAGGTGTTTTAATAGTTTTTGTTTGCAGTCCTCT |
| | | GTTATCCATGTGTGAGGACCATGTTCAGAAGAATGTACTGAACATGGTCCTCACACATGGATTGG |
| | | TGGCCTGCTATTTCCTTCAAATGAATGATTTTTACTAATTTTGTGTACTTTTATTGTGTCGATGT |
| | | AGAATCTGCCTGGTCTATCTGATGTGACAGCTTCTGTAGCACTATATCTGTGTACAGACTATGAT |
| | | CTGTTTAGTTATCAGAACATGGTCCTCACACATGGATTACTGCTAGCTGTAGAACTCCAGCTTCG |
| | | GCCTGTCGCCCAATCAAACTGTCCTGTTACTGAACACTGTTCTATGGTTCAGAACTGGGCCACCT |
| | | TGTTCTGGCTGTGATATTCTGCCAGAACAAGGTGGCCCAGTTCTGCTGTGGTAGTGAAAAGTCTG |
| | | TAGAAAAGTAAGGGAAACTCAAACCCCTTTCTACACATCACTGGTTAACATTGAATTGCCGTTTC |
| | | TGTATGGCAGAACAAGGTGGCCCAGTTCTGGAGTTTGGTGGGGATTGTGACCAGAAGATTTTGAA |
| | | AATTAAATATTACTGAAGATTTCGACTTCCACTGTTAAATGTACAAGATACATGAAATATTAAAG |
| | | AAAATGTGTAACTTTTTGTGTAAATACATCTTGTttttccctctgccaaaaattatggggacatc |
| | | atgaagccccttgagcatctgacttctggctaataaaggaaatttattttcattgcaatagtgtg |
| | | ttggaattttttgtgtctctcactcggaaggacatatgggagggcaaatcatttaaaacatcaga |
| | | atgagtatttggtttagagtttggcaacatatgcccattcttccgcttcctcgctcactgactcg |
| | | ctgcgctcggtcgttcggctgcggcgagcggtatcagctcactcaaaggcggtaatacggttatc |
| | | cacagaatcaggggataacgcaggaaagaacatgtgagcaaaaggccagcaaaaggccaggaacc |
| | | gtaaaaaggccgcgttgctggcgtttttccataggctccgcccccctgacgagcatcacaaaaat |
| | | cgacgctcaagtcagaggtggcgaaacccgacaggactataaagataccaggcgtttccccctgg |
| | | aagctccctcgtgcgctctcctgttccgaccctgccgcttaccggatacctgtccgcctttctcc |
| | | cttcgggaagcgtggcgctttctcatagctcacgctgtaggtatctcagttcggtgtaggtcgtt |
| | | cgctccaagctgggctgtgtgcacgaaccccccgttcagcccgaccgctgcgccttatccggtaa |
| | | ctatcgtcttgagtccaacccggtaagacacgacttatcgccactggcagcagccactggtaaca |
| | | ggattagcagagcgaggtatgtaggcggtgctacagagttcttgaagtggtggcctaactacggc |
| | | tacactagaagaacagtatttggtatctgcgctctgctgaagccagttaccttcggaaaaagagt |
| | | tggtagctcttgatccggcaaacaaaccaccgctggtagcggtggtttttttgtttgcaagcagc |
| | | agattacgcgcagaaaaaaaggatctcaagaagatcctttgatcttttctacggggtctgacgct |
| | | cagtggaacgaaaactcacgttaagggattttggtcatgagattatcaaaaaggatcttcaccta |
| | | gatccttttaaattaaaaatgaagttttaaatcaatctaaagtatatatgagtaaacttggtctg |
| | | acagttaccaatgcttaatcagtgaggcacctatctcagcgatctgtctatttcgttcatccata |
| | | gttgcctgactcggggggggggggcgctgaggtctgcctcgtgaagaaggtgttgctgactcata |
| | | ccaggcctgaatcgccccatcatccagccagaaagtgagggagccacggttgatgagagctttgt |
| | | tgtaggtggaccagttggtgattttgaacttttgctttgccacggaacggtctgcgttgtcggga |
| | | agatgcgtgatctgatccttcaactcagcaaaagttcgatttattcaacaaagccgccgtcccgt |
| | | caagtcagcgtaatgctctgccagtgttacaaccaattaaccaattctgattagaaaaactcatc |
| | | gagcatcaaatgaaactgcaatttattcatatcaggattatcaataccatatttttgaaaaagcc |
| | | gtttctgtaatgaaggagaaaactcaccgaggcagttccataggatggcaagatcctggtatcgg |
| | | tctgcgattccgactcgtccaacatcaatacaacctattaatttcccctcgtcaaaaataaggtt |
| | | atcaagtgagaaatcaccatgagtgacgactgaatccggtgagaatggcaaaagcttatgcattt |
| | | ctttccagacttgttcaacaggccagccattacgctcgtcatcaaaatcactcgcatcaaccaaa |
| | | ccgttattcattcgtgattgcgcctgagcgagacgaaatacgcgatcgctgttaaaaggacaatt |
| | | acaaacaggaatcgaatgcaaccggcgcaggaacactgccagcgcatcaacaatattttcacctg |
| | | aatcaggatattcttctaatacctggaatgctgttttcccggggatcgcagtggtgagtaaccat |
| | | gcatcatcaggagtacggataaaatgcttgatggtcggaagaggcataaattccgtcagccagtt |
| | | tagtctgaccatctcatctgtaacatcattggcaacgctacctttgccatgtttcagaaacaact |
| | | ctggcgcatcgggcttcccatacaatcgatagattgtcgcacctgattgcccgacattatcgcga |
| | | gcccatttatacccatataaatcagcatccatgttggaatttaatcgcggcctcgagcaagacgt |
| | | ttcccgttgaatatggctcataacaccccttgtattactgtttatgtaagcagacagttttattg |
| | | ttcatgatgatatatttttatcttgtgcaatgtaacatcagagattttgagacacaacgtggctt |
| | | tccccccccccccattattgaagcatttatcagggttattgtctcatgagcggatacatatttga |
| | | atgtatttagaaaaataaacaaataggggttccgcgcacatttccccgaaaagtgccacctgacg |
| | | tctaagaaaccattattatcatgacattaacctataaaaataggcgtatcacgaggccctttcgt |
| | | ctcgcgcgtttcggtgatgacggtgaaaacctctgacacatgcagctcccggagacggtcacagc |
| | | ttgtctgtaagcggatgccgggagcagacaagcccgtcagggcgcgtcagcgggtgttggcgggt |
| | | gtcggggctggcttaactatgcggcatcagagcagattgtactgagagtgcaccatatgcggtgt |
| | | gaaataccgcacagatgcgtaaggagaaaataccgcatcagattggctat |
| 13 | Vector expressing | tggccattgcatacgttgtatccatatcataatatgtacatttatattggctcatgtccaacatt |
| | hGNE1 and | accgccatgttgacattgattattgactagttattaatagtaatcaattacggggtcattagttc |
| | knockdown D176V | atagcccatatatggagttccgcgttacataacttacggtaaatggcccgcctggctgaccgccc |
| | hGNE1 mutations in | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | | atatgccaagtacgccccctattgacgtcaatgacggtaaatggcccgcctggcattatgcccag |
| | | tacatgaccttatgggactttcctacttggcagtacatctacgtattagtcatcgctattaccat |
| | | ggtgatgcggttttggcagtacatcaatgggcgtggatagcggtttgactcacggggatttccaa |
| | | gtctccaccccattgacgtcaatgggagtttgttttggcaccaaaatcaacgggactttccaaaa |
| | | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacGTAAGTACCGCCTATAGACTCTATAGGCACACCCCTTTGGCTCTTATGCA |
| | | TGCTATACTGTTTTTGGCTTGGGGCCTATACACCCCCGCTTCCTTATGCTATAGGTGATGGTATA |
| | | GCTTAGCCTATAGGTGTGGGTTATTGACCATTATTGACCACTCCAACGGTGGAGGGCAGTGTAGT |
| | | CTGAGCAGTACTCGTTGCTGCCGCGCGCGCCACCAGACATAATAGCTGACAGACTAACAGACTGT |
| | | TCCTTTCCATGGGTCTTTTCTGCAGTCACCGTCGTCTACCCGCGCGCCCGAGCGCGGAAACCGAG |
| | | GGGCAGGCTCGCGCTCTGCCTGCTTCGTGGCGCTTGGTTCGTCCCTCGCCCGAGGAGCGCGGTGG |
| | | CGGCGTGGGAGGGAGCCTCTGACGGCGTCTGGAACTCTATTTTAAGAACCTCTCAAAACGAAACA |
| | | AGCAAATCATGGAGAAGAATGGAAATAACCGAAAGCTGCGGGTTTGTGTTGCTACTTGTAACCGT |
| | | GCAGATTATTCTAAACTTGCCCCGATCATGTTTGGCATTAAAACCGAACCTGAGTTCTTTGAACT |
| | | TGATGTTGTGGTACTTGGCTCTCACCTGATAGATGACTATGGAAATACATATCGAATGATTGAAC |
| | | AAGATGACTTTGACATTAACACCAGGCTACACACAATTGTGAGGGGAGAAGATGAGGCAGCCATG |
| | | GTGGAGTCAGTAGGCCTGGCCCTAGTGAAGCTGCCAGATGTCCTTAATCGCCTGAAGCCTGATAT |
| | | CATGATTGTTCATGGAGACAGGTTTGATGCCCTGGCTCTGGCCACATCTGCTGCCTTGATGAACA |
| | | TCCGAATCCTTCACATTGAAGGTGGGGAAGTCAGTGGGACCATTGATGACTCTATCAGACATGCC |
| | | ATAACAAAACTGGCTCATTATCATGTGTGCTGCACCCGCAGTGCAGAGCAGCACCTGATATCCAT |
| | | GTGTGAGGACCATGATCGCATCCTTTTGGCAGGCTGCCCTTCCTATGACAAACTTCTCTCAGCCA |
| | | AGAACAAAGACTACATGAGCATCATTCGCATGTGGCTAGGTGATGATGTAAAATCTAAAGATTAC |
| | | ATTGTTGCACTACAGCACCCTGTGACCACTGACATTAAGCATTCCATAAAAATGTTTGAATTAAC |
| | | ATTGGATGCACTTATCTCATTTAACAAGCGGACCCTAGTCCTGTTTCCAAATATTGACGCAGGGA |
| | | GCAAAGAGATGGTTCGAGTGATGCGGAAGAAGGGCATTGAGCATCATCCCAACTTTCGTGCAGTT |
| | | AAACACGTCCCATTTGACCAGTTTATACAGTTGGTTGCCCATGCTGGCTGTATGATTGGGAACAG |
| | | CAGCTGTGGGGTTCGAGAAGTTGGAGCTTTTGGAACACCTGTGATCAACCTGGGAACACGTCAGA |
| | | TTGGAAGAGAAACAGGGGAGAATGTTCTTCATGTCCGGGATGCTGACACCCAAGACAAAATATTG |
| | | CAAGCACTGCACCTTCAGTTTGGTAAACAGTACCCTTGTTCAAAGATATATGGGGATGGAAATGC |
| | | TGTTCCAAGGATTTTGAAGTTTCTCAAATCTATCGATCTTCAAGAGCCACTGCAAAAGAAATTCT |
| | | GCTTTCCTCCTGTGAAGGAGAATATCTCTCAAGATATTGACCATATTCTTGAAACTCTAAGTGCC |
| | | TTGGCCGTTGATCTTGGCGGGACGAACCTCCGAGTTGCAATAGTCAGCATGAAGGGTGAAATAGT |
| | | TAAGAAGTATACTCAGTTCAATCCTAAAACCTATGAAGAGAGGATTAATTTAATCCTACAGATGT |
| | | GTGTGGAAGCTGCAGCAGAAGCTGTAAAACTGAACTGCAGAATTTTGGGAGTAGGCATTTCCACA |
| | | GGTGGCCGTGTAAATCCTCGGGAAGGAATTGTGCTGCATTCAACCAAACTGATCCAAGAGTGGAA |
| | | CTCTGTGGACCTTAGGACCCCCCTTTCTGACACTTTGCATCTCCCTGTGTGGGTAGACAATGATG |
| | | GCAACTGTGCTGCCCTGGCGGAAAGGAAATTTGGCCAAGGAAAGGGACTGGAAAACTTTGTTACA |
| | | CTTATCACAGGCACAGGAATCGGTGGTGGAATTATCCATCAGCATGAATTGATCCACGGAAGCTC |
| | | CTTCTGTGCTGCAGAACTGGGCCACCTTGTTGTGTCTCTGGATGGGCCTGATTGTTCCTGTGGAA |
| | | GCCATGGGTGCATTGAAGCATACGCCTCTGGAATGGCCTTGCAGAGGGAGGCAAAAAAGCTCCAT |
| | | GATGAGGACCTGCTCTTGGTGGAAGGGATGTCAGTGCCAAAAGATGAGGCTGTGGGTGCGCTCCA |
| | | TCTCATCCAAGCTGCGAAACTTGGCAATGCGAAGGCCCAGAGCATCCTAAGAACAGCTGGAACAG |
| | | CTTTGGGTCTTGGGGTTGTGAACATCCTCCATACCATGAATCCCTCCCTTGTGATCCTCTCCGGA |
| | | GTCCTGGCCAGTCACTATATCCACATTGTCAAAGACGTCATTCGCCAGCAGGCCTTGTCCTCCGT |
| | | GCAGGACGTGGATGTGGTGGTTTCGGATTTGGTTGACCCCGCCCTGCTGGGTGCTGCCAGCATGG |
| | | TTCTGGACTACACAACACGCAGGATCTACTAGACCTCCAGGAACAGACATGGACCTTCTCTCCAG |
| | | AGCTCCTGAGTGGAATCAAGTTCTTGTCTTTAGGATGACCGTTTCTTAACAATCAAATCTGGTAT |
| | | TGAACTGCAGGTGACTTTGGCAGAGAAATGTTTTCACTTTTGGTCTCCTCTTCCAGAGTCACCTT |
| | | TCCCCACTCCTATTTTTGgTTTCTTCCCCATTAGGGATTATGCTGAATTTGTATGGTTTATAGTT |
| | | GTTAGAGTTTGAGGTGTTAATTCTAATTATCTATTTCAAATTTAGCAGGAAAAAAGAGAACATCA |
| | | CCTTGTAAAACTGAAGATTGTGACCAGCAGTCCTCTGTTATCCATGTGTGAGGACCATGTTCAGA |
| | | AGAATGTACTGAACATGGTCCTCACACATGGATTGGTGGCCTGCTATTTCCTTCAAATGAATGAT |
| | | TTTTACTAATTTTGTGTACTTTTATTGTGTCGATGTAGAATCTGCCTGGTCTATCTGATGTGACA |
| | | GCTTCTGTAGCACTATATCTGTGTACAGACTATGATCTGTTTAGTTATCAGAACATGGTCCTCAC |
| | | ACATGGATTACTGCTAGCTGTAGAACTCCAGCTTCGGCCTGTCGCCCAATCAAACTGTCCTGTTA |
| | | CTGAATGGGGATTGTGACCAGAAGATTTTGAAAATTAAATATTACTGAAGATTTCGACTTCCACT |
| | | GTTAAATGTACAAGATACATGAAATATTAAAGAAAATGTGTAACTTTTTGTGTAAATACATCTTG |
| | | Tttttccctctgccaaaaattatggggacatcatgaagccccttgagcatctgacttctggctaa |
| | | taaaggaaatttattttcattgcaatagtgtgttggaattttttgtgtctctcactcggaaggac |
| | | atatgggagggcaaatcatttaaaacatcagaatgagtatttggtttagagtttggcaacatatg |
| | | cccattcttccgcttcctcgctcactgactcgctgcgctcggtcgttcggctgcggcgagcggta |
| | | tcagctcactcaaaggcggtaatacggttatccacagaatcaggggataacgcaggaaagaacat |
| | | gtgagcaaaaggccagcaaaaggccaggaaccgtaaaaaggccgcgttgctggcgtttttccata |
| | | ggctccgcccccctgacgagcatcacaaaaatcgacgctcaagtcagaggtggcgaaacccgaca |
| | | ggactataaagataccaggcgtttccccctggaagctccctcgtgcgctctcctgttccgaccct |
| | | gccgcttaccggatacctgtccgcctttctcccttcgggaagcgtggcgctttctcatagctcac |
| | | gctgtaggtatctcagttcggtgtaggtcgttcgctccaagctgggctgtgtgcacgaacccccc |
| | | gttcagcccgaccgctgcgccttatccggtaactatcgtcttgagtccaacccggtaagacacga |
| | | cttatcgccactggcagcagccactggtaacaggattagcagagcgaggtatgtaggcggtgcta |
| | | cagagttcttgaagtggtggcctaactacggctacactagaagaacagtatttggtatctgcgct |
| | | ctgctgaagccagttaccttcggaaaaagagttggtagctcttgatccggcaaacaaaccaccgc |
| | | tggtagcggtggtttttttgtttgcaagcagcagattacgcgcagaaaaaaaggatctcaagaag |
| | | atcctttgatcttttctacggggtctgacgctcagtggaacgaaaactcacgttaagggattttg |
| | | gtcatgagattatcaaaaaggatcttcacctagatccttttaaattaaaaatgaagttttaaatc |
| | | aatctaaagtatatatgagtaaacttggtctgacagttaccaatgcttaatcagtgaggcaccta |
| | | tctcagcgatctgtctatttcgttcatccatagttgcctgactcggggggggggggcgctgaggt |
| | | ctgcctcgtgaagaaggtgttgctgactcataccaggcctgaatcgccccatcatccagccagaa |
| | | agtgagggagccacggttgatgagagctttgttgtaggtggaccagttggtgattttgaactttt |
| | | gctttgccacggaacggtctgcgttgtcgggaagatgcgtgatctgatccttcaactcagcaaaa |
| | | gttcgatttattcaacaaagccgccgtcccgtcaagtcagcgtaatgctctgccagtgttacaac |
| | | caattaaccaattctgattagaaaaactcatcgagcatcaaatgaaactgcaatttattcatatc |
| | | aggattatcaataccatatttttgaaaaagccgtttctgtaatgaaggagaaaactcaccgaggc |
| | | agttccataggatggcaagatcctggtatcggtctgcgattccgactcgtccaacatcaatacaa |
| | | cctattaatttcccctcgtcaaaaataaggttatcaagtgagaaatcaccatgagtgacgactga |
| | | atccggtgagaatggcaaaagcttatgcatttctttccagacttgttcaacaggccagccattac |
| | | gctcgtcatcaaaatcactcgcatcaaccaaaccgttattcattcgtgattgcgcctgagcgaga |
| | | cgaaatacgcgatcgctgttaaaaggacaattacaaacaggaatcgaatgcaaccggcgcaggaa |
| | | cactgccagcgcatcaacaatattttcacctgaatcaggatattcttctaatacctggaatgctg |
| | | ttttcccggggatcgcagtggtgagtaaccatgcatcatcaggagtacggataaaatgcttgatg |
| | | gtcggaagaggcataaattccgtcagccagtttagtctgaccatctcatctgtaacatcattggc |
| | | aacgctacctttgccatgtttcagaaacaactctggcgcatcgggcttcccatacaatcgataga |
| | | ttgtcgcacctgattgcccgacattatcgcgagcccatttatacccatataaatcagcatccatg |
| | | ttggaatttaatcgcggcctcgagcaagacgtttcccgttgaatatggctcataacaccccttgt |
| | | attactgtttatgtaagcagacagttttattgttcatgatgatatatttttatcttgtgcaatgt |
| | | aacatcagagattttgagacacaacgtggctttccccccccccccattattgaagcatttatcag |
| | | ggttattgtctcatgagcggatacatatttgaatgtatttagaaaaataaacaaataggggttcc |
| | | gcgcacatttccccgaaaagtgccacctgacgtctaagaaaccattattatcatgacattaacct |
| | | ataaaaataggcgtatcacgaggccctttcgtctcgcgcgtttcggtgatgacggtgaaaacctc |
| | | tgacacatgcagctcccggagacggtcacagcttgtctgtaagcggatgccgggagcagacaagc |
| | | ccgtcagggcgcgtcagcgggtgttggcgggtgtcggggctggcttaactatgcggcatcagagc |
| | | agattgtactgagagtgcaccatatgcggtgtgaaataccgcacagatgcgtaaggagaaaatac |
| | | cgcatcagattggctat |
| 14 | Vector expressing | tggccattgcatacgttgtatccatatcataatatgtacatttatattggctcatgtccaacatt |
| | hGNE1 and | accgccatgttgacattgattattgactagttattaatagtaatcaattacggggtcattagttc |
| | knockdown V572L | atagcccatatatggagttccgcgttacataacttacggtaaatggcccgcctggctgaccgccc |
| | hGNE1 mutations in | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | | atatgccaagtacgccccctattgacgtcaatgacggtaaatggcccgcctggcattatgcccag |
| | | tacatgaccttatgggactttcctacttggcagtacatctacgtattagtcatcgctattaccat |
| | | ggtgatgcggttttggcagtacatcaatgggcgtggatagcggtttgactcacggggatttccaa |
| | | gtctccaccccattgacgtcaatgggagtttgttttggcaccaaaatcaacgggactttccaaaa |
| | | tgtgtaacaactccgccccattgacgcaaatgggcggtaggcgtgtacggtgggaggtctatata |
| | | agcagagctcgtttagtgaaccgtcagatcgcctggagacgccatccacgctgttttgacctcca |
| | | tagaagacaccgggaccgatccagcctccgcggccgggaacggtgcattggaacgcggattcccc |
| | | gtgccaagagtgacGTAAGTACCGCCTATAGACTCTATAGGCACACCCCTTTGGCTCTTATGCAT |
| | | GCTATACTGTTTTTGGCTTGGGGCCTATACACCCCCGCTTCCTTATGCTATAGGTGATGGTATAG |
| | | CTTAGCCTATAGGTGTGGGTTATTGACCATTATTGACCACTCCAACGGTGGAGGGCAGTGTAGTC |
| | | TGAGCAGTACTCGTTGCTGCCGCGCGCGCCACCAGACATAATAGCTGACAGACTAACAGACTGTT |
| | | CCTTTCCATGGGTCTTTTCTGCAGTCACCGTCGTCTACCCGCGCGCCCGAGCGCGGAAACCGAGG |
| | | GGCAGGCTCGCGCTCTGCCTGCTTCGTGGCGCTTGGTTCGTCCCTCGCCCGAGGAGCGCGGTGGC |
| | | GGCGTGGGAGGGAGCCTCTGACGGCGTCTGGAACTCTATTTTAAGAACCTCTCAAAACGAAACAA |
| | | GCAAATCATGGAGAAGAATGGAAATAACCGAAAGCTGCGGGTTTGTGTTGCTACTTGTAACCGTG |
| | | CAGATTATTCTAAACTTGCCCCGATCATGTTTGGCATTAAAACCGAACCTGAGTTCTTTGAACTT |
| | | GATGTTGTGGTACTTGGCTCTCACCTGATAGATGACTATGGAAATACATATCGAATGATTGAACA |
| | | AGATGACTTTGACATTAACACCAGGCTACACACAATTGTGAGGGGAGAAGATGAGGCAGCCATGG |
| | | TGGAGTCAGTAGGCCTGGCCCTAGTGAAGCTGCCAGATGTCCTTAATCGCCTGAAGCCTGATATC |
| | | ATGATTGTTCATGGAGACAGGTTTGATGCCCTGGCTCTGGCCACATCTGCTGCCTTGATGAACAT |
| | | CCGAATCCTTCACATTGAAGGTGGGGAAGTCAGTGGGACCATTGATGACTCTATCAGACATGCCA |
| | | TAACAAAACTGGCTCATTATCATGTGTGCTGCACCCGCAGTGCAGAGCAGCACCTGATATCCATG |
| | | TGTGAGGACCATGATCGCATCCTTTTGGCAGGCTGCCCTTCCTATGACAAACTTCTCTCAGCCAA |
| | | GAACAAAGACTACATGAGCATCATTCGCATGTGGCTAGGTGATGATGTAAAATCTAAAGATTACA |
| | | TTGTTGCACTACAGCACCCTGTGACCACTGACATTAAGCATTCCATAAAAATGTTTGAATTAACA |
| | | TTGGATGCACTTATCTCATTTAACAAGCGGACCCTAGTCCTGTTTCCAAATATTGACGCAGGGAG |
| | | CAAAGAGATGGTTCGAGTGATGCGGAAGAAGGGCATTGAGCATCATCCCAACTTTCGTGCAGTTA |
| | | AACACGTCCCATTTGACCAGTTTATACAGTTGGTTGCCCATGCTGGCTGTATGATTGGGAACAGC |
| | | AGCTGTGGGGTTCGAGAAGTTGGAGCTTTTGGAACACCTGTGATCAACCTGGGAACACGTCAGAT |
| | | TGGAAGAGAAACAGGGGAGAATGTTCTTCATGTCCGGGATGCTGACACCCAAGACAAAATATTGC |
| | | AAGCACTGCACCTTCAGTTTGGTAAACAGTACCCTTGTTCAAAGATATATGGGGATGGAAATGCT |
| | | GTTCCAAGGATTTTGAAGTTTCTCAAATCTATCGATCTTCAAGAGCCACTGCAAAAGAAATTCTG |
| | | CTTTCCTCCTGTGAAGGAGAATATCTCTCAAGATATTGACCATATTCTTGAAACTCTAAGTGCCT |
| | | TGGCCGTTGATCTTGGCGGGACGAACCTCCGAGTTGCAATAGTCAGCATGAAGGGTGAAATAGTT |
| | | AAGAAGTATACTCAGTTCAATCCTAAAACCTATGAAGAGAGGATTAATTTAATCCTACAGATGTG |
| | | TGTGGAAGCTGCAGCAGAAGCTGTAAAACTGAACTGCAGAATTTTGGGAGTAGGCATTTCCACAG |
| | | GTGGCCGTGTAAATCCTCGGGAAGGAATTGTGCTGCATTCAACCAAACTGATCCAAGAGTGGAAC |
| | | TCTGTGGACCTTAGGACCCCCCTTTCTGACACTTTGCATCTCCCTGTGTGGGTAGACAATGATGG |
| | | CAACTGTGCTGCCCTGGCGGAAAGGAAATTTGGCCAAGGAAAGGGACTGGAAAACTTTGTTACAC |
| | | TTATCACAGGCACAGGAATCGGTGGTGGAATTATCCATCAGCATGAATTGATCCACGGAAGCTCC |
| | | TTCTGTGCTGCAGAACTGGGCCACCTTGTTGTGTCTCTGGATGGGCCTGATTGTTCCTGTGGAAG |
| | | CCATGGGTGCATTGAAGCATACGCCTCTGGAATGGCCTTGCAGAGGGAGGCAAAAAAGCTCCATG |
| | | ATGAGGACCTGCTCTTGGTGGAAGGGATGTCAGTGCCAAAAGATGAGGCTGTGGGTGCGCTCCAT |
| | | CTCATCCAAGCTGCGAAACTTGGCAATGCGAAGGCCCAGAGCATCCTAAGAACAGCTGGAACAGC |
| | | TTTGGGTCTTGGGGTTGTGAACATCCTCCATACCATGAATCCCTCCCTTGTGATCCTCTCCGGAG |
| | | TCCTGGCCAGTCACTATATCCACATTGTCAAAGACGTCATTCGCCAGCAGGCCTTGTCCTCCGTG |
| | | CAGGACGTGGATGTGGTGGTTTCGGATTTGGTTGACCCCGCCCTGCTGGGTGCTGCCAGCATGGT |
| | | TCTGGACTACACAACACGCAGGATCTACTAGACCTCCAGGAACAGACATGGACCTTCTCTCCAGA |
| | | GCTCCTGAGTGGAATCAAGTTCTTGTCTTTAGGATGACCGTTTCTTAACAATCAAATCTGGTATT |
| | | GAACTGCAGGTGACTTTGGCAGAGAAATGTTTTCACTTTTGGTCTCCTCTTCCAGAGTCACCTTT |
| | | CCCCACTCCTATTTTTGgTTTCTTCCCCATTAGGGATTATGCTGAATTTGTATGGTTTATAGTTG |
| | | TTAGAGTTTGAGGTGTTAATTCTAATTATCTATTTCAAATTTAGCAGGAAAAAAGAGAACATCAC |
| | | CTTGTAAAACTGAAGATTGTGACCATAGCTGTAGAACTCCAGCTTCGGCCTGTCGCCCAATCAAA |
| | | CTGTCCTGTTACTGAACACTGTTCTATGGTTCAGAACTGGGCCACCTTGTTCTGGCTGTGATATT |
| | | CTGCCAGAACAAGGTGGCCCAGTTCTGCTGTGGTAGTGAAAAGTCTGTAGAAAAGTAAGGGAAAC |
| | | TCAAACCCCTTTCTACACATCACTGGTTAACATTGAATTGCCGTTTCTGTATGGCAGAACAAGGT |
| | | GGCCCAGTTCTGGAGTTTGGTGGGGATTGTGACCAGAAGATTTTGAAAATTAAATATTACTGAAG |
| | | ATTTCGACTTCCACTGTTAAATGTACAAGATACATGAAATATTAAAGAAAATGTGTAACTTTTTG |
| | | TGTAAATACATCTTGTttttccctctgccaaaaattatggggacatcatgaagccccttgagcat |
| | | ctgacttctggctaataaaggaaatttattttcattgcaatagtgtgttggaattttttgtgtct |
| | | ctcactcggaaggacatatgggagggcaaatcatttaaaacatcagaatgagtatttggtttaga |
| | | gtttggcaacatatgcccattcttccgcttcctcgctcactgactcgctgcgctcggtcgttcgg |
| | | ctgcggcgagcggtatcagctcactcaaaggcggtaatacggttatccacagaatcaggggataa |
| | | cgcaggaaagaacatgtgagcaaaaggccagcaaaaggccaggaaccgtaaaaaggccgcgttgc |
| | | tggcgtttttccataggctccgcccccctgacgagcatcacaaaaatcgacgctcaagtcagagg |
| | | tggcgaaacccgacaggactataaagataccaggcgtttccccctggaagctccctcgtgcgctc |
| | | tcctgttccgaccctgccgcttaccggatacctgtccgcctttctcccttcgggaagcgtggcgc |
| | | tttctcatagctcacgctgtaggtatctcagttcggtgtaggtcgttcgctccaagctgggctgt |
| | | gtgcacgaaccccccgttcagcccgaccgctgcgccttatccggtaactatcgtcttgagtccaa |
| | | cccggtaagacacgacttatcgccactggcagcagccactggtaacaggattagcagagcgaggt |
| | | atgtaggcggtgctacagagttcttgaagtggtggcctaactacggctacactagaagaacagta |
| | | tttggtatctgcgctctgctgaagccagttaccttcggaaaaagagttggtagctcttgatccgg |
| | | caaacaaaccaccgctggtagcggtggtttttttgtttgcaagcagcagattacgcgcagaaaaa |
| | | aaggatctcaagaagatcctttgatcttttctacggggtctgacgctcagtggaacgaaaactca |
| | | cgttaagggattttggtcatgagattatcaaaaaggatcttcacctagatccttttaaattaaaa |
| | | atgaagttttaaatcaatctaaagtatatatgagtaaacttggtctgacagttaccaatgcttaa |
| | | tcagtgaggcacctatctcagcgatctgtctatttcgttcatccatagttgcctgactcgggggg |
| | | ggggggcgctgaggtctgcctcgtgaagaaggtgttgctgactcataccaggcctgaatcgcccc |
| | | atcatccagccagaaagtgagggagccacggttgatgagagctttgttgtaggtggaccagttgg |
| | | tgattttgaacttttgctttgccacggaacggtctgcgttgtcgggaagatgcgtgatctgatcc |
| | | ttcaactcagcaaaagttcgatttattcaacaaagccgccgtcccgtcaagtcagcgtaatgctc |
| | | tgccagtgttacaaccaattaaccaattctgattagaaaaactcatcgagcatcaaatgaaactg |
| | | caatttattcatatcaggattatcaataccatatttttgaaaaagccgtttctgtaatgaaggag |
| | | aaaactcaccgaggcagttccataggatggcaagatcctggtatcggtctgcgattccgactcgt |
| | | ccaacatcaatacaacctattaatttcccctcgtcaaaaataaggttatcaagtgagaaatcacc |
| | | atgagtgacgactgaatccggtgagaatggcaaaagcttatgcatttctttccagacttgttcaa |
| | | caggccagccattacgctcgtcatcaaaatcactcgcatcaaccaaaccgttattcattcgtgat |
| | | tgcgcctgagcgagacgaaatacgcgatcgctgttaaaaggacaattacaaacaggaatcgaatg |
| | | caaccggcgcaggaacactgccagcgcatcaacaatattttcacctgaatcaggatattcttcta |
| | | atacctggaatgctgttttcccggggatcgcagtggtgagtaaccatgcatcatcaggagtacgg |
| | | ataaaatgcttgatggtcggaagaggcataaattccgtcagccagtttagtctgaccatctcatc |
| | | tgtaacatcattggcaacgctacctttgccatgtttcagaaacaactctggcgcatcgggcttcc |
| | | catacaatcgatagattgtcgcacctgattgcccgacattatcgcgagcccatttatacccatat |
| | | aaatcagcatccatgttggaatttaatcgcggcctcgagcaagacgtttcccgttgaatatggct |
| | | cataacaccccttgtattactgtttatgtaagcagacagttttattgttcatgatgatatatttt |
| | | tatcttgtgcaatgtaacatcagagattttgagacacaacgtggctttccccccccccccattat |
| | | tgaagcatttatcagggttattgtctcatgagcggatacatatttgaatgtatttagaaaaataa |
| | | acaaataggggttccgcgcacatttccccgaaaagtgccacctgacgtctaagaaaccattatta |
| | | tcatgacattaacctataaaaataggcgtatcacgaggccctttcgtctcgcgcgtttcggtgat |
| | | gacggtgaaaacctctgacacatgcagctcccggagacggtcacagcttgtctgtaagcggatgc |
| | | cgggagcagacaagcccgtcagggcgcgtcagcgggtgttggcgggtgtcggggctggcttaact |
| | | atgcggcatcagagcagattgtactgagagtgcaccatatgcggtgtgaaataccgcacagatgc |
| | | gtaaggagaaaataccgcatcagattggctat |
| 15 | Vector expressing | tggccattgcatacgttgtatccatatcataatatgtacatttatattggctcatgtccaacatt |
| | hGNE1 and | accgccatgttgacattgattattgactagttattaatagtaatcaattacggggtcattagttc |
| | knockdown M712T | atagcccatatatggagttccgcgttacataacttacggtaaatggcccgcctggctgaccgccc |
| | hGNE1 mutations in | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | | atatgccaagtacgccccctattgacgtcaatgacggtaaatggcccgcctggcattatgcccag |
| | | tacatgaccttatgggactttcctacttggcagtacatctacgtattagtcatcgctattaccat |
| | | ggtgatgcggttttggcagtacatcaatgggcgtggatagcggtttgactcacggggatttccaa |
| | | gtctccaccccattgacgtcaatgggagtttgttttggcaccaaaatcaacgggactttccaaaa |
| | | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacGTAAGTACCGCCTATAGACTCTATAGGCACACCCCTTTGGCTCTTATGCA |
| | | TGCTATACTGTTTTTGGCTTGGGGCCTATACACCCCCGCTTCCTTATGCTATAGGTGATGGTATA |
| | | GCTTAGCCTATAGGTGTGGGTTATTGACCATTATTGACCACTCCAACGGTGGAGGGCAGTGTAGT |
| | | CTGAGCAGTACTCGTTGCTGCCGCGCGCGCCACCAGACATAATAGCTGACAGACTAACAGACTGT |
| | | TCCTTTCCATGGGTCTTTTCTGCAGTCACCGTCGTCTACCCGCGCGCCCGAGCGCGGAAACCGAG |
| | | GGGCAGGCTCGCGCTCTGCCTGCTTCGTGGCGCTTGGTTCGTCCCTCGCCCGAGGAGCGCGGTGG |
| | | CGGCGTGGGAGGGAGCCTCTGACGGCGTCTGGAACTCTATTTTAAGAACCTCTCAAAACGAAACA |
| | | AGCAAATCATGGAGAAGAATGGAAATAACCGAAAGCTGCGGGTTTGTGTTGCTACTTGTAACCGT |
| | | GCAGATTATTCTAAACTTGCCCCGATCATGTTTGGCATTAAAACCGAACCTGAGTTCTTTGAACT |
| | | TGATGTTGTGGTACTTGGCTCTCACCTGATAGATGACTATGGAAATACATATCGAATGATTGAAC |
| | | AAGATGACTTTGACATTAACACCAGGCTACACACAATTGTGAGGGGAGAAGATGAGGCAGCCATG |
| | | GTGGAGTCAGTAGGCCTGGCCCTAGTGAAGCTGCCAGATGTCCTTAATCGCCTGAAGCCTGATAT |
| | | CATGATTGTTCATGGAGACAGGTTTGATGCCCTGGCTCTGGCCACATCTGCTGCCTTGATGAACA |
| | | TCCGAATCCTTCACATTGAAGGTGGGGAAGTCAGTGGGACCATTGATGACTCTATCAGACATGCC |
| | | ATAACAAAACTGGCTCATTATCATGTGTGCTGCACCCGCAGTGCAGAGCAGCACCTGATATCCAT |
| | | GTGTGAGGACCATGATCGCATCCTTTTGGCAGGCTGCCCTTCCTATGACAAACTTCTCTCAGCCA |
| | | AGAACAAAGACTACATGAGCATCATTCGCATGTGGCTAGGTGATGATGTAAAATCTAAAGATTAC |
| | | ATTGTTGCACTACAGCACCCTGTGACCACTGACATTAAGCATTCCATAAAAATGTTTGAATTAAC |
| | | ATTGGATGCACTTATCTCATTTAACAAGCGGACCCTAGTCCTGTTTCCAAATATTGACGCAGGGA |
| | | GCAAAGAGATGGTTCGAGTGATGCGGAAGAAGGGCATTGAGCATCATCCCAACTTTCGTGCAGTT |
| | | AAACACGTCCCATTTGACCAGTTTATACAGTTGGTTGCCCATGCTGGCTGTATGATTGGGAACAG |
| | | CAGCTGTGGGGTTCGAGAAGTTGGAGCTTTTGGAACACCTGTGATCAACCTGGGAACACGTCAGA |
| | | TTGGAAGAGAAACAGGGGAGAATGTTCTTCATGTCCGGGATGCTGACACCCAAGACAAAATATTG |
| | | CAAGCACTGCACCTTCAGTTTGGTAAACAGTACCCTTGTTCAAAGATATATGGGGATGGAAATGC |
| | | TGTTCCAAGGATTTTGAAGTTTCTCAAATCTATCGATCTTCAAGAGCCACTGCAAAAGAAATTCT |
| | | GCTTTCCTCCTGTGAAGGAGAATATCTCTCAAGATATTGACCATATTCTTGAAACTCTAAGTGCC |
| | | TTGGCCGTTGATCTTGGCGGGACGAACCTCCGAGTTGCAATAGTCAGCATGAAGGGTGAAATAGT |
| | | TAAGAAGTATACTCAGTTCAATCCTAAAACCTATGAAGAGAGGATTAATTTAATCCTACAGATGT |
| | | GTGTGGAAGCTGCAGCAGAAGCTGTAAAACTGAACTGCAGAATTTTGGGAGTAGGCATTTCCACA |
| | | GGTGGCCGTGTAAATCCTCGGGAAGGAATTGTGCTGCATTCAACCAAACTGATCCAAGAGTGGAA |
| | | CTCTGTGGACCTTAGGACCCCCCTTTCTGACACTTTGCATCTCCCTGTGTGGGTAGACAATGATG |
| | | GCAACTGTGCTGCCCTGGCGGAAAGGAAATTTGGCCAAGGAAAGGGACTGGAAAACTTTGTTACA |
| | | CTTATCACAGGCACAGGAATCGGTGGTGGAATTATCCATCAGCATGAATTGATCCACGGAAGCTC |
| | | CTTCTGTGCTGCAGAACTGGGCCACCTTGTTGTGTCTCTGGATGGGCCTGATTGTTCCTGTGGAA |
| | | GCCATGGGTGCATTGAAGCATACGCCTCTGGAATGGCCTTGCAGAGGGAGGCAAAAAAGCTCCAT |
| | | GATGAGGACCTGCTCTTGGTGGAAGGGATGTCAGTGCCAAAAGATGAGGCTGTGGGTGCGCTCCA |
| | | TCTCATCCAAGCTGCGAAACTTGGCAATGCGAAGGCCCAGAGCATCCTAAGAACAGCTGGAACAG |
| | | CTTTGGGTCTTGGGGTTGTGAACATCCTCCATACCATGAATCCCTCCCTTGTGATCCTCTCCGGA |
| | | GTCCTGGCCAGTCACTATATCCACATTGTCAAAGACGTCATTCGCCAGCAGGCCTTGTCCTCCGT |
| | | GCAGGACGTGGATGTGGTGGTTTCGGATTTGGTTGACCCCGCCCTGCTGGGTGCTGCCAGCATGG |
| | | TTCTGGACTACACAACACGCAGGATCTACTAGACCTCCAGGAACAGACATGGACCTTCTCTCCAG |
| | | AGCTCCTGAGTGGAATCAAGTTCTTGTCTTTAGGATGACCGTTTCTTAACAATCAAATCTGGTAT |
| | | TGAACTGCAGGTGACTTTGGCAGAGAAATGTTTTCACTTTTGGTCTCCTCTTCCAGAGTCACCTT |
| | | TCCCCACTCCTATTTTTGgTTTCTTCCCCATTAGGGATTATGCTGAATTTGTATGGTTTATAGTT |
| | | GTTAGAGTTTGAGGTGTTAATTCTAATTATCTATTTCAAATTTAGCAGGAAAAAAGAGAACATCA |
| | | CCTTGTAAAACTGAAGATTGTGACCAGTCAGAATAATGTCCTGCTGGGTGCTGCCAGCACGGAGA |
| | | TATGTGCATCTCCGTGCTGGCAGCACCCAGCAGGCATTATGGTGACAGCTGCCTCGGGAAGCCAA |
| | | GTTGGGCTTTAAAGTGCAGGGCCTGCTGATGTTGAGTGCTTTTTGTTCCATGCTGTGTATTGCCA |
| | | TCATCGAGTGAAGTAGATTAGCATCTCCGTGCTGGCAGCACCCAGCAGGCATAAGAAGTTATGTA |
| | | TTCATCCAATAATTCAAGCCAAGCAAGTATATAGGTGTTTTAATAGTTTTTGTTTTGGGGATTGT |
| | | GACCAGAAGATTTTGAAAATTAAATATTACTGAAGATTTCGACTTCCACTGTTAAATGTACAAGA |
| | | TACATGAAATATTAAAGAAAATGTGTAACTTTTTGTGTAAATACATCTTGTttttccctctgcca |
| | | aaaattatggggacatcatgaagccccttgagcatctgacttctggctaataaaggaaatttatt |
| | | ttcattgcaatagtgtgttggaattttttgtgtctctcactcggaaggacatatgggagggcaaa |
| | | tcatttaaaacatcagaatgagtatttggtttagagtttggcaacatatgcccattcttccgctt |
| | | cctcgctcactgactcgctgcgctcggtcgttcggctgcggcgagcggtatcagctcactcaaag |
| | | gcggtaatacggttatccacagaatcaggggataacgcaggaaagaacatgtgagcaaaaggcca |
| | | gcaaaaggccaggaaccgtaaaaaggccgcgttgctggcgtttttccataggctccgcccccctg |
| | | acgagcatcacaaaaatcgacgctcaagtcagaggtggcgaaacccgacaggactataaagatac |
| | | caggcgtttccccctggaagctccctcgtgcgctctcctgttccgaccctgccgcttaccggata |
| | | cctgtccgcctttctcccttcgggaagcgtggcgctttctcatagctcacgctgtaggtatctca |
| | | gttcggtgtaggtcgttcgctccaagctgggctgtgtgcacgaaccccccgttcagcccgaccgc |
| | | tgcgccttatccggtaactatcgtcttgagtccaacccggtaagacacgacttatcgccactggc |
| | | agcagccactggtaacaggattagcagagcgaggtatgtaggcggtgctacagagttcttgaagt |
| | | ggtggcctaactacggctacactagaagaacagtatttggtatctgcgctctgctgaagccagtt |
| | | accttcggaaaaagagttggtagctcttgatccggcaaacaaaccaccgctggtagcggtggttt |
| | | ttttgtttgcaagcagcagattacgcgcagaaaaaaaggatctcaagaagatcctttgatctttt |
| | | ctacggggtctgacgctcagtggaacgaaaactcacgttaagggattttggtcatgagattatca |
| | | aaaaggatcttcacctagatccttttaaattaaaaatgaagttttaaatcaatctaaagtatata |
| | | tgagtaaacttggtctgacagttaccaatgcttaatcagtgaggcacctatctcagcgatctgtc |
| | | tatttcgttcatccatagttgcctgactcggggggggggggcgctgaggtctgcctcgtgaagaa |
| | | ggtgttgctgactcataccaggcctgaatcgccccatcatccagccagaaagtgagggagccacg |
| | | gttgatgagagctttgttgtaggtggaccagttggtgattttgaacttttgctttgccacggaac |
| | | ggtctgcgttgtcgggaagatgcgtgatctgatccttcaactcagcaaaagttcgatttattcaa |
| | | caaagccgccgtcccgtcaagtcagcgtaatgctctgccagtgttacaaccaattaaccaattct |
| | | gattagaaaaactcatcgagcatcaaatgaaactgcaatttattcatatcaggattatcaatacc |
| | | atatttttgaaaaagccgtttctgtaatgaaggagaaaactcaccgaggcagttccataggatgg |
| | | caagatcctggtatcggtctgcgattccgactcgtccaacatcaatacaacctattaatttcccc |
| | | tcgtcaaaaataaggttatcaagtgagaaatcaccatgagtgacgactgaatccggtgagaatgg |
| | | caaaagcttatgcatttctttccagacttgttcaacaggccagccattacgctcgtcatcaaaat |
| | | cactcgcatcaaccaaaccgttattcattcgtgattgcgcctgagcgagacgaaatacgcgatcg |
| | | ctgttaaaaggacaattacaaacaggaatcgaatgcaaccggcgcaggaacactgccagcgcatc |
| | | aacaatattttcacctgaatcaggatattcttctaatacctggaatgctgttttcccggggatcg |
| | | cagtggtgagtaaccatgcatcatcaggagtacggataaaatgcttgatggtcggaagaggcata |
| | | aattccgtcagccagtttagtctgaccatctcatctgtaacatcattggcaacgctacctttgcc |
| | | atgtttcagaaacaactctggcgcatcgggcttcccatacaatcgatagattgtcgcacctgatt |
| | | gcccgacattatcgcgagcccatttatacccatataaatcagcatccatgttggaatttaatcgc |
| | | ggcctcgagcaagacgtttcccgttgaatatggctcataacaccccttgtattactgtttatgta |
| | | agcagacagttttattgttcatgatgatatatttttatcttgtgcaatgtaacatcagagatttt |
| | | gagacacaacgtggctttccccccccccccattattgaagcatttatcagggttattgtctcatg |
| | | agcggatacatatttgaatgtatttagaaaaataaacaaataggggttccgcgcacatttccccg |
| | | aaaagtgccacctgacgtctaagaaaccattattatcatgacattaacctataaaaataggcgta |
| | | tcacgaggccctttcgtctcgcgcgtttcggtgatgacggtgaaaacctctgacacatgcagctc |
| | | ccggagacggtcacagcttgtctgtaagcggatgccgggagcagacaagcccgtcagggcgcgtc |
| | | agcgggtgttggcgggtgtcggggctggcttaactatgcggcatcagagcagattgtactgagag |
| | | tgcaccatatgcggtgtgaaataccgcacagatgcgtaaggagaaaataccgcatcagattggct |
| | | at |
| 16 | mRNA transcript | ATGGAGAAGAATGGAAATAACCGAAAGCTGCGGGTTTGTGTTGCTACTTGTAACCGTGCAGATTA |
| | encoding mutant | TTCTAAACTTGCCCCGATCATGTTTGGCATTAAAACCGAACCTGAGTTCTTTGAACTTGATGTTG |
| | hGNE1 with D176V, | TGGTACTTGGCTCTCACCTGATAGATGACTATGGAAATACATATCGAATGATTGAACAAGATGAC |
| | V572L, and M712T | TTTGACATTAACACCAGGCTACACACAATTGTGAGGGGAGAAGATGAGGCAGCCATGGTGGAGTC |
| | | AGTAGGCCTGGCCCTAGTGAAGCTGCCAGATGTCCTTAATCGCCTGAAGCCTGATATCATGATTG |
| | | TTCATGGAGACAGGTTTGATGCCCTGGCTCTGGCCACATCTGCTGCCTTGATGAACATCCGAATC |
| | | CTTCACATTGAAGGTGGGGAAGTCAGTGGGACCATTGATGACTCTATCAGACATGCCATAACAAA |
| | | ACTGGCTCATTATCATGTGTGCTGCACCCGCAGTGCAGAGCAGCACCTGATATCCATGTGTGAGG |
| | | ACCATGTTCGCATCCTTTTGGCAGGCTGCCCTTCCTATGACAAACTTCTCTCAGCCAAGAACAAA |
| | | GACTACATGAGCATCATTCGCATGTGGCTAGGTGATGATGTAAAATCTAAAGATTACATTGTTGC |
| | | ACTACAGCACCCTGTGACCACTGACATTAAGCATTCCATAAAAATGTTTGAATTAACATTGGATG |
| | | CACTTATCTCATTTAACAAGCGGACCCTAGTCCTGTTTCCAAATATTGACGCAGGGAGCAAAGAG |
| | | ATGGTTCGAGTGATGCGGAAGAAGGGCATTGAGCATCATCCCAACTTTCGTGCAGTTAAACACGT |
| | | CCCATTTGACCAGTTTATACAGTTGGTTGCCCATGCTGGCTGTATGATTGGGAACAGCAGCTGTG |
| | | GGGTTCGAGAAGTTGGAGCTTTTGGAACACCTGTGATCAACCTGGGAACACGTCAGATTGGAAGA |
| | | GAAACAGGGGAGAATGTTCTTCATGTCCGGGATGCTGACACCCAAGACAAAATATTGCAAGCACT |
| | | GCACCTTCAGTTTGGTAAACAGTACCCTTGTTCAAAGATATATGGGGATGGAAATGCTGTTCCAA |
| | | GGATTTTGAAGTTTCTCAAATCTATCGATCTTCAAGAGCCACTGCAAAAGAAATTCTGCTTTCCT |
| | | CCTGTGAAGGAGAATATCTCTCAAGATATTGACCATATTCTTGAAACTCTAAGTGCCTTGGCCGT |
| | | TGATCTTGGCGGGACGAACCTCCGAGTTGCAATAGTCAGCATGAAGGGTGAAATAGTTAAGAAGT |
| | | ATACTCAGTTCAATCCTAAAACCTATGAAGAGAGGATTAATTTAATCCTACAGATGTGTGTGGAA |
| | | GCTGCAGCAGAAGCTGTAAAACTGAACTGCAGAATTTTGGGAGTAGGCATTTCCACAGGTGGCCG |
| | | TGTAAATCCTCGGGAAGGAATTGTGCTGCATTCAACCAAACTGATCCAAGAGTGGAACTCTGTGG |
| | | ACCTTAGGACCCCCCTTTCTGACACTTTGCATCTCCCTGTGTGGGTAGACAATGATGGCAACTGT |
| | | GCTGCCCTGGCGGAAAGGAAATTTGGCCAAGGAAAGGGACTGGAAAACTTTGTTACACTTATCAC |
| | | AGGCACAGGAATCGGTGGTGGAATTATCCATCAGCATGAATTGATCCACGGAAGCTCCTTCTGTG |
| | | CTGCAGAACTGGGCCACCTTGTTCTGTCTCTGGATGGGCCTGATTGTTCCTGTGGAAGCCATGGG |
| | | TGCATTGAAGCATACGCCTCTGGAATGGCCTTGCAGAGGGAGGCAAAAAAGCTCCATGATGAGGA |
| | | CCTGCTCTTGGTGGAAGGGATGTCAGTGCCAAAAGATGAGGCTGTGGGTGCGCTCCATCTCATCC |
| | | AAGCTGCGAAACTTGGCAATGCGAAGGCCCAGAGCATCCTAAGAACAGCTGGAACAGCTTTGGGT |
| | | CTTGGGGTTGTGAACATCCTCCATACCATGAATCCCTCCCTTGTGATCCTCTCCGGAGTCCTGGC |
| | | CAGTCACTATATCCACATTGTCAAAGACGTCATTCGCCAGCAGGCCTTGTCCTCCGTGCAGGACG |
| | | TGGATGTGGTGGTTTCGGATTTGGTTGACCCCGCCCTGCTGGGTGCTGCCAGCACGGTTCTGGAC |
| | | TACACAACACGCAGGATCTACTAG |
| 17 | mRNA transcript | ATGGAGAAGAATGGAAATAACCGAAAGCTGCGGGTTTGTGTTGCTACTTGTAACCGTGCAGATTA |
| | encoding mutant | TTCTAAACTTGCCCCGATCATGTTTGGCATTAAAACCGAACCTGAGTTCTTTGAACTTGATGTTG |
| | hGNE1 with D176V | TGGTACTTGGCTCTCACCTGATAGATGACTATGGAAATACATATCGAATGATTGAACAAGATGAC |
| | | TTTGACATTAACACCAGGCTACACACAATTGTGAGGGGAGAAGATGAGGCAGCCATGGTGGAGTC |
| | | AGTAGGCCTGGCCCTAGTGAAGCTGCCAGATGTCCTTAATCGCCTGAAGCCTGATATCATGATTG |
| | | TTCATGGAGACAGGTTTGATGCCCTGGCTCTGGCCACATCTGCTGCCTTGATGAACATCCGAATC |
| | | CTTCACATTGAAGGTGGGGAAGTCAGTGGGACCATTGATGACTCTATCAGACATGCCATAACAAA |
| | | ACTGGCTCATTATCATGTGTGCTGCACCCGCAGTGCAGAGCAGCACCTGATATCCATGTGTGAGG |
| | | ACCATGTTCGCATCCTTTTGGCAGGCTGCCCTTCCTATGACAAACTTCTCTCAGCCAAGAACAAA |
| | | GACTACATGAGCATCATTCGCATGTGGCTAGGTGATGATGTAAAATCTAAAGATTACATTGTTGC |
| | | ACTACAGCACCCTGTGACCACTGACATTAAGCATTCCATAAAAATGTTTGAATTAACATTGGATG |
| | | CACTTATCTCATTTAACAAGCGGACCCTAGTCCTGTTTCCAAATATTGACGCAGGGAGCAAAGAG |
| | | ATGGTTCGAGTGATGCGGAAGAAGGGCATTGAGCATCATCCCAACTTTCGTGCAGTTAAACACGT |
| | | CCCATTTGACCAGTTTATACAGTTGGTTGCCCATGCTGGCTGTATGATTGGGAACAGCAGCTGTG |
| | | GGGTTCGAGAAGTTGGAGCTTTTGGAACACCTGTGATCAACCTGGGAACACGTCAGATTGGAAGA |
| | | GAAACAGGGGAGAATGTTCTTCATGTCCGGGATGCTGACACCCAAGACAAAATATTGCAAGCACT |
| | | GCACCTTCAGTTTGGTAAACAGTACCCTTGTTCAAAGATATATGGGGATGGAAATGCTGTTCCAA |
| | | GGATTTTGAAGTTTCTCAAATCTATCGATCTTCAAGAGCCACTGCAAAAGAAATTCTGCTTTCCT |
| | | CCTGTGAAGGAGAATATCTCTCAAGATATTGACCATATTCTTGAAACTCTAAGTGCCTTGGCCGT |
| | | TGATCTTGGCGGGACGAACCTCCGAGTTGCAATAGTCAGCATGAAGGGTGAAATAGTTAAGAAGT |
| | | ATACTCAGTTCAATCCTAAAACCTATGAAGAGAGGATTAATTTAATCCTACAGATGTGTGTGGAA |
| | | GCTGCAGCAGAAGCTGTAAAACTGAACTGCAGAATTTTGGGAGTAGGCATTTCCACAGGTGGCCG |
| | | TGTAAATCCTCGGGAAGGAATTGTGCTGCATTCAACCAAACTGATCCAAGAGTGGAACTCTGTGG |
| | | ACCTTAGGACCCCCCTTTCTGACACTTTGCATCTCCCTGTGTGGGTAGACAATGATGGCAACTGT |
| | | GCTGCCCTGGCGGAAAGGAAATTTGGCCAAGGAAAGGGACTGGAAAACTTTGTTACACTTATCAC |
| | | AGGCACAGGAATCGGTGGTGGAATTATCCATCAGCATGAATTGATCCACGGAAGCTCCTTCTGTG |
| | | CTGCAGAACTGGGCCACCTTGTTGTGTCTCTGGATGGGCCTGATTGTTCCTGTGGAAGCCATGGG |
| | | TGCATTGAAGCATACGCCTCTGGAATGGCCTTGCAGAGGGAGGCAAAAAAGCTCCATGATGAGGA |
| | | CCTGCTCTTGGTGGAAGGGATGTCAGTGCCAAAAGATGAGGCTGTGGGTGCGCTCCATCTCATCC |
| | | AAGCTGCGAAACTTGGCAATGCGAAGGCCCAGAGCATCCTAAGAACAGCTGGAACAGCTTTGGGT |
| | | CTTGGGGTTGTGAACATCCTCCATACCATGAATCCCTCCCTTGTGATCCTCTCCGGAGTCCTGGC |
| | | CAGTCACTATATCCACATTGTCAAAGACGTCATTCGCCAGCAGGCCTTGTCCTCCGTGCAGGACG |
| | | TGGATGTGGTGGTTTCGGATTTGGTTGACCCCGCCCTGCTGGGTGCTGCCAGCATGGTTCTGGAC |
| | | TACACAACACGCAGGATCTACTAG |
| 18 | mRNA transcript | ATGGAGAAGAATGGAAATAACCGAAAGCTGCGGGTTTGTGTTGCTACTTGTAACCGTGCAGATTA |
| | encoding mutant | TTCTAAACTTGCCCCGATCATGTTTGGCATTAAAACCGAACCTGAGTTCTTTGAACTTGATGTTG |
| | hGNE1 with V572L | TGGTACTTGGCTCTCACCTGATAGATGACTATGGAAATACATATCGAATGATTGAACAAGATGAC |
| | | TTTGACATTAACACCAGGCTACACACAATTGTGAGGGGAGAAGATGAGGCAGCCATGGTGGAGTC |
| | | AGTAGGCCTGGCCCTAGTGAAGCTGCCAGATGTCCTTAATCGCCTGAAGCCTGATATCATGATTG |
| | | TTCATGGAGACAGGTTTGATGCCCTGGCTCTGGCCACATCTGCTGCCTTGATGAACATCCGAATC |
| | | CTTCACATTGAAGGTGGGGAAGTCAGTGGGACCATTGATGACTCTATCAGACATGCCATAACAAA |
| | | ACTGGCTCATTATCATGTGTGCTGCACCCGCAGTGCAGAGCAGCACCTGATATCCATGTGTGAGG |
| | | ACCATGATCGCATCCTTTTGGCAGGCTGCCCTTCCTATGACAAACTTCTCTCAGCCAAGAACAAA |
| | | GACTACATGAGCATCATTCGCATGTGGCTAGGTGATGATGTAAAATCTAAAGATTACATTGTTGC |
| | | ACTACAGCACCCTGTGACCACTGACATTAAGCATTCCATAAAAATGTTTGAATTAACATTGGATG |
| | | CACTTATCTCATTTAACAAGCGGACCCTAGTCCTGTTTCCAAATATTGACGCAGGGAGCAAAGAG |
| | | ATGGTTCGAGTGATGCGGAAGAAGGGCATTGAGCATCATCCCAACTTTCGTGCAGTTAAACACGT |
| | | CCCATTTGACCAGTTTATACAGTTGGTTGCCCATGCTGGCTGTATGATTGGGAACAGCAGCTGTG |
| | | GGGTTCGAGAAGTTGGAGCTTTTGGAACACCTGTGATCAACCTGGGAACACGTCAGATTGGAAGA |
| | | GAAACAGGGGAGAATGTTCTTCATGTCCGGGATGCTGACACCCAAGACAAAATATTGCAAGCACT |
| | | GCACCTTCAGTTTGGTAAACAGTACCCTTGTTCAAAGATATATGGGGATGGAAATGCTGTTCCAA |
| | | GGATTTTGAAGTTTCTCAAATCTATCGATCTTCAAGAGCCACTGCAAAAGAAATTCTGCTTTCCT |
| | | CCTGTGAAGGAGAATATCTCTCAAGATATTGACCATATTCTTGAAACTCTAAGTGCCTTGGCCGT |
| | | TGATCTTGGCGGGACGAACCTCCGAGTTGCAATAGTCAGCATGAAGGGTGAAATAGTTAAGAAGT |
| | | ATACTCAGTTCAATCCTAAAACCTATGAAGAGAGGATTAATTTAATCCTACAGATGTGTGTGGAA |
| | | GCTGCAGCAGAAGCTGTAAAACTGAACTGCAGAATTTTGGGAGTAGGCATTTCCACAGGTGGCCG |
| | | TGTAAATCCTCGGGAAGGAATTGTGCTGCATTCAACCAAACTGATCCAAGAGTGGAACTCTGTGG |
| | | ACCTTAGGACCCCCCTTTCTGACACTTTGCATCTCCCTGTGTGGGTAGACAATGATGGCAACTGT |
| | | GCTGCCCTGGCGGAAAGGAAATTTGGCCAAGGAAAGGGACTGGAAAACTTTGTTACACTTATCAC |
| | | AGGCACAGGAATCGGTGGTGGAATTATCCATCAGCATGAATTGATCCACGGAAGCTCCTTCTGTG |
| | | CTGCAGAACTGGGCCACCTTGTTCTGTCTCTGGATGGGCCTGATTGTTCCTGTGGAAGCCATGGG |
| | | TGCATTGAAGCATACGCCTCTGGAATGGCCTTGCAGAGGGAGGCAAAAAAGCTCCATGATGAGGA |
| | | CCTGCTCTTGGTGGAAGGGATGTCAGTGCCAAAAGATGAGGCTGTGGGTGCGCTCCATCTCATCC |
| | | AAGCTGCGAAACTTGGCAATGCGAAGGCCCAGAGCATCCTAAGAACAGCTGGAACAGCTTTGGGT |
| | | CTTGGGGTTGTGAACATCCTCCATACCATGAATCCCTCCCTTGTGATCCTCTCCGGAGTCCTGGC |
| | | CAGTCACTATATCCACATTGTCAAAGACGTCATTCGCCAGCAGGCCTTGTCCTCCGTGCAGGACG |
| | | TGGATGTGGTGGTTTCGGATTTGGTTGACCCCGCCCTGCTGGGTGCTGCCAGCATGGTTCTGGAC |
| | | TACACAACACGCAGGATCTACTAG |
| 19 | mRNA transcript | ATGGAGAAGAATGGAAATAACCGAAAGCTGCGGGTTTGTGTTGCTACTTGTAACCGTGCAGATTA |
| | encoding mutant | TTCTAAACTTGCCCCGATCATGTTTGGCATTAAAACCGAACCTGAGTTCTTTGAACTTGATGTTG |
| | hGNE1 M712T | TGGTACTTGGCTCTCACCTGATAGATGACTATGGAAATACATATCGAATGATTGAACAAGATGAC |
| | | TTTGACATTAACACCAGGCTACACACAATTGTGAGGGGAGAAGATGAGGCAGCCATGGTGGAGTC |
| | | AGTAGGCCTGGCCCTAGTGAAGCTGCCAGATGTCCTTAATCGCCTGAAGCCTGATATCATGATTG |
| | | TTCATGGAGACAGGTTTGATGCCCTGGCTCTGGCCACATCTGCTGCCTTGATGAACATCCGAATC |
| | | CTTCACATTGAAGGTGGGGAAGTCAGTGGGACCATTGATGACTCTATCAGACATGCCATAACAAA |
| | | ACTGGCTCATTATCATGTGTGCTGCACCCGCAGTGCAGAGCAGCACCTGATATCCATGTGTGAGG |
| | | ACCATGATCGCATCCTTTTGGCAGGCTGCCCTTCCTATGACAAACTTCTCTCAGCCAAGAACAAA |
| | | GACTACATGAGCATCATTCGCATGTGGCTAGGTGATGATGTAAAATCTAAAGATTACATTGTTGC |
| | | ACTACAGCACCCTGTGACCACTGACATTAAGCATTCCATAAAAATGTTTGAATTAACATTGGATG |
| | | CACTTATCTCATTTAACAAGCGGACCCTAGTCCTGTTTCCAAATATTGACGCAGGGAGCAAAGAG |
| | | ATGGTTCGAGTGATGCGGAAGAAGGGCATTGAGCATCATCCCAACTTTCGTGCAGTTAAACACGT |
| | | CCCATTTGACCAGTTTATACAGTTGGTTGCCCATGCTGGCTGTATGATTGGGAACAGCAGCTGTG |
| | | GGGTTCGAGAAGTTGGAGCTTTTGGAACACCTGTGATCAACCTGGGAACACGTCAGATTGGAAGA |
| | | GAAACAGGGGAGAATGTTCTTCATGTCCGGGATGCTGACACCCAAGACAAAATATTGCAAGCACT |
| | | GCACCTTCAGTTTGGTAAACAGTACCCTTGTTCAAAGATATATGGGGATGGAAATGCTGTTCCAA |
| | | GGATTTTGAAGTTTCTCAAATCTATCGATCTTCAAGAGCCACTGCAAAAGAAATTCTGCTTTCCT |
| | | CCTGTGAAGGAGAATATCTCTCAAGATATTGACCATATTCTTGAAACTCTAAGTGCCTTGGCCGT |
| | | TGATCTTGGCGGGACGAACCTCCGAGTTGCAATAGTCAGCATGAAGGGTGAAATAGTTAAGAAGT |
| | | ATACTCAGTTCAATCCTAAAACCTATGAAGAGAGGATTAATTTAATCCTACAGATGTGTGTGGAA |
| | | GCTGCAGCAGAAGCTGTAAAACTGAACTGCAGAATTTTGGGAGTAGGCATTTCCACAGGTGGCCG |
| | | TGTAAATCCTCGGGAAGGAATTGTGCTGCATTCAACCAAACTGATCCAAGAGTGGAACTCTGTGG |
| | | ACCTTAGGACCCCCCTTTCTGACACTTTGCATCTCCCTGTGTGGGTAGACAATGATGGCAACTGT |
| | | GCTGCCCTGGCGGAAAGGAAATTTGGCCAAGGAAAGGGACTGGAAAACTTTGTTACACTTATCAC |
| | | AGGCACAGGAATCGGTGGTGGAATTATCCATCAGCATGAATTGATCCACGGAAGCTCCTTCTGTG |
| | | CTGCAGAACTGGGCCACCTTGTTGTGTCTCTGGATGGGCCTGATTGTTCCTGTGGAAGCCATGGG |
| | | TGCATTGAAGCATACGCCTCTGGAATGGCCTTGCAGAGGGAGGCAAAAAAGCTCCATGATGAGGA |
| | | CCTGCTCTTGGTGGAAGGGATGTCAGTGCCAAAAGATGAGGCTGTGGGTGCGCTCCATCTCATCC |
| | | AAGCTGCGAAACTTGGCAATGCGAAGGCCCAGAGCATCCTAAGAACAGCTGGAACAGCTTTGGGT |
| | | CTTGGGGTTGTGAACATCCTCCATACCATGAATCCCTCCCTTGTGATCCTCTCCGGAGTCCTGGC |
| | | CAGTCACTATATCCACATTGTCAAAGACGTCATTCGCCAGCAGGCCTTGTCCTCCGTGCAGGACG |
| | | TGGATGTGGTGGTTTCGGATTTGGTTGACCCCGCCCTGCTGGGTGCTGCCAGCACGGTTCTGGAC |
| | | TACACAACACGCAGGATCTACTAG |
| 20 | Vector expressing | tggccattgcatacgttgtatccatatcataatatgtacatttatattggctcatgtccaacatt |
| | hGNE2 and | accgccatgttgacattgattattgactagttattaatagtaatcaattacggggtcattagttc |
| | knockdown M743T | atagcccatatatggagttccgcgttacataacttacggtaaatggcccgcctggctgaccgccc |
| | hGNE2 mutation in | aacgacccccgcccattgacgtcaataatgacgtatgttcccatagtaacgccaatagggacttt |
| | pUMVC3 backbone (bi- | ccattgacgtcaatgggtggagtatttacggtaaactgcccacttggcagtacatcaagtgtatc |
| | shRNA is in front | atatgccaagtacgccccctattgacgtcaatgacggtaaatggcccgcctggcattatgcccag |
| | of the hGNE2 mRNA | tacatgaccttatgggactttcctacttggcagtacatctacgtattagtcatcgctattaccat |
| | unit; sequence of | ggtgatgcggttttggcagtacatcaatgggcgtggatagcggtttgactcacggggatttccaa |
| | bi-shRNA is in bold) | gtctccaccccattgacgtcaatgggagtttgttttggcaccaaaatcaacgggactttccaaaa |
| | | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgactttcttccccattagggattatgctgaatttgtatggtttatagttgtta |
| | | gagtttgaggtgttaattctaattatctatttcaaatttagcaggaaaaaagagaacatcacctt |
| | | gtaaaactgaagattgtgaccagtcagaataatgtcctgctgggtgctgccagcacggagatatg |
| | | tgcatctccgtgctggcagcacccagcaggcattatggtgacagctgcctcgggaagccaagttg |
| | | ggctttaaagtgcagggcctgctgatgttgagtgctttttgttccatactgtgtatagccatcat |
| | | cgagtgaagtagattagcatctccgtgctggcagcacccagcaggcataagaagttatgtattca |
| | | tccaataattcaagccaagcaagtatataggtgttttaatagtttttgttttggggattgtgacc |
| | | agaagattttgaaaattaaatattactgaagatttcgacttccactgttaaatgtacaagataca |
| | | tgaaatattaaagaaaatgtgtaactttttgtgtaaatacatcttgttcaccgtcgtcgacggta |
| | | tcgataagcttgatatcgaattcgcccttatggagaagaatggaaataaccgaaagctgcgggtt |
| | | tgtgttgctacttgtaaccgtgcagattattctaaacttgccccgatcatgtttggcattaaaac |
| | | cgaacctgagttctttgaacttgatgttgtggtacttggctctcacctgatagatgactatggaa |
| | | atacatatcgaatgattgaacaagatgactttgacattaacaccaggctacacacaattgtgagg |
| | | ggagaagatgaggcagccatggtggagtcagtaggcctggccctagtgaagctgccagatgtcct |
| | | taatcgcctgaagcctgatatcatgattgttcatggagacaggtttgatgccctggctctggcca |
| | | catctgctgccttgatgaacatccgaatccttcacattgaaggtggggaagtcagtgggaccatt |
| | | gatgactctatcagacatgccataacaaaactggctcattatcatgtgtgctgcacccgcagtgc |
| | | agagcagcacctgatatccatgtgtgaggaccatgatcgcatccttttggcaggctgcccttcct |
| | | atgacaaacttctctcagccaagaacaaagactacatgagcatcattcgcatgtggctaggtgat |
| | | gatgtaaaatctaaagattacattgttgcactacagcaccctgtgaccactgacattaagcattc |
| | | cataaaaatgtttgaattaacattggatgcacttatctcatttaacaagcggaccctagtcctgt |
| | | ttccaaatattgacgcagggagcaaagagatggttcgagtgatgcggaagaagggcattgagcat |
| | | catcccaactttcgtgcagttaaacacgtcccatttgaccagtttatacagttggttgcccatgc |
| | | tggctgtatgattgggaacagcagctgtggggttcgagaagttggagcttttggaacacctgtga |
| | | tcaacctgggaacacgtcagattggaagagaaacaggggagaatgttcttcatgtccgggatgct |
| | | gacacccaagacaaaatattgcaagcactgcaccttcagtttggtaaacagtacccttgttcaaa |
| | | gatatatggggatggaaatgctgttccaaggattttgaagtttctcaaatctatcgatcttcaag |
| | | agccactgcaaaagaaattctgctttcctcctgtgaaggagaatatctctcaagatattgaccat |
| | | attcttgaaactctaagtgccttggccgttgatcttggcgggacgaacctccgagttgcaatagt |
| | | cagcatgaagggtgaaatagttaagaagtatactcagttcaatcctaaaacctatgaagagagga |
| | | ttaatttaatcctacagatgtgtgtggaagctgcagcagaagctgtaaaactgaactgcagaatt |
| | | ttgggagtaggcatttccacaggtggccgtgtaaatcctcgggaaggaattgtgctgcattcaac |
| | | caaactgatccaagagtggaactctgtggaccttaggacccccctttctgacactttgcatctcc |
| | | ctgtgtgggtagacaatgatggcaactgtgctgccctggcggaaaggaaatttggccaaggaaag |
| | | ggactggaaaactttgttacacttatcacaggcacaggaatcggtggtggaattatccatcagca |
| | | tgaattgatccacggaagctccttctgtgctgcagaactgggccaccttgttgtgtctctggatg |
| | | ggcctgattgttcctgtggaagccatgggtgcattgaagcatacgcctctggaatggccttgcag |
| | | agggaggcaaaaaagctccatgatgaggacctgctcttggtggaagggatgtcagtgccaaaaga |
| | | tgaggctgtgggtgcgctccatctcatccaagctgcgaaacttggcaatgcgaaggcccagagca |
| | | tcctaagaacagctggaacagctttgggtcttggggttgtgaacatcctccataccatgaatccc |
| | | tcccttgtgatcctctccggagtcctggccagtcactatatccacattgtcaaagacgtcattcg |
| | | ccagcaggccttgtcctccgtgcaggacgtggatgtggtggtttcggatttggttgaccccgccc |
| | | tgctgggtgctgccagcatggttctggactacacaacacgcaggatctactagaagggcgaattc |
| | | acgtgggcccggtaccgtatactctagagcggccgcggatccagatctttttccctctgccaaaa |
| | | attatggggacatcatgaagccccttgagcatctgacttctggctaataaaggaaatttattttc |
| | | attgcaatagtgtgttggaattttttgtgtctctcactcggaaggacatatgggagggcaaatca |
| | | tttaaaacatcagaatgagtatttggtttagagtttggcaacatatgcccattcttccgcttcct |
| | | cgctcactgactcgctgcgctcggtcgttcggctgcggcgagcggtatcagctcactcaaaggcg |
| | | gtaatacggttatccacagaatcaggggataacgcaggaaagaacatgtgagcaaaaggccagca |
| | | aaaggccaggaaccgtaaaaaggccgcgttgctggcgtttttccataggctccgcccccctgacg |
| | | agcatcacaaaaatcgacgctcaagtcagaggtggcgaaacccgacaggactataaagataccag |
| | | gcgtttccccctggaagctccctcgtgcgctctcctgttccgaccctgccgcttaccggatacct |
| | | gtccgcctttctcccttcgggaagcgtggcgctttctcatagctcacgctgtaggtatctcagtt |
| | | cggtgtaggtcgttcgctccaagctgggctgtgtgcacgaaccccccgttcagcccgaccgctgc |
| | | gccttatccggtaactatcgtcttgagtccaacccggtaagacacgacttatcgccactggcagc |
| | | agccactggtaacaggattagcagagcgaggtatgtaggcggtgctacagagttcttgaagtggt |
| | | ggcctaactacggctacactagaagaacagtatttggtatctgcgctctgctgaagccagttacc |
| | | ttcggaaaaagagttggtagctcttgatccggcaaacaaaccaccgctggtagcggtggtttttt |
| | | tgtttgcaagcagcagattacgcgcagaaaaaaaggatctcaagaagatcctttgatcttttcta |
| | | cggggtctgacgctcagtggaacgaaaactcacgttaagggattttggtcatgagattatcaaaa |
| | | aggatcttcacctagatccttttaaattaaaaatgaagttttaaatcaatctaaagtatatatga |
| | | gtaaacttggtctgacagttaccaatgcttaatcagtgaggcacctatctcagcgatctgtctat |
| | | ttcgttcatccatagttgcctgactcggggggggggggcgctgaggtctgcctcgtgaagaaggt |
| | | gttgctgactcataccaggcctgaatcgccccatcatccagccagaaagtgagggagccacggtt |
| | | gatgagagctttgttgtaggtggaccagttggtgattttgaacttttgctttgccacggaacggt |
| | | ctgcgttgtcgggaagatgcgtgatctgatccttcaactcagcaaaagttcgatttattcaacaa |
| | | agccgccgtcccgtcaagtcagcgtaatgctctgccagtgttacaaccaattaaccaattctgat |
| | | tagaaaaactcatcgagcatcaaatgaaactgcaatttattcatatcaggattatcaataccata |
| | | tttttgaaaaagccgtttctgtaatgaaggagaaaactcaccgaggcagttccataggatggcaa |
| | | gatcctggtatcggtctgcgattccgactcgtccaacatcaatacaacctattaatttcccctcg |
| | | tcaaaaataaggttatcaagtgagaaatcaccatgagtgacgactgaatccggtgagaatggcaa |
| | | aagcttatgcatttctttccagacttgttcaacaggccagccattacgctcgtcatcaaaatcac |
| | | tcgcatcaaccaaaccgttattcattcgtgattgcgcctgagcgagacgaaatacgcgatcgctg |
| | | ttaaaaggacaattacaaacaggaatcgaatgcaaccggcgcaggaacactgccagcgcatcaac |
| | | aatattttcacctgaatcaggatattcttctaatacctggaatgctgttttcccggggatcgcag |
| | | tggtgagtaaccatgcatcatcaggagtacggataaaatgcttgatggtcggaagaggcataaat |
| | | tccgtcagccagtttagtctgaccatctcatctgtaacatcattggcaacgctacctttgccatg |
| | | tttcagaaacaactctggcgcatcgggcttcccatacaatcgatagattgtcgcacctgattgcc |
| | | cgacattatcgcgagcccatttatacccatataaatcagcatccatgttggaatttaatcgcggc |
| | | ctcgagcaagacgtttcccgttgaatatggctcataacaccccttgtattactgtttatgtaagc |
| | | agacagttttattgttcatgatgatatatttttatcttgtgcaatgtaacatcagagattttgag |
| | | acacaacgtggctttccccccccccccattattgaagcatttatcagggttattgtctcatgagc |
| | | ggatacatatttgaatgtatttagaaaaataaacaaataggggttccgcgcacatttccccgaaa |
| | | agtgccacctgacgtctaagaaaccattattatcatgacattaacctataaaaataggcgtatca |
| | | cgaggccctttcgtctcgcgcgtttcggtgatgacggtgaaaacctctgacacatgcagctcccg |
| | | gagacggtcacagcttgtctgtaagcggatgccgggagcagacaagcccgtcagggcgcgtcagc |
| | | gggtgttggcgggtgtcggggctggcttaactatgcggcatcagagcagattgtactgagagtgc |
| | | accatatgcggtgtgaaataccgcacagatgcgtaaggagaaaataccgcatcagattggctat |
| 21 | Vector expressing | tggccattgcatacgttgtatccatatcataatatgtacatttatattggctcatgtccaacatt |
| | hGNE2 and | accgccatgttgacattgattattgactagttattaatagtaatcaattacggggtcattagttc |
| | knockdown M743T | atagcccatatatggagttccgcgttacataacttacggtaaatggcccgcctggctgaccgccc |
| | hGNE2 mutation in | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | (bi-shRNA is behind | atatgccaagtacgccccctattgacgtcaatgacggtaaatggcccgcctggcattatgcccag |
| | the hGNE2 mRNA | tacatgaccttatgggactttcctacttggcagtacatctacgtattagtcatcgctattaccat |
| | unit; sequence | ggtgatgcggttttggcagtacatcaatgggcgtggatagcggtttgactcacggggatttccaa |
| | of bi-shRNA | gtctccaccccattgacgtcaatgggagtttgttttggcaccaaaatcaacgggactttccaaaa |
| | is in bold) | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacgtaagtaccgcctatagactctataggcacacccctttggctcttatgca |
| | | tgctatactgtttttggcttggggcctatacacccccgcttccttatgctataggtgatggtata |
| | | gcttagcctataggtgtgggttattgaccattattgaccactccaacggtggagggcagtgtagt |
| | | ctgagcagtactcgttgctgccgcgcgcgccaccagacataatagctgacagactaacagactgt |
| | | tcctttccatgggtcttttctgcagtcaccgtcgtcgacggtatcgataagcttgatatcgaatt |
| | | cgcccttatggagaagaatggaaataaccgaaagctgcgggtttgtgttgctacttgtaaccgtg |
| | | cagattattctaaacttgccccgatcatgtttggcattaaaaccgaacctgagttctttgaactt |
| | | gatgttgtggtacttggctctcacctgatagatgactatggaaatacatatcgaatgattgaaca |
| | | agatgactttgacattaacaccaggctacacacaattgtgaggggagaagatgaggcagccatgg |
| | | tggagtcagtaggcctggccctagtgaagctgccagatgtccttaatcgcctgaagcctgatatc |
| | | atgattgttcatggagacaggtttgatgccctggctctggccacatctgctgccttgatgaacat |
| | | ccgaatccttcacattgaaggtggggaagtcagtgggaccattgatgactctatcagacatgcca |
| | | taacaaaactggctcattatcatgtgtgctgcacccgcagtgcagagcagcacctgatatccatg |
| | | tgtgaggaccatgatcgcatccttttggcaggctgcccttcctatgacaaacttctctcagccaa |
| | | gaacaaagactacatgagcatcattcgcatgtggctaggtgatgatgtaaaatctaaagattaca |
| | | ttgttgcactacagcaccctgtgaccactgacattaagcattccataaaaatgtttgaattaaca |
| | | ttggatgcacttatctcatttaacaagcggaccctagtcctgtttccaaatattgacgcagggag |
| | | caaagagatggttcgagtgatgcggaagaagggcattgagcatcatcccaactttcgtgcagtta |
| | | aacacgtcccatttgaccagtttatacagttggttgcccatgctggctgtatgattgggaacagc |
| | | agctgtggggttcgagaagttggagcttttggaacacctgtgatcaacctgggaacacgtcagat |
| | | tggaagagaaacaggggagaatgttcttcatgtccgggatgctgacacccaagacaaaatattgc |
| | | aagcactgcaccttcagtttggtaaacagtacccttgttcaaagatatatggggatggaaatgct |
| | | gttccaaggattttgaagtttctcaaatctatcgatcttcaagagccactgcaaaagaaattctg |
| | | ctttcctcctgtgaaggagaatatctctcaagatattgaccatattcttgaaactctaagtgcct |
| | | tggccgttgatcttggcgggacgaacctccgagttgcaatagtcagcatgaagggtgaaatagtt |
| | | aagaagtatactcagttcaatcctaaaacctatgaagagaggattaatttaatcctacagatgtg |
| | | tgtggaagctgcagcagaagctgtaaaactgaactgcagaattttgggagtaggcatttccacag |
| | | gtggccgtgtaaatcctcgggaaggaattgtgctgcattcaaccaaactgatccaagagtggaac |
| | | tctgtggaccttaggacccccctttctgacactttgcatctccctgtgtgggtagacaatgatgg |
| | | caactgtgctgccctggcggaaaggaaatttggccaaggaaagggactggaaaactttgttacac |
| | | ttatcacaggcacaggaatcggtggtggaattatccatcagcatgaattgatccacggaagctcc |
| | | ttctgtgctgcagaactgggccaccttgttgtgtctctggatgggcctgattgttcctgtggaag |
| | | ccatgggtgcattgaagcatacgcctctggaatggccttgcagagggaggcaaaaaagctccatg |
| | | atgaggacctgctcttggtggaagggatgtcagtgccaaaagatgaggctgtgggtgcgctccat |
| | | ctcatccaagctgcgaaacttggcaatgcgaaggcccagagcatcctaagaacagctggaacagc |
| | | tttgggtcttggggttgtgaacatcctccataccatgaatccctcccttgtgatcctctccggag |
| | | tcctggccagtcactatatccacattgtcaaagacgtcattcgccagcaggccttgtcctccgtg |
| | | caggacgtggatgtggtggtttcggatttggttgaccccgccctgctgggtgctgccagcatggt |
| | | tctggactacacaacacgcaggatctactagaagggcgaattcacgtgggcccggtaccgtatac |
| | | tctagagcggccgcggtttcttccccattagggattatgctgaatttgtatggtttatagttgt |
| | | tagagtttgaggtgttaattctaattatctatttcaaatttagcaggaaaaaagagaacatcacc |
| | | ttgtaaaactgaagattgtgaccagtcagaataatgtcctgctgggtgctgccagcacggagata |
| | | tgtgcatctccgtgctggcagcacccagcaggcattatggtgacagctgcctcgggaagccaagt |
| | | tgggctttaaagtgcagggcctgctgatgttgagtgctttttgttccatactgtgtatagccatc |
| | | atcgagtgaagtagattagcatctccgtgctggcagcacccagcaggcataagaagttatgtatt |
| | | catccaataattcaagccaagcaagtatataggtgttttaatagtttttgttttggggattgtga |
| | | ccagaagattttgaaaattaaatattactgaagatttcgacttccactgttaaatgtacaagata |
| | | catgaaatattaaagaaaatgtgtaactttttgtgtaaatacatcttgtatccagatctttttcc |
| | | ctctgccaaaaattatggggacatcatgaagccccttgagcatctgacttctggctaataaagga |
| | | aatttattttcattgcaatagtgtgttggaattttttgtgtctctcactcggaaggacatatggg |
| | | agggcaaatcatttaaaacatcagaatgagtatttggtttagagtttggcaacatatgcccattc |
| | | ttccgcttcctcgctcactgactcgctgcgctcggtcgttcggctgcggcgagcggtatcagctc |
| | | actcaaaggcggtaatacggttatccacagaatcaggggataacgcaggaaagaacatgtgagca |
| | | aaaggccagcaaaaggccaggaaccgtaaaaaggccgcgttgctggcgtttttccataggctccg |
| | | cccccctgacgagcatcacaaaaatcgacgctcaagtcagaggtggcgaaacccgacaggactat |
| | | aaagataccaggcgtttccccctggaagctccctcgtgcgctctcctgttccgaccctgccgctt |
| | | accggatacctgtccgcctttctcccttcgggaagcgtggcgctttctcatagctcacgctgtag |
| | | gtatctcagttcggtgtaggtcgttcgctccaagctgggctgtgtgcacgaaccccccgttcagc |
| | | ccgaccgctgcgccttatccggtaactatcgtcttgagtccaacccggtaagacacgacttatcg |
| | | ccactggcagcagccactggtaacaggattagcagagcgaggtatgtaggcggtgctacagagtt |
| | | cttgaagtggtggcctaactacggctacactagaagaacagtatttggtatctgcgctctgctga |
| | | agccagttaccttcggaaaaagagttggtagctcttgatccggcaaacaaaccaccgctggtagc |
| | | ggtggtttttttgtttgcaagcagcagattacgcgcagaaaaaaaggatctcaagaagatccttt |
| | | gatcttttctacggggtctgacgctcagtggaacgaaaactcacgttaagggattttggtcatga |
| | | gattatcaaaaaggatcttcacctagatccttttaaattaaaaatgaagttttaaatcaatctaa |
| | | agtatatatgagtaaacttggtctgacagttaccaatgcttaatcagtgaggcacctatctcagc |
| | | gatctgtctatttcgttcatccatagttgcctgactcgggggggggggggctgaggtctgcctcg |
| | | tgaagaaggtgttgctgactcataccaggcctgaatcgccccatcatccagccagaaagtgaggg |
| | | agccacggttgatgagagctttgttgtaggtggaccagttggtgattttgaacttttgctttgcc |
| | | acggaacggtctgcgttgtcgggaagatgcgtgatctgatccttcaactcagcaaaagttcgatt |
| | | tattcaacaaagccgccgtcccgtcaagtcagcgtaatgctctgccagtgttacaaccaattaac |
| | | caattctgattagaaaaactcatcgagcatcaaatgaaactgcaatttattcatatcaggattat |
| | | caataccatatttttgaaaaagccgtttctgtaatgaaggagaaaactcaccgaggcagttccat |
| | | aggatggcaagatcctggtatcggtctgcgattccgactcgtccaacatcaatacaacctattaa |
| | | tttcccctcgtcaaaaataaggttatcaagtgagaaatcaccatgagtgacgactgaatccggtg |
| | | agaatggcaaaagcttatgcatttctttccagacttgttcaacaggccagccattacgctcgtca |
| | | tcaaaatcactcgcatcaaccaaaccgttattcattcgtgattgcgcctgagcgagacgaaatac |
| | | gcgatcgctgttaaaaggacaattacaaacaggaatcgaatgcaaccggcgcaggaacactgcca |
| | | gcgcatcaacaatattttcacctgaatcaggatattcttctaatacctggaatgctgttttcccg |
| | | gggatcgcagtggtgagtaaccatgcatcatcaggagtacggataaaatgcttgatggtcggaag |
| | | aggcataaattccgtcagccagtttagtctgaccatctcatctgtaacatcattggcaacgctac |
| | | ctttgccatgtttcagaaacaactctggcgcatcgggcttcccatacaatcgatagattgtcgca |
| | | cctgattgcccgacattatcgcgagcccatttatacccatataaatcagcatccatgttggaatt |
| | | taatcgcggcctcgagcaagacgtttcccgttgaatatggctcataacaccccttgtattactgt |
| | | ttatgtaagcagacagttttattgttcatgatgatatatttttatcttgtgcaatgtaacatcag |
| | | agattttgagacacaacgtggctttccccccccccccattattgaagcatttatcagggttattg |
| | | tctcatgagcggatacatatttgaatgtatttagaaaaataaacaaataggggttccgcgcacat |
| | | ttccccgaaaagtgccacctgacgtctaagaaaccattattatcatgacattaacctataaaaat |
| | | aggcgtatcacgaggccctttcgtctcgcgcgtttcggtgatgacggtgaaaacctctgacacat |
| | | gcagctcccggagacggtcacagcttgtctgtaagcggatgccgggagcagacaagcccgtcagg |
| | | gcgcgtcagcgggtgttggcgggtgtcggggctggcttaactatgcggcatcagagcagattgta |
| | | ctgagagtgcaccatatgcggtgtgaaataccgcacagatgcgtaaggagaaaataccgcatcag |
| | | attggctat |
| 22 | Vector expressing | tggccattgcatacgttgtatccatatcataatatgtacatttatattggctcatgtccaacatt |
| | hGNE2 and | accgccatgttgacattgattattgactagttattaatagtaatcaattacggggtcattagttc |
| | knockdown M743T | atagcccatatatggagttccgcgttacataacttacggtaaatggcccgcctggctgaccgccc |
| | hGNE2 mutation in | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | (bi-shRNA is | atatgccaagtacgccccctattgacgtcaatgacggtaaatggcccgcctggcattatgcccag |
| | in front of the | tacatgaccttatgggactttcctacttggcagtacatctacgtattagtcatcgctattaccat |
| | hGNE2 mRNA | ggtgatgcggttttggcagtacatcaatgggcgtggatagcggtttgactcacggggatttccaa |
| | unit; | gtctccaccccattgacgtcaatgggagtttgttttggcaccaaaatcaacgggactttccaaaa |
| | sequence of bi- | tgtcgtaacaactccgccccattgacgcaaatgggcggtaggcgtgtacggtgggaggtctatat |
| | shRNA is in bold) | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgactttcttccccattagggattatgctgaatttgtatggtttatagttgtta |
| | | gagtttgaggtgttaattctaattatctatttcaaatttagcaggaaaaaagagaacatcacctt |
| | | gtaaaactgaagattgtgaccagtcagaataatgtctgctgggtgctgccagcacggtagatatg |
| | | tgcatctaccgtgctggcagcacccagcagcattatggtgacagctgcctcgggaagccaagttg |
| | | ggctttaaagtgcagggcctgctgatgttgagtgctttttgttcatactgtgtgcgaccatcatc |
| | | gtagtgaagtagattagcatctaccgtgctggcagcacccagcagcataagaagttatgtattca |
| | | tccaataattcaagccaagcaagtatataggtgttttaatagtttttgttttggggattgtgacc |
| | | agaagattttgaaaattaaatattactgaagatttcgacttccactgttaaatgtacaagataca |
| | | tgaaatattaaagaaaatgtgtaactttttgtgtaaatacatcttgttcaccgtcgtcgacggta |
| | | tcgataagcttgatatcgaattcgcccttatggagaagaatggaaataaccgaaagctgcgggtt |
| | | tgtgttgctacttgtaaccgtgcagattattctaaacttgccccgatcatgtttggcattaaaac |
| | | cgaacctgagttctttgaacttgatgttgtggtacttggctctcacctgatagatgactatggaa |
| | | atacatatcgaatgattgaacaagatgactttgacattaacaccaggctacacacaattgtgagg |
| | | ggagaagatgaggcagccatggtggagtcagtaggcctggccctagtgaagctgccagatgtcct |
| | | taatcgcctgaagcctgatatcatgattgttcatggagacaggtttgatgccctggctctggcca |
| | | catctgctgccttgatgaacatccgaatccttcacattgaaggtggggaagtcagtgggaccatt |
| | | gatgactctatcagacatgccataacaaaactggctcattatcatgtgtgctgcacccgcagtgc |
| | | agagcagcacctgatatccatgtgtgaggaccatgatcgcatccttttggcaggctgcccttcct |
| | | atgacaaacttctctcagccaagaacaaagactacatgagcatcattcgcatgtggctaggtgat |
| | | gatgtaaaatctaaagattacattgttgcactacagcaccctgtgaccactgacattaagcattc |
| | | cataaaaatgtttgaattaacattggatgcacttatctcatttaacaagcggaccctagtcctgt |
| | | ttccaaatattgacgcagggagcaaagagatggttcgagtgatgcggaagaagggcattgagcat |
| | | catcccaactttcgtgcagttaaacacgtcccatttgaccagtttatacagttggttgcccatgc |
| | | tggctgtatgattgggaacagcagctgtggggttcgagaagttggagcttttggaacacctgtga |
| | | tcaacctgggaacacgtcagattggaagagaaacaggggagaatgttcttcatgtccgggatgct |
| | | gacacccaagacaaaatattgcaagcactgcaccttcagtttggtaaacagtacccttgttcaaa |
| | | gatatatggggatggaaatgctgttccaaggattttgaagtttctcaaatctatcgatcttcaag |
| | | agccactgcaaaagaaattctgctttcctcctgtgaaggagaatatctctcaagatattgaccat |
| | | attcttgaaactctaagtgccttggccgttgatcttggcgggacgaacctccgagttgcaatagt |
| | | cagcatgaagggtgaaatagttaagaagtatactcagttcaatcctaaaacctatgaagagagga |
| | | ttaatttaatcctacagatgtgtgtggaagctgcagcagaagctgtaaaactgaactgcagaatt |
| | | ttgggagtaggcatttccacaggtggccgtgtaaatcctcgggaaggaattgtgctgcattcaac |
| | | caaactgatccaagagtggaactctgtggaccttaggacccccctttctgacactttgcatctcc |
| | | ctgtgtgggtagacaatgatggcaactgtgctgccctggcggaaaggaaatttggccaaggaaag |
| | | ggactggaaaactttgttacacttatcacaggcacaggaatcggtggtggaattatccatcagca |
| | | tgaattgatccacggaagctccttctgtgctgcagaactgggccaccttgttgtgtctctggatg |
| | | ggcctgattgttcctgtggaagccatgggtgcattgaagcatacgcctctggaatggccttgcag |
| | | agggaggcaaaaaagctccatgatgaggacctgctcttggtggaagggatgtcagtgccaaaaga |
| | | tgaggctgtgggtgcgctccatctcatccaagctgcgaaacttggcaatgcgaaggcccagagca |
| | | tcctaagaacagctggaacagctttgggtcttggggttgtgaacatcctccataccatgaatccc |
| | | tcccttgtgatcctctccggagtcctggccagtcactatatccacattgtcaaagacgtcattcg |
| | | ccagcaggccttgtcctccgtgcaggacgtggatgtggtggtttcggatttggttgaccccgccc |
| | | tgctgggtgctgccagcatggttctggactacacaacacgcaggatctactagaagggcgaattc |
| | | acgtgggcccggtaccgtatactctagagcggccgcggatccagatctttttccctctgccaaaa |
| | | attatggggacatcatgaagccccttgagcatctgacttctggctaataaaggaaatttattttc |
| | | attgcaatagtgtgttggaattttttgtgtctctcactcggaaggacatatgggagggcaaatca |
| | | tttaaaacatcagaatgagtatttggtttagagtttggcaacatatgcccattcttccgcttcct |
| | | cgctcactgactcgctgcgctcggtcgttcggctgcggcgagcggtatcagctcactcaaaggcg |
| | | gtaatacggttatccacagaatcaggggataacgcaggaaagaacatgtgagcaaaaggccagca |
| | | aaaggccaggaaccgtaaaaaggccgcgttgctggcgtttttccataggctccgcccccctgacg |
| | | agcatcacaaaaatcgacgctcaagtcagaggtggcgaaacccgacaggactataaagataccag |
| | | gcgtttccccctggaagctccctcgtgcgctctcctgttccgaccctgccgcttaccggatacct |
| | | gtccgcctttctcccttcgggaagcgtggcgctttctcatagctcacgctgtaggtatctcagtt |
| | | cggtgtaggtcgttcgctccaagctgggctgtgtgcacgaaccccccgttcagcccgaccgctgc |
| | | gccttatccggtaactatcgtcttgagtccaacccggtaagacacgacttatcgccactggcagc |
| | | agccactggtaacaggattagcagagcgaggtatgtaggcggtgctacagagttcttgaagtggt |
| | | ggcctaactacggctacactagaagaacagtatttggtatctgcgctctgctgaagccagttacc |
| | | ttcggaaaaagagttggtagctcttgatccggcaaacaaaccaccgctggtagcggtggtttttt |
| | | tgtttgcaagcagcagattacgcgcagaaaaaaaggatctcaagaagatcctttgatcttttcta |
| | | cggggtctgacgctcagtggaacgaaaactcacgttaagggattttggtcatgagattatcaaaa |
| | | aggatcttcacctagatccttttaaattaaaaatgaagttttaaatcaatctaaagtatatatga |
| | | gtaaacttggtctgacagttaccaatgcttaatcagtgaggcacctatctcagcgatctgtctat |
| | | ttcgttcatccatagttgcctgactcggggggggggggcgctgaggtctgcctcgtgaagaaggt |
| | | gttgctgactcataccaggcctgaatcgccccatcatccagccagaaagtgagggagccacggtt |
| | | gatgagagctttgttgtaggtggaccagttggtgattttgaacttttgctttgccacggaacggt |
| | | ctgcgttgtcgggaagatgcgtgatctgatccttcaactcagcaaaagttcgatttattcaacaa |
| | | agccgccgtcccgtcaagtcagcgtaatgctctgccagtgttacaaccaattaaccaattctgat |
| | | tagaaaaactcatcgagcatcaaatgaaactgcaatttattcatatcaggattatcaataccata |
| | | tttttgaaaaagccgtttctgtaatgaaggagaaaactcaccgaggcagttccataggatggcaa |
| | | gatcctggtatcggtctgcgattccgactcgtccaacatcaatacaacctattaatttcccctcg |
| | | tcaaaaataaggttatcaagtgagaaatcaccatgagtgacgactgaatccggtgagaatggcaa |
| | | aagcttatgcatttctttccagacttgttcaacaggccagccattacgctcgtcatcaaaatcac |
| | | tcgcatcaaccaaaccgttattcattcgtgattgcgcctgagcgagacgaaatacgcgatcgctg |
| | | ttaaaaggacaattacaaacaggaatcgaatgcaaccggcgcaggaacactgccagcgcatcaac |
| | | aatattttcacctgaatcaggatattcttctaatacctggaatgctgttttcccggggatcgcag |
| | | tggtgagtaaccatgcatcatcaggagtacggataaaatgcttgatggtcggaagaggcataaat |
| | | tccgtcagccagtttagtctgaccatctcatctgtaacatcattggcaacgctacctttgccatg |
| | | tttcagaaacaactctggcgcatcgggcttcccatacaatcgatagattgtcgcacctgattgcc |
| | | cgacattatcgcgagcccatttatacccatataaatcagcatccatgttggaatttaatcgcggc |
| | | ctcgagcaagacgtttcccgttgaatatggctcataacaccccttgtattactgtttatgtaagc |
| | | agacagttttattgttcatgatgatatatttttatcttgtgcaatgtaacatcagagattttgag |
| | | acacaacgtggctttccccccccccccattattgaagcatttatcagggttattgtctcatgagc |
| | | ggatacatatttgaatgtatttagaaaaataaacaaataggggttccgcgcacatttccccgaaa |
| | | agtgccacctgacgtctaagaaaccattattatcatgacattaacctataaaaataggcgtatca |
| | | cgaggccctttcgtctcgcgcgtttcggtgatgacggtgaaaacctctgacacatgcagctcccg |
| | | gagacggtcacagcttgtctgtaagcggatgccgggagcagacaagcccgtcagggcgcgtcagc |
| | | gggtgttggcgggtgtcggggctggcttaactatgcggcatcagagcagattgtactgagagtgc |
| | | accatatgcggtgtgaaataccgcacagatgcgtaaggagaaaataccgcatcagattggctat |
| 23 | Vector expressing | tggccattgcatacgttgtatccatatcataatatgtacatttatattggctcatgtccaacatt |
| | hGNE2 and | accgccatgttgacattgattattgactagttattaatagtaatcaattacggggtcattagttc |
| | knockdown M743T | atagcccatatatggagttccgcgttacataacttacggtaaatggcccgcctggctgaccgccc |
| | hGNE2 mutation in | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | (bi-shRNA is behind | atatgccaagtacgccccctattgacgtcaatgacggtaaatggcccgcctggcattatgcccag |
| | the hGNE2 mRNA | tacatgaccttatgggactttcctacttggcagtacatctacgtattagtcatcgctattaccat |
| | unit; | ggtgatgcggttttggcagtacatcaatgggcgtggatagcggtttgactcacggggatttccaa |
| | sequence of bi- | gtctccaccccattgacgtcaatgggagtttgttttggcaccaaaatcaacgggactttccaaaa |
| | shRNA is in bold) | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacgtaagtaccgcctatagactctataggcacacccctttggctcttatgca |
| | | tgctatactgtttttggcttggggcctatacacccccgcttccttatgctataggtgatggtata |
| | | gcttagcctataggtgtgggttattgaccattattgaccactccaacggtggagggcagtgtagt |
| | | ctgagcagtactcgttgctgccgcgcgcgccaccagacataatagctgacagactaacagactgt |
| | | tcctttccatgggtcttttctgcagtcaccgtcgtcgacggtatcgataagcttgatatcgaatt |
| | | cgcccttatggagaagaatggaaataaccgaaagctgcgggtttgtgttgctacttgtaaccgtg |
| | | cagattattctaaacttgccccgatcatgtttggcattaaaaccgaacctgagttctttgaactt |
| | | gatgttgtggtacttggctctcacctgatagatgactatggaaatacatatcgaatgattgaaca |
| | | agatgactttgacattaacaccaggctacacacaattgtgaggggagaagatgaggcagccatgg |
| | | tggagtcagtaggcctggccctagtgaagctgccagatgtccttaatcgcctgaagcctgatatc |
| | | atgattgttcatggagacaggtttgatgccctggctctggccacatctgctgccttgatgaacat |
| | | ccgaatccttcacattgaaggtggggaagtcagtgggaccattgatgactctatcagacatgcca |
| | | taacaaaactggctcattatcatgtgtgctgcacccgcagtgcagagcagcacctgatatccatg |
| | | tgtgaggaccatgatcgcatccttttggcaggctgcccttcctatgacaaacttctctcagccaa |
| | | gaacaaagactacatgagcatcattcgcatgtggctaggtgatgatgtaaaatctaaagattaca |
| | | ttgttgcactacagcaccctgtgaccactgacattaagcattccataaaaatgtttgaattaaca |
| | | ttggatgcacttatctcatttaacaagcggaccctagtcctgtttccaaatattgacgcagggag |
| | | caaagagatggttcgagtgatgcggaagaagggcattgagcatcatcccaactttcgtgcagtta |
| | | aacacgtcccatttgaccagtttatacagttggttgcccatgctggctgtatgattgggaacagc |
| | | agctgtggggttcgagaagttggagcttttggaacacctgtgatcaacctgggaacacgtcagat |
| | | tggaagagaaacaggggagaatgttcttcatgtccgggatgctgacacccaagacaaaatattgc |
| | | aagcactgcaccttcagtttggtaaacagtacccttgttcaaagatatatggggatggaaatgct |
| | | gttccaaggattttgaagtttctcaaatctatcgatcttcaagagccactgcaaaagaaattctg |
| | | ctttcctcctgtgaaggagaatatctctcaagatattgaccatattcttgaaactctaagtgcct |
| | | tggccgttgatcttggcgggacgaacctccgagttgcaatagtcagcatgaagggtgaaatagtt |
| | | aagaagtatactcagttcaatcctaaaacctatgaagagaggattaatttaatcctacagatgtg |
| | | tgtggaagctgcagcagaagctgtaaaactgaactgcagaattttgggagtaggcatttccacag |
| | | gtggccgtgtaaatcctcgggaaggaattgtgctgcattcaaccaaactgatccaagagtggaac |
| | | tctgtggaccttaggacccccctttctgacactttgcatctccctgtgtgggtagacaatgatgg |
| | | caactgtgctgccctggcggaaaggaaatttggccaaggaaagggactggaaaactttgttacac |
| | | ttatcacaggcacaggaatcggtggtggaattatccatcagcatgaattgatccacggaagctcc |
| | | ttctgtgctgcagaactgggccaccttgttgtgtctctggatgggcctgattgttcctgtggaag |
| | | ccatgggtgcattgaagcatacgcctctggaatggccttgcagagggaggcaaaaaagctccatg |
| | | atgaggacctgctcttggtggaagggatgtcagtgccaaaagatgaggctgtgggtgcgctccat |
| | | ctcatccaagctgcgaaacttggcaatgcgaaggcccagagcatcctaagaacagctggaacagc |
| | | tttgggtcttggggttgtgaacatcctccataccatgaatccctcccttgtgatcctctccggag |
| | | tcctggccagtcactatatccacattgtcaaagacgtcattcgccagcaggccttgtcctccgtg |
| | | caggacgtggatgtggtggtttcggatttggttgaccccgccctgctgggtgctgccagcatggt |
| | | tctggactacacaacacgcaggatctactagaagggcgaattcacgtgggcccggtaccgtatac |
| | | tctagagcggccgcggtttcttccccattagggattatgctgaatttgtatggtttatagttgtt |
| | | agagtttgaggtgttaattctaattatctatttcaaatttagcaggaaaaaagagaacatcacct |
| | | tgtaaaactgaagattgtgaccagtcagaataatgtctgctgggtgctgccagcacggtagatat |
| | | gtgcatctaccgtgctggcagcacccagcagcattatggtgacagctgcctcgggaagccaagtt |
| | | gggctttaaagtgcagggcctgctgatgttgagtgctttttgttcatactgtgtgcgaccatcat |
| | | cgtagtgaagtagattagcatctaccgtgctggcagcacccagcagcataagaagttatgtattc |
| | | atccaataattcaagccaagcaagtatataggtgttttaatagtttttgttttggggattgtgac |
| | | cagaagattttgaaaattaaatattactgaagatttcgacttccactgttaaatgtacaagatac |
| | | atgaaatattaaagaaaatgtgtaactttttgtgtaaatacatcttgtatccagatctttttccc |
| | | tctgccaaaaattatggggacatcatgaagccccttgagcatctgacttctggctaataaaggaa |
| | | atttattttcattgcaatagtgtgttggaattttttgtgtctctcactcggaaggacatatggga |
| | | gggcaaatcatttaaaacatcagaatgagtatttggtttagagtttggcaacatatgcccattct |
| | | tccgcttcctcgctcactgactcgctgcgctcggtcgttcggctgcggcgagcggtatcagctca |
| | | ctcaaaggcggtaatacggttatccacagaatcaggggataacgcaggaaagaacatgtgagcaa |
| | | aaggccagcaaaaggccaggaaccgtaaaaaggccgcgttgctggcgtttttccataggctccgc |
| | | ccccctgacgagcatcacaaaaatcgacgctcaagtcagaggtggcgaaacccgacaggactata |
| | | aagataccaggcgtttccccctggaagctccctcgtgcgctctcctgttccgaccctgccgctta |
| | | ccggatacctgtccgcctttctcccttcgggaagcgtggcgctttctcatagctcacgctgtagg |
| | | tatctcagttcggtgtaggtcgttcgctccaagctgggctgtgtgcacgaaccccccgttcagcc |
| | | cgaccgctgcgccttatccggtaactatcgtcttgagtccaacccggtaagacacgacttatcgc |
| | | cactggcagcagccactggtaacaggattagcagagcgaggtatgtaggcggtgctacagagttc |
| | | ttgaagtggtggcctaactacggctacactagaagaacagtatttggtatctgcgctctgctgaa |
| | | gccagttaccttcggaaaaagagttggtagctcttgatccggcaaacaaaccaccgctggtagcg |
| | | gtggtttttttgtttgcaagcagcagattacgcgcagaaaaaaaggatctcaagaagatcctttg |
| | | atcttttctacggggtctgacgctcagtggaacgaaaactcacgttaagggattttggtcatgag |
| | | attatcaaaaaggatcttcacctagatccttttaaattaaaaatgaagttttaaatcaatctaaa |
| | | gtatatatgagtaaacttggtctgacagttaccaatgcttaatcagtgaggcacctatctcagcg |
| | | atctgtctatttcgttcatccatagttgcctgactcgggggggggggggctgaggtctgcctcgt |
| | | gaagaaggtgttgctgactcataccaggcctgaatcgccccatcatccagccagaaagtgaggga |
| | | gccacggttgatgagagctttgttgtaggtggaccagttggtgattttgaacttttgctttgcca |
| | | cggaacggtctgcgttgtcgggaagatgcgtgatctgatccttcaactcagcaaaagttcgattt |
| | | attcaacaaagccgccgtcccgtcaagtcagcgtaatgctctgccagtgttacaaccaattaacc |
| | | aattctgattagaaaaactcatcgagcatcaaatgaaactgcaatttattcatatcaggattatc |
| | | aataccatatttttgaaaaagccgtttctgtaatgaaggagaaaactcaccgaggcagttccata |
| | | ggatggcaagatcctggtatcggtctgcgattccgactcgtccaacatcaatacaacctattaat |
| | | ttcccctcgtcaaaaataaggttatcaagtgagaaatcaccatgagtgacgactgaatccggtga |
| | | gaatggcaaaagcttatgcatttctttccagacttgttcaacaggccagccattacgctcgtcat |
| | | caaaatcactcgcatcaaccaaaccgttattcattcgtgattgcgcctgagcgagacgaaatacg |
| | | cgatcgctgttaaaaggacaattacaaacaggaatcgaatgcaaccggcgcaggaacactgccag |
| | | cgcatcaacaatattttcacctgaatcaggatattcttctaatacctggaatgctgttttcccgg |
| | | ggatcgcagtggtgagtaaccatgcatcatcaggagtacggataaaatgcttgatggtcggaaga |
| | | ggcataaattccgtcagccagtttagtctgaccatctcatctgtaacatcattggcaacgctacc |
| | | tttgccatgtttcagaaacaactctggcgcatcgggcttcccatacaatcgatagattgtcgcac |
| | | ctgattgcccgacattatcgcgagcccatttatacccatataaatcagcatccatgttggaattt |
| | | aatcgcggcctcgagcaagacgtttcccgttgaatatggctcataacaccccttgtattactgtt |
| | | tatgtaagcagacagttttattgttcatgatgatatatttttatcttgtgcaatgtaacatcaga |
| | | gattttgagacacaacgtggctttccccccccccccattattgaagcatttatcagggttattgt |
| | | ctcatgagcggatacatatttgaatgtatttagaaaaataaacaaataggggttccgcgcacatt |
| | | tccccgaaaagtgccacctgacgtctaagaaaccattattatcatgacattaacctataaaaata |
| | | ggcgtatcacgaggccctttcgtctcgcgcgtttcggtgatgacggtgaaaacctctgacacatg |
| | | cagctcccggagacggtcacagcttgtctgtaagcggatgccgggagcagacaagcccgtcaggg |
| | | cgcgtcagcgggtgttggcgggtgtcggggctggcttaactatgcggcatcagagcagattgtac |
| | | tgagagtgcaccatatgcggtgtgaaataccgcacagatgcgtaaggagaaaataccgcatcaga |
| | | ttggctat |
| 24 | Vector expressing | tggccattgcatacgttgtatccatatcataatatgtacatttatattggctcatgtccaacatt |
| | Strep II | accgccatgttgacattgattattgactagttattaatagtaatcaattacggggtcattagttc |
| | tagged hGNE2 | atagcccatatatggagttccgcgttacataacttacggtaaatggcccgcctggctgaccgccc |
| | pUMVC3 backbone | aacgacccccgcccattgacgtcaataatgacgtatgttcccatagtaacgccaatagggacttt |
| | | ccattgacgtcaatgggtggagtatttacggtaaactgcccacttggcagtacatcaagtgtatc |
| | | atatgccaagtacgccccctattgacgtcaatgacggtaaatggcccgcctggcattatgcccag |
| | | tacatgaccttatgggactttcctacttggcagtacatctacgtattagtcatcgctattaccat |
| | | ggtgatgcggttttggcagtacatcaatgggcgtggatagcggtttgactcacggggatttccaa |
| | | gtctccaccccattgacgtcaatgggagtttgttttggcaccaaaatcaacgggactttccaaaa |
| | | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacgtaagtaccgcctatagactctataggcacacccctttggctcttatgca |
| | | tgctatactgtttttggcttggggcctatacacccccgcttccttatgctataggtgatggtata |
| | | gcttagcctataggtgtgggttattgaccattattgaccactccaacggtggagggcagtgtagt |
| | | ctgagcagtactcgttgctgccgcgcgcgccaccagacataatagctgacagactaacagactgt |
| | | tcctttccatgggtcttttctgcagtcaccgtcgtcgacggtatcgataagcttgatatcgaatt |
| | | cgcccttatgtggagccatccacagtttgagaagatggagaagaatggaaataaccgaaagctgc |
| | | gggtttgtgttgctacttgtaaccgtgcagattattctaaacttgccccgatcatgtttggcatt |
| | | aaaaccgaacctgagttctttgaacttgatgttgtggtacttggctctcacctgatagatgacta |
| | | tggaaatacatatcgaatgattgaacaagatgactttgacattaacaccaggctacacacaattg |
| | | tgaggggagaagatgaggcagccatggtggagtcagtaggcctggccctagtgaagctgccagat |
| | | gtccttaatcgcctgaagcctgatatcatgattgttcatggagacaggtttgatgccctggctct |
| | | ggccacatctgctgccttgatgaacatccgaatccttcacattgaaggtggggaagtcagtggga |
| | | ccattgatgactctatcagacatgccataacaaaactggctcattatcatgtgtgctgcacccgc |
| | | agtgcagagcagcacctgatatccatgtgtgaggaccatgatcgcatccttttggcaggctgccc |
| | | ttcctatgacaaacttctctcagccaagaacaaagactacatgagcatcattcgcatgtggctag |
| | | gtgatgatgtaaaatctaaagattacattgttgcactacagcaccctgtgaccactgacattaag |
| | | cattccataaaaatgtttgaattaacattggatgcacttatctcatttaacaagcggaccctagt |
| | | cctgtttccaaatattgacgcagggagcaaagagatggttcgagtgatgcggaagaagggcattg |
| | | agcatcatcccaactttcgtgcagttaaacacgtcccatttgaccagtttatacagttggttgcc |
| | | catgctggctgtatgattgggaacagcagctgtggggttcgagaagttggagcttttggaacacc |
| | | tgtgatcaacctgggaacacgtcagattggaagagaaacaggggagaatgttcttcatgtccggg |
| | | atgctgacacccaagacaaaatattgcaagcactgcaccttcagtttggtaaacagtacccttgt |
| | | tcaaagatatatggggatggaaatgctgttccaaggattttgaagtttctcaaatctatcgatct |
| | | tcaagagccactgcaaaagaaattctgctttcctcctgtgaaggagaatatctctcaagatattg |
| | | accatattcttgaaactctaagtgccttggccgttgatcttggcgggacgaacctccgagttgca |
| | | atagtcagcatgaagggtgaaatagttaagaagtatactcagttcaatcctaaaacctatgaaga |
| | | gaggattaatttaatcctacagatgtgtgtggaagctgcagcagaagctgtaaaactgaactgca |
| | | gaattttgggagtaggcatttccacaggtggccgtgtaaatcctcgggaaggaattgtgctgcat |
| | | tcaaccaaactgatccaagagtggaactctgtggaccttaggacccccctttctgacactttgca |
| | | tctccctgtgtgggtagacaatgatggcaactgtgctgccctggcggaaaggaaatttggccaag |
| | | gaaagggactggaaaactttgttacacttatcacaggcacaggaatcggtggtggaattatccat |
| | | cagcatgaattgatccacggaagctccttctgtgctgcagaactgggccaccttgttgtgtctct |
| | | ggatgggcctgattgttcctgtggaagccatgggtgcattgaagcatacgcctctggaatggcct |
| | | tgcagagggaggcaaaaaagctccatgatgaggacctgctcttggtggaagggatgtcagtgcca |
| | | aaagatgaggctgtgggtgcgctccatctcatccaagctgcgaaacttggcaatgcgaaggccca |
| | | gagcatcctaagaacagctggaacagctttgggtcttggggttgtgaacatcctccataccatga |
| | | atccctcccttgtgatcctctccggagtcctggccagtcactatatccacattgtcaaagacgtc |
| | | attcgccagcaggccttgtcctccgtgcaggacgtggatgtggtggtttcggatttggttgaccc |
| | | cgccctgctgggtgctgccagcatggttctggactacacaacacgcaggatctactagaagggcg |
| | | aattcacgtgggcccggtaccgtatactctagagcggccgcggatccagatctttttccctctgc |
| | | caaaaattatggggacatcatgaagccccttgagcatctgacttctggctaataaaggaaattta |
| | | ttttcattgcaatagtgtgttggaattttttgtgtctctcactcggaaggacatatgggagggca |
| | | aatcatttaaaacatcagaatgagtatttggtttagagtttggcaacatatgcccattcttccgc |
| | | ttcctcgctcactgactcgctgcgctcggtcgttcggctgcggcgagcggtatcagctcactcaa |
| | | aggcggtaatacggttatccacagaatcaggggataacgcaggaaagaacatgtgagcaaaaggc |
| | | cagcaaaaggccaggaaccgtaaaaaggccgcgttgctggcgtttttccataggctccgcccccc |
| | | tgacgagcatcacaaaaatcgacgctcaagtcagaggtggcgaaacccgacaggactataaagat |
| | | accaggcgtttccccctggaagctccctcgtgcgctctcctgttccgaccctgccgcttaccgga |
| | | tacctgtccgcctttctcccttcgggaagcgtggcgctttctcatagctcacgctgtaggtatct |
| | | cagttcggtgtaggtcgttcgctccaagctgggctgtgtgcacgaaccccccgttcagcccgacc |
| | | gctgcgccttatccggtaactatcgtcttgagtccaacccggtaagacacgacttatcgccactg |
| | | gcagcagccactggtaacaggattagcagagcgaggtatgtaggcggtgctacagagttcttgaa |
| | | gtggtggcctaactacggctacactagaagaacagtatttggtatctgcgctctgctgaagccag |
| | | ttaccttcggaaaaagagttggtagctcttgatccggcaaacaaaccaccgctggtagcggtggt |
| | | ttttttgtttgcaagcagcagattacgcgcagaaaaaaaggatctcaagaagatcctttgatctt |
| | | ttctacggggtctgacgctcagtggaacgaaaactcacgttaagggattttggtcatgagattat |
| | | caaaaaggatcttcacctagatccttttaaattaaaaatgaagttttaaatcaatctaaagtata |
| | | tatgagtaaacttggtctgacagttaccaatgcttaatcagtgaggcacctatctcagcgatctg |
| | | tctatttcgttcatccatagttgcctgactcggggggggggggcgctgaggtctgcctcgtgaag |
| | | aaggtgttgctgactcataccaggcctgaatcgccccatcatccagccagaaagtgagggagcca |
| | | cggttgatgagagctttgttgtaggtggaccagttggtgattttgaacttttgctttgccacgga |
| | | acggtctgcgttgtcgggaagatgcgtgatctgatccttcaactcagcaaaagttcgatttattc |
| | | aacaaagccgccgtcccgtcaagtcagcgtaatgctctgccagtgttacaaccaattaaccaatt |
| | | ctgattagaaaaactcatcgagcatcaaatgaaactgcaatttattcatatcaggattatcaata |
| | | ccatatttttgaaaaagccgtttctgtaatgaaggagaaaactcaccgaggcagttccataggat |
| | | ggcaagatcctggtatcggtctgcgattccgactcgtccaacatcaatacaacctattaatttcc |
| | | cctcgtcaaaaataaggttatcaagtgagaaatcaccatgagtgacgactgaatccggtgagaat |
| | | ggcaaaagcttatgcatttctttccagacttgttcaacaggccagccattacgctcgtcatcaaa |
| | | atcactcgcatcaaccaaaccgttattcattcgtgattgcgcctgagcgagacgaaatacgcgat |
| | | cgctgttaaaaggacaattacaaacaggaatcgaatgcaaccggcgcaggaacactgccagcgca |
| | | tcaacaatattttcacctgaatcaggatattcttctaatacctggaatgctgttttcccggggat |
| | | cgcagtggtgagtaaccatgcatcatcaggagtacggataaaatgcttgatggtcggaagaggca |
| | | taaattccgtcagccagtttagtctgaccatctcatctgtaacatcattggcaacgctacctttg |
| | | ccatgtttcagaaacaactctggcgcatcgggcttcccatacaatcgatagattgtcgcacctga |
| | | ttgcccgacattatcgcgagcccatttatacccatataaatcagcatccatgttggaatttaatc |
| | | gcggcctcgagcaagacgtttcccgttgaatatggctcataacaccccttgtattactgtttatg |
| | | taagcagacagttttattgttcatgatgatatatttttatcttgtgcaatgtaacatcagagatt |
| | | ttgagacacaacgtggctttccccccccccccattattgaagcatttatcagggttattgtctca |
| | | tgagcggatacatatttgaatgtatttagaaaaataaacaaataggggttccgcgcacatttccc |
| | | cgaaaagtgccacctgacgtctaagaaaccattattatcatgacattaacctataaaaataggcg |
| | | tatcacgaggccctttcgtctcgcgcgtttcggtgatgacggtgaaaacctctgacacatgcagc |
| | | tcccggagacggtcacagcttgtctgtaagcggatgccgggagcagacaagcccgtcagggcgcg |
| | | tcagcgggtgttggcgggtgtcggggctggcttaactatgcggcatcagagcagattgtactgag |
| | | agtgcaccatatgcggtgtgaaataccgcacagatgcgtaaggagaaaataccgcatcagattgg |
| | | ctat |
| 25 | Vector expressing | tggccattgcatacgttgtatccatatcataatatgtacatttatattggctcatgtccaacatt |
| | Strep II | accgccatgttgacattgattattgactagttattaatagtaatcaattacggggtcattagttc |
| | tagged M743T | atagcccatatatggagttccgcgttacataacttacggtaaatggcccgcctggctgaccgccc |
| | hGNE2 mutant | aacgacccccgcccattgacgtcaataatgacgtatgttcccatagtaacgccaatagggacttt |
| | pUMVC3 backbone | ccattgacgtcaatgggtggagtatttacggtaaactgcccacttggcagtacatcaagtgtatc |
| | | atatgccaagtacgccccctattgacgtcaatgacggtaaatggcccgcctggcattatgcccag |
| | | tacatgaccttatgggactttcctacttggcagtacatctacgtattagtcatcgctattaccat |
| | | ggtgatgcggttttggcagtacatcaatgggcgtggatagcggtttgactcacggggatttccaa |
| | | gtctccaccccattgacgtcaatgggagtttgttttggcaccaaaatcaacgggactttccaaaa |
| | | tgtcgtaacaactccgccccattgacgcaaatgggcggtaggcgtgtacggtgggaggtctatat |
| | | aagcagagctcgtttagtgaaccgtcagatcgcctggagacgccatccacgctgttttgacctcc |
| | | atagaagacaccgggaccgatccagcctccgcggccgggaacggtgcattggaacgcggattccc |
| | | cgtgccaagagtgacgtaagtaccgcctatagactctataggcacacccctttggctcttatgca |
| | | tgctatactgtttttggcttggggcctatacacccccgcttccttatgctataggtgatggtata |
| | | gcttagcctataggtgtgggttattgaccattattgaccactccaacggtggagggcagtgtagt |
| | | ctgagcagtactcgttgctgccgcgcgcgccaccagacataatagctgacagactaacagactgt |
| | | tcctttccatgggtcttttctgcagtcaccgtcgtcgacggtatcgataagcttgatatcgaatt |
| | | cgcccttatgtggagccatccacagtttgagaagatggagaagaatggaaataaccgaaagctgc |
| | | gggtttgtgttgctacttgtaaccgtgcagattattctaaacttgccccgatcatgtttggcatt |
| | | aaaaccgaacctgagttctttgaacttgatgttgtggtacttggctctcacctgatagatgacta |
| | | tggaaatacatatcgaatgattgaacaagatgactttgacattaacaccaggctacacacaattg |
| | | tgaggggagaagatgaggcagccatggtggagtcagtaggcctggccctagtgaagctgccagat |
| | | gtccttaatcgcctgaagcctgatatcatgattgttcatggagacaggtttgatgccctggctct |
| | | ggccacatctgctgccttgatgaacatccgaatccttcacattgaaggtggggaagtcagtggga |
| | | ccattgatgactctatcagacatgccataacaaaactggctcattatcatgtgtgctgcacccgc |
| | | agtgcagagcagcacctgatatccatgtgtgaggaccatgatcgcatccttttggcaggctgccc |
| | | ttcctatgacaaacttctctcagccaagaacaaagactacatgagcatcattcgcatgtggctag |
| | | gtgatgatgtaaaatctaaagattacattgttgcactacagcaccctgtgaccactgacattaag |
| | | cattccataaaaatgtttgaattaacattggatgcacttatctcatttaacaagcggaccctagt |
| | | cctgtttccaaatattgacgcagggagcaaagagatggttcgagtgatgcggaagaagggcattg |
| | | agcatcatcccaactttcgtgcagttaaacacgtcccatttgaccagtttatacagttggttgcc |
| | | catgctggctgtatgattgggaacagcagctgtggggttcgagaagttggagcttttggaacacc |
| | | tgtgatcaacctgggaacacgtcagattggaagagaaacaggggagaatgttcttcatgtccggg |
| | | atgctgacacccaagacaaaatattgcaagcactgcaccttcagtttggtaaacagtacccttgt |
| | | tcaaagatatatggggatggaaatgctgttccaaggattttgaagtttctcaaatctatcgatct |
| | | tcaagagccactgcaaaagaaattctgctttcctcctgtgaaggagaatatctctcaagatattg |
| | | accatattcttgaaactctaagtgccttggccgttgatcttggcgggacgaacctccgagttgca |
| | | atagtcagcatgaagggtgaaatagttaagaagtatactcagttcaatcctaaaacctatgaaga |
| | | gaggattaatttaatcctacagatgtgtgtggaagctgcagcagaagctgtaaaactgaactgca |
| | | gaattttgggagtaggcatttccacaggtggccgtgtaaatcctcgggaaggaattgtgctgcat |
| | | tcaaccaaactgatccaagagtggaactctgtggaccttaggacccccctttctgacactttgca |
| | | tctccctgtgtgggtagacaatgatggcaactgtgctgccctggcggaaaggaaatttggccaag |
| | | gaaagggactggaaaactttgttacacttatcacaggcacaggaatcggtggtggaattatccat |
| | | cagcatgaattgatccacggaagctccttctgtgctgcagaactgggccaccttgttgtgtctct |
| | | ggatgggcctgattgttcctgtggaagccatgggtgcattgaagcatacgcctctggaatggcct |
| | | tgcagagggaggcaaaaaagctccatgatgaggacctgctcttggtggaagggatgtcagtgcca |
| | | aaagatgaggctgtgggtgcgctccatctcatccaagctgcgaaacttggcaatgcgaaggccca |
| | | gagcatcctaagaacagctggaacagctttgggtcttggggttgtgaacatcctccataccatga |
| | | atccctcccttgtgatcctctccggagtcctggccagtcactatatccacattgtcaaagacgtc |
| | | attcgccagcaggccttgtcctccgtgcaggacgtggatgtggtggtttcggatttggttgaccc |
| | | cgccctgctgggtgctgccagcacggttctggactacacaacacgcaggatctactagaagggcg |
| | | aattcacgtgggcccggtaccgtatactctagagcggccgcggatccagatctttttccctctgc |
| | | caaaaattatggggacatcatgaagccccttgagcatctgacttctggctaataaaggaaattta |
| | | ttttcattgcaatagtgtgttggaattttttgtgtctctcactcggaaggacatatgggagggca |
| | | aatcatttaaaacatcagaatgagtatttggtttagagtttggcaacatatgcccattcttccgc |
| | | ttcctcgctcactgactcgctgcgctcggtcgttcggctgcggcgagcggtatcagctcactcaa |
| | | aggcggtaatacggttatccacagaatcaggggataacgcaggaaagaacatgtgagcaaaaggc |
| | | cagcaaaaggccaggaaccgtaaaaaggccgcgttgctggcgtttttccataggctccgcccccc |
| | | tgacgagcatcacaaaaatcgacgctcaagtcagaggtggcgaaacccgacaggactataaagat |
| | | accaggcgtttccccctggaagctccctcgtgcgctctcctgttccgaccctgccgcttaccgga |
| | | tacctgtccgcctttctcccttcgggaagcgtggcgctttctcatagctcacgctgtaggtatct |
| | | cagttcggtgtaggtcgttcgctccaagctgggctgtgtgcacgaaccccccgttcagcccgacc |
| | | gctgcgccttatccggtaactatcgtcttgagtccaacccggtaagacacgacttatcgccactg |
| | | gcagcagccactggtaacaggattagcagagcgaggtatgtaggcggtgctacagagttcttgaa |
| | | gtggtggcctaactacggctacactagaagaacagtatttggtatctgcgctctgctgaagccag |
| | | ttaccttcggaaaaagagttggtagctcttgatccggcaaacaaaccaccgctggtagcggtggt |
| | | ttttttgtttgcaagcagcagattacgcgcagaaaaaaaggatctcaagaagatcctttgatctt |
| | | ttctacggggtctgacgctcagtggaacgaaaactcacgttaagggattttggtcatgagattat |
| | | caaaaaggatcttcacctagatccttttaaattaaaaatgaagttttaaatcaatctaaagtata |
| | | tatgagtaaacttggtctgacagttaccaatgcttaatcagtgaggcacctatctcagcgatctg |
| | | tctatttcgttcatccatagttgcctgactcggggggggggggcgctgaggtctgcctcgtgaag |
| | | aaggtgttgctgactcataccaggcctgaatcgccccatcatccagccagaaagtgagggagcca |
| | | cggttgatgagagctttgttgtaggtggaccagttggtgattttgaacttttgctttgccacgga |
| | | acggtctgcgttgtcgggaagatgcgtgatctgatccttcaactcagcaaaagttcgatttattc |
| | | aacaaagccgccgtcccgtcaagtcagcgtaatgctctgccagtgttacaaccaattaaccaatt |
| | | ctgattagaaaaactcatcgagcatcaaatgaaactgcaatttattcatatcaggattatcaata |
| | | ccatatttttgaaaaagccgtttctgtaatgaaggagaaaactcaccgaggcagttccataggat |
| | | ggcaagatcctggtatcggtctgcgattccgactcgtccaacatcaatacaacctattaatttcc |
| | | cctcgtcaaaaataaggttatcaagtgagaaatcaccatgagtgacgactgaatccggtgagaat |
| | | ggcaaaagcttatgcatttctttccagacttgttcaacaggccagccattacgctcgtcatcaaa |
| | | atcactcgcatcaaccaaaccgttattcattcgtgattgcgcctgagcgagacgaaatacgcgat |
| | | cgctgttaaaaggacaattacaaacaggaatcgaatgcaaccggcgcaggaacactgccagcgca |
| | | tcaacaatattttcacctgaatcaggatattcttctaatacctggaatgctgttttcccggggat |
| | | cgcagtggtgagtaaccatgcatcatcaggagtacggataaaatgcttgatggtcggaagaggca |
| | | taaattccgtcagccagtttagtctgaccatctcatctgtaacatcattggcaacgctacctttg |
| | | ccatgtttcagaaacaactctggcgcatcgggcttcccatacaatcgatagattgtcgcacctga |
| | | ttgcccgacattatcgcgagcccatttatacccatataaatcagcatccatgttggaatttaatc |
| | | gcggcctcgagcaagacgtttcccgttgaatatggctcataacaccccttgtattactgtttatg |
| | | taagcagacagttttattgttcatgatgatatatttttatcttgtgcaatgtaacatcagagatt |
| | | ttgagacacaacgtggctttccccccccccccattattgaagcatttatcagggttattgtctca |
| | | tgagcggatacatatttgaatgtatttagaaaaataaacaaataggggttccgcgcacatttccc |
| | | cgaaaagtgccacctgacgtctaagaaaccattattatcatgacattaacctataaaaataggcg |
| | | tatcacgaggccctttcgtctcgcgcgtttcggtgatgacggtgaaaacctctgacacatgcagc |
| | | tcccggagacggtcacagcttgtctgtaagcggatgccgggagcagacaagcccgtcagggcgcg |
| | | tcagcgggtgttggcgggtgtcggggctggcttaactatgcggcatcagagcagattgtactgag |
| | | agtgcaccatatgcggtgtgaaataccgcacagatgcgtaaggagaaaataccgcatcagattgg |
| | | ctat |
| 26 | Doggybone DNA | gcgtataatggactattgtgtgctgatatgtacacctgaggacacaggtacaggactcagctgca |
| | plasmid expressing | ccatatgcggtgtgAAATACCGCACAGATGCGTAAGGAGAAAATACCGCATCAGATTGGCTATtg |
| | hGNE2 and | gccattgcatacgttgtatccatatcataatatgtacatttatattggctcatgtccaacattac |
| | knockdown M743T | cgccatgttgacattgattattgactagttattaatagtaatcaattacggggtcattagttcat |
| | hGNE2 mutation (bi- | agcccatatatggagttccgcgttacataacttacggtaaatggcccgcctggctgaccgcccaa |
| | shRNA is behind the | cgacccccgcccattgacgtcaataatgacgtatgttcccatagtaacgccaatagggactttcc |
| | hGNE2 mRNA unit; | attgacgtcaatgggtggagtatttacggtaaactgcccacttggcagtacatcaagtgtatcat |
| | sequence of bi-shRNA | atgccaagtacgccccctattgacgtcaatgacggtaaatggcccgcctggcattatgcccagta |
| | is in bold) | catgaccttatgggactttcctacttggcagtacatctacgtattagtcatcgctattaccatgg |
| | | tgatgcggttttggcagtacatcaatgggcgtggatagcggtttgactcacggggatttccaagt |
| | | ctccaccccattgacgtcaatgggagtttgttttggcaccaaaatcaacgggactttccaaaatg |
| | | tcgtaacaactccgccccattgacgcaaatgggcggtaggcgtgtacggtgggaggtctatataa |
| | | gcagagctcgtttagtgaaccgtcagatcgcctggagacgccatccacgctgttttgacctccat |
| | | agaagacaccgggaccgatccagcctccgcggccgggaacggtgcattggaacgcggattccccg |
| | | tgccaagagtgacgtaagtaccgcctatagactctataggcacacccctttggctcttatgcatg |
| | | ctatactgtttttggcttggggcctatacacccccgcttccttatgctataggtgatggtatagc |
| | | ttagcctataggtgtgggttattgaccattattgaccactccaacggtggagggcagtgtagtct |
| | | gagcagtactcgttgctgccgcgcgcgccaccagacataatagctgacagactaacagactgttc |
| | | ctttccatgggtcttttctgcagtcaccgtcgtcgacggtatcgataagcttgatatcgaattcg |
| | | cccttatggagaagaatggaaataaccgaaagctgcgggtttgtgttgctacttgtaaccgtgca |
| | | gattattctaaacttgccccgatcatgtttggcattaaaaccgaacctgagttctttgaacttga |
| | | tgttgtggtacttggctctcacctgatagatgactatggaaatacatatcgaatgattgaacaag |
| | | atgactttgacattaacaccaggctacacacaattgtgaggggagaagatgaggcagccatggtg |
| | | gagtcagtaggcctggccctagtgaagctgccagatgtccttaatcgcctgaagcctgatatcat |
| | | gattgttcatggagacaggtttgatgccctggctctggccacatctgctgccttgatgaacatcc |
| | | gaatccttcacattgaaggtggggaagtcagtgggaccattgatgactctatcagacatgccata |
| | | acaaaactggctcattatcatgtgtgctgcacccgcagtgcagagcagcacctgatatccatgtg |
| | | tgaggaccatgatcgcatccttttggcaggctgcccttcctatgacaaacttctctcagccaaga |
| | | acaaagactacatgagcatcattcgcatgtggctaggtgatgatgtaaaatctaaagattacatt |
| | | gttgcactacagcaccctgtgaccactgacattaagcattccataaaaatgtttgaattaacatt |
| | | ggatgcacttatctcatttaacaagcggaccctagtcctgtttccaaatattgacgcagggagca |
| | | aagagatggttcgagtgatgcggaagaagggcattgagcatcatcccaactttcgtgcagttaaa |
| | | cacgtcccatttgaccagtttatacagttggttgcccatgctggctgtatgattgggaacagcag |
| | | ctgtggggttcgagaagttggagcttttggaacacctgtgatcaacctgggaacacgtcagattg |
| | | gaagagaaacaggggagaatgttcttcatgtccgggatgctgacacccaagacaaaatattgcaa |
| | | gcactgcaccttcagtttggtaaacagtacccttgttcaaagatatatggggatggaaatgctgt |
| | | tccaaggattttgaagtttctcaaatctatcgatcttcaagagccactgcaaaagaaattctgct |
| | | ttcctcctgtgaaggagaatatctctcaagatattgaccatattcttgaaactctaagtgccttg |
| | | gccgttgatcttggcgggacgaacctccgagttgcaatagtcagcatgaagggtgaaatagttaa |
| | | gaagtatactcagttcaatcctaaaacctatgaagagaggattaatttaatcctacagatgtgtg |
| | | tggaagctgcagcagaagctgtaaaactgaactgcagaattttgggagtaggcatttccacaggt |
| | | ggccgtgtaaatcctcgggaaggaattgtgctgcattcaaccaaactgatccaagagtggaactc |
| | | tgtggaccttaggacccccctttctgacactttgcatctccctgtgtgggtagacaatgatggca |
| | | actgtgctgccctggcggaaaggaaatttggccaaggaaagggactggaaaactttgttacactt |
| | | atcacaggcacaggaatcggtggtggaattatccatcagcatgaattgatccacggaagctcctt |
| | | ctgtgctgcagaactgggccaccttgttgtgtctctggatgggcctgattgttcctgtggaagcc |
| | | atgggtgcattgaagcatacgcctctggaatggccttgcagagggaggcaaaaaagctccatgat |
| | | gaggacctgctcttggtggaagggatgtcagtgccaaaagatgaggctgtgggtgcgctccatct |
| | | catccaagctgcgaaacttggcaatgcgaaggcccagagcatcctaagaacagctggaacagctt |
| | | tgggtcttggggttgtgaacatcctccataccatgaatccctcccttgtgatcctctccggagtc |
| | | ctggccagtcactatatccacattgtcaaagacgtcattcgccagcaggccttgtcctccgtgca |
| | | ggacgtggatgtggtggtttcggatttggttgaccccgccctgctgggtgctgccagcatggttc |
| | | tggactacacaacacgcaggatctactagaagggcgaattcacgtgggcccggtaccgtatactc |
| | | tagagcggccgcggtttcttccccattagggattatgctgaatttgtatggtttatagttgttag |
| | | agtttgaggtgttaattctaattatctatttcaaatttagcaggaaaaaagagaacatcaccttg |
| | | taaaactgaagattgtgaccagtcagaataatgtctgctgggtgctgccagcacggtagatatgt |
| | | gcatctaccgtgctggcagcacccagcagcattatggtgacagctgcctcgggaagccaagttgg |
| | | gctttaaagtgcagggcctgctgatgttgagtgctttttgttcatactgtgtgcgaccatcatcg |
| | | tagtgaagtagattagcatctaccgtgctggcagcacccagcagcataagaagttatgtattcat |
| | | ccaataattcaagccaagcaagtatataggtgttttaatagtttttgttttggggattgtgacca |
| | | gaagattttgaaaattaaatattactgaagatttcgacttccactgttaaatgtacaagatacat |
| | | gaaatattaaagaaaatgtgtaactttttgtgtaaatacatcttgtatccagatctttttccctc |
| | | tgccaaaaattatggggacatcatgaagccccttgagcatctgacttctggctaataaaggaaat |
| | | ttattttcattgcaatagtgtgttggaattttttgtgtctctcactcggaaggacatatgggagg |
| | | gcaaatcatttaaaacatcagaatgagtatttggtttagagtttggcaacatatgcccattcttc |
| | | cgcttcctcgctcactgactcgctgcgctcggtcgttcggctgcggcgagcggtatcagctcact |
| | | caaaggcggtaatacggttatccacagaatcaggggataacgcaggaaagaacatggaggtgaga |
| | | ttgtgttcggcatgcctcacaggcagatctatcagcacacaattgcccattatacgc |
| 27 | mRNA transcript | ATGGAAACCTATGGTTATCTGCAGAGGGAGTCATGCTTTCAAGGACCTCATGAACTCTATTTTAA |
| | encoding wild-type | GAACCTCTCAAAACGAAACAAGCAAATCATGGAGAAGAATGGAAATAACCGAAAGCTGCGGGTTT |
| | hGNE2 | GTGTTGCTACTTGTAACCGTGCAGATTATTCTAAACTTGCCCCGATCATGTTTGGCATTAAAACC |
| | | GAACCTGAGTTCTTTGAACTTGATGTTGTGGTACTTGGCTCTCACCTGATAGATGACTATGGAAA |
| | | TACATATCGAATGATTGAACAAGATGACTTTGACATTAACACCAGGCTACACACAATTGTGAGGG |
| | | GAGAAGATGAGGCAGCCATGGTGGAGTCAGTAGGCCTGGCCCTAGTGAAGCTGCCAGATGTCCTT |
| | | AATCGCCTGAAGCCTGATATCATGATTGTTCATGGAGACAGGTTTGATGCCCTGGCTCTGGCCAC |
| | | ATCTGCTGCCTTGATGAACATCCGAATCCTTCACATTGAAGGTGGGGAAGTCAGTGGGACCATTG |
| | | ATGACTCTATCAGACATGCCATAACAAAACTGGCTCATTATCATGTGTGCTGCACCCGCAGTGCA |
| | | GAGCAGCACCTGATATCCATGTGTGAGGACCATGATCGCATCCTTTTGGCAGGCTGCCCTTCCTA |
| | | TGACAAACTTCTCTCAGCCAAGAACAAAGACTACATGAGCATCATTCGCATGTGGCTAGGTGATG |
| | | ATGTAAAATCTAAAGATTACATTGTTGCACTACAGCACCCTGTGACCACTGACATTAAGCATTCC |
| | | ATAAAAATGTTTGAATTAACATTGGATGCACTTATCTCATTTAACAAGCGGACCCTAGTCCTGTT |
| | | TCCAAATATTGACGCAGGGAGCAAAGAGATGGTTCGAGTGATGCGGAAGAAGGGCATTGAGCATC |
| | | ATCCCAACTTTCGTGCAGTTAAACACGTCCCATTTGACCAGTTTATACAGTTGGTTGCCCATGCT |
| | | GGCTGTATGATTGGGAACAGCAGCTGTGGGGTTCGAGAAGTTGGAGCTTTTGGAACACCTGTGAT |
| | | CAACCTGGGAACACGTCAGATTGGAAGAGAAACAGGGGAGAATGTTCTTCATGTCCGGGATGCTG |
| | | ACACCCAAGACAAAATATTGCAAGCACTGCACCTTCAGTTTGGTAAACAGTACCCTTGTTCAAAG |
| | | ATATATGGGGATGGAAATGCTGTTCCAAGGATTTTGAAGTTTCTCAAATCTATCGATCTTCAAGA |
| | | GCCACTGCAAAAGAAATTCTGCTTTCCTCCTGTGAAGGAGAATATCTCTCAAGATATTGACCATA |
| | | TTCTTGAAACTCTAAGTGCCTTGGCCGTTGATCTTGGCGGGACGAACCTCCGAGTTGCAATAGTC |
| | | AGCATGAAGGGTGAAATAGTTAAGAAGTATACTCAGTTCAATCCTAAAACCTATGAAGAGAGGAT |
| | | TAATTTAATCCTACAGATGTGTGTGGAAGCTGCAGCAGAAGCTGTAAAACTGAACTGCAGAATTT |
| | | TGGGAGTAGGCATTTCCACAGGTGGCCGTGTAAATCCTCGGGAAGGAATTGTGCTGCATTCAACC |
| | | AAACTGATCCAAGAGTGGAACTCTGTGGACCTTAGGACCCCCCTTTCTGACACTTTGCATCTCCC |
| | | TGTGTGGGTAGACAATGATGGCAACTGTGCTGCCCTGGCGGAAAGGAAATTTGGCCAAGGAAAGG |
| | | GACTGGAAAACTTTGTTACACTTATCACAGGCACAGGAATCGGTGGTGGAATTATCCATCAGCAT |
| | | GAATTGATCCACGGAAGCTCCTTCTGTGCTGCAGAACTGGGCCACCTTGTTGTGTCTCTGGATGG |
| | | GCCTGATTGTTCCTGTGGAAGCCATGGGTGCATTGAAGCATACGCCTCTGGAATGGCCTTGCAGA |
| | | GGGAGGCAAAAAAGCTCCATGATGAGGACCTGCTCTTGGTGGAAGGGATGTCAGTGCCAAAAGAT |
| | | GAGGCTGTGGGTGCGCTCCATCTCATCCAAGCTGCGAAACTTGGCAATGCGAAGGCCCAGAGCAT |
| | | CCTAAGAACAGCTGGAACAGCTTTGGGTCTTGGGGTTGTGAACATCCTCCATACCATGAATCCCT |
| | | CCCTTGTGATCCTCTCCGGAGTCCTGGCCAGTCACTATATCCACATTGTCAAAGACGTCATTCGC |
| | | CAGCAGGCCTTGTCCTCCGTGCAGGACGTGGATGTGGTGGTTTCGGATTTGGTTGACCCCGCCCT |
| | | GCTGGGTGCTGCCAGCATGGTTCTGGACTACACAACACGCAGGATCTACTAG |
| 28 | Protein sequence of | METYGYLQRESCFQGPHELYFKNLSKRNKQIMEKNGNNRKLRVCVATCNRADYSKLAPIMFGIKT |
| | hGNE2 (D207, V603, | EPEFFELDVVVLGSHLIDDYGNTYRMIEQDDFDINTRLHTIVRGEDEAAMVESVGLALVKLPDVL |
| | and M743 are in bold) | NRLKPDIMIVHGDRFDALALATSAALMNIRILHIEGGEVSGTIDDSIRHAITKLAHYHVCCTRSA |
| | | EQHLISMCEDHDRILLAGCPSYDKLLSAKNKDYMSIIRMWLGDDVKSKDYIVALQHPVTTDIKHS |
| | | IKMFELTLDALISFNKRTLVLFPNIDAGSKEMVRVMRKKGIEHHPNFRAVKHVPFDQFIQLVAHA |
| | | GCMIGNSSCGVREVGAFGTPVINLGTRQIGRETGENVLHVRDADTQDKILQALHLQFGKQYPCSK |
| | | IYGDGNAVPRILKFLKSIDLQEPLQKKFCFPPVKENISQDIDHILETLSALAVDLGGTNLRVAIV |
| | | SMKGEIVKKYTQFNPKTYEERINLILQMCVEAAAEAVKLNCRILGVGISTGGRVNPREGIVLHST |
| | | KLIQEWNSVDLRTPLSDTLHLPVWVDNDGNCAALAERKFGQGKGLENFVTLITGTGIGGGIIHQH |
| | | ELIHGSSFCAAELGHLVVSLDGPDCSCGSHGCIEAYASGMALQREAKKLHDEDLLLVEGMSVPKD |
| | | EAVGALHLIQAAKLGNAKAQSILRTAGTALGLGVVNILHTMNPSLVILSGVLASHYIHIVKDVIR |
| | | QQALSSVQDVDVVVSDLVDPALLGAASMVLDYTTRRIY |
| 29 | Vector expressing | TGGCCATTGCATACGTTGTATCCATATCATAATATGTACATTTATATTGGCTCATGTCCAACATT |
| | hGNE2 in pUMVC3 | ACCGCCATGTTGACATTGATTATTGACTAGTTATTAATAGTAATCAATTACGGGGTCATTAGTTC |
| | backbone | ATAGCCCATATATGGAGTTCCGCGTTACATAACTTACGGTAAATGGCCCGCCTGGCTGACCGCCC |
| | | AACGACCCCCGCCCATTGACGTCAATAATGACGTATGTTCCCATAGTAACGCCAATAGGGACTTT |
| | | CCATTGACGTCAATGGGTGGAGTATTTACGGTAAACTGCCCACTTGGCAGTACATCAAGTGTATC |
| | | ATATGCCAAGTACGCCCCCTATTGACGTCAATGACGGTAAATGGCCCGCCTGGCATTATGCCCAG |
| | | TACATGACCTTATGGGACTTTCCTACTTGGCAGTACATCTACGTATTAGTCATCGCTATTACCAT |
| | | GGTGATGCGGTTTTGGCAGTACATCAATGGGCGTGGATAGCGGTTTGACTCACGGGGATTTCCAA |
| | | GTCTCCACCCCATTGACGTCAATGGGAGTTTGTTTTGGCACCAAAATCAACGGGACTTTCCAAAA |
| | | TGTCGTAACAACTCCGCCCCATTGACGCAAATGGGCGGTAGGCGTGTACGGTGGGAGGTCTATAT |
| | | AAGCAGAGCTCGTTTAGTGAACCGTCAGATCGCCTGGAGACGCCATCCACGCTGTTTTGACCTCC |
| | | ATAGAAGACACCGGGACCGATCCAGCCTCCGCGGCCGGGAACGGTGCATTGGAACGCGGATTCCC |
| | | CGTGCCAAGAGTGACGTAAGTACCGCCTATAGACTCTATAGGCACACCCCTTTGGCTCTTATGCA |
| | | TGCTATACTGTTTTTGGCTTGGGGCCTATACACCCCCGCTTCCTTATGCTATAGGTGATGGTATA |
| | | GCTTAGCCTATAGGTGTGGGTTATTGACCATTATTGACCACTCCAACGGTGGAGGGCAGTGTAGT |
| | | CTGAGCAGTACTCGTTGCTGCCGCGCGCGCCACCAGACATAATAGCTGACAGACTAACAGACTGT |
| | | TCCTTTCCATGGGTCTTTTCTGCAGTCACCGTCGTCGACGGTATCGATAAGCTTGATATCGAATT |
| | | CGCCCTTATGGAGAAGAATGGAAATAACCGAAAGCTGCGGGTTTGTGTTGCTACTTGTAACCGTG |
| | | CAGATTATTCTAAACTTGCCCCGATCATGTTTGGCATTAAAACCGAACCTGAGTTCTTTGAACTT |
| | | GATGTTGTGGTACTTGGCTCTCACCTGATAGATGACTATGGAAATACATATCGAATGATTGAACA |
| | | AGATGACTTTGACATTAACACCAGGCTACACACAATTGTGAGGGGAGAAGATGAGGCAGCCATGG |
| | | TGGAGTCAGTAGGCCTGGCCCTAGTGAAGCTGCCAGATGTCCTTAATCGCCTGAAGCCTGATATC |
| | | ATGATTGTTCATGGAGACAGGTTTGATGCCCTGGCTCTGGCCACATCTGCTGCCTTGATGAACAT |
| | | CCGAATCCTTCACATTGAAGGTGGGGAAGTCAGTGGGACCATTGATGACTCTATCAGACATGCCA |
| | | TAACAAAACTGGCTCATTATCATGTGTGCTGCACCCGCAGTGCAGAGCAGCACCTGATATCCATG |
| | | TGTGAGGACCATGATCGCATCCTTTTGGCAGGCTGCCCTTCCTATGACAAACTTCTCTCAGCCAA |
| | | GAACAAAGACTACATGAGCATCATTCGCATGTGGCTAGGTGATGATGTAAAATCTAAAGATTACA |
| | | TTGTTGCACTACAGCACCCTGTGACCACTGACATTAAGCATTCCATAAAAATGTTTGAATTAACA |
| | | TTGGATGCACTTATCTCATTTAACAAGCGGACCCTAGTCCTGTTTCCAAATATTGACGCAGGGAG |
| | | CAAAGAGATGGTTCGAGTGATGCGGAAGAAGGGCATTGAGCATCATCCCAACTTTCGTGCAGTTA |
| | | AACACGTCCCATTTGACCAGTTTATACAGTTGGTTGCCCATGCTGGCTGTATGATTGGGAACAGC |
| | | AGCTGTGGGGTTCGAGAAGTTGGAGCTTTTGGAACACCTGTGATCAACCTGGGAACACGTCAGAT |
| | | TGGAAGAGAAACAGGGGAGAATGTTCTTCATGTCCGGGATGCTGACACCCAAGACAAAATATTGC |
| | | AAGCACTGCACCTTCAGTTTGGTAAACAGTACCCTTGTTCAAAGATATATGGGGATGGAAATGCT |
| | | GTTCCAAGGATTTTGAAGTTTCTCAAATCTATCGATCTTCAAGAGCCACTGCAAAAGAAATTCTG |
| | | CTTTCCTCCTGTGAAGGAGAATATCTCTCAAGATATTGACCATATTCTTGAAACTCTAAGTGCCT |
| | | TGGCCGTTGATCTTGGCGGGACGAACCTCCGAGTTGCAATAGTCAGCATGAAGGGTGAAATAGTT |
| | | AAGAAGTATACTCAGTTCAATCCTAAAACCTATGAAGAGAGGATTAATTTAATCCTACAGATGTG |
| | | TGTGGAAGCTGCAGCAGAAGCTGTAAAACTGAACTGCAGAATTTTGGGAGTAGGCATTTCCACAG |
| | | GTGGCCGTGTAAATCCTCGGGAAGGAATTGTGCTGCATTCAACCAAACTGATCCAAGAGTGGAAC |
| | | TCTGTGGACCTTAGGACCCCCCTTTCTGACACTTTGCATCTCCCTGTGTGGGTAGACAATGATGG |
| | | CAACTGTGCTGCCCTGGCGGAAAGGAAATTTGGCCAAGGAAAGGGACTGGAAAACTTTGTTACAC |
| | | TTATCACAGGCACAGGAATCGGTGGTGGAATTATCCATCAGCATGAATTGATCCACGGAAGCTCC |
| | | TTCTGTGCTGCAGAACTGGGCCACCTTGTTGTGTCTCTGGATGGGCCTGATTGTTCCTGTGGAAG |
| | | CCATGGGTGCATTGAAGCATACGCCTCTGGAATGGCCTTGCAGAGGGAGGCAAAAAAGCTCCATG |
| | | ATGAGGACCTGCTCTTGGTGGAAGGGATGTCAGTGCCAAAAGATGAGGCTGTGGGTGCGCTCCAT |
| | | CTCATCCAAGCTGCGAAACTTGGCAATGCGAAGGCCCAGAGCATCCTAAGAACAGCTGGAACAGC |
| | | TTTGGGTCTTGGGGTTGTGAACATCCTCCATACCATGAATCCCTCCCTTGTGATCCTCTCCGGAG |
| | | TCCTGGCCAGTCACTATATCCACATTGTCAAAGACGTCATTCGCCAGCAGGCCTTGTCCTCCGTG |
| | | CAGGACGTGGATGTGGTGGTTTCGGATTTGGTTGACCCCGCCCTGCTGGGTGCTGCCAGCATGGT |
| | | TCTGGACTACACAACACGCAGGATCTACTAGAAGGGCGAATTCACGTGGGCCCGGTACCGTATAC |
| | | TCTAGAGCGGCCGCGGATCCAGATCTTTTTCCCTCTGCCAAAAATTATGGGGACATCATGAAGCC |
| | | CCTTGAGCATCTGACTTCTGGCTAATAAAGGAAATTTATTTTCATTGCAATAGTGTGTTGGAATT |
| | | TTTTGTGTCTCTCACTCGGAAGGACATATGGGAGGGCAAATCATTTAAAACATCAGAATGAGTAT |
| | | TTGGTTTAGAGTTTGGCAACATATGCCCATTCTTCCGCTTCCTCGCTCACTGACTCGCTGCGCTC |
| | | GGTCGTTCGGCTGCGGCGAGCGGTATCAGCTCACTCAAAGGCGGTAATACGGTTATCCACAGAAT |
| | | CAGGGGATAACGCAGGAAAGAACATGTGAGCAAAAGGCCAGCAAAAGGCCAGGAACCGTAAAAAG |
| | | GCCGCGTTGCTGGCGTTTTTCCATAGGCTCCGCCCCCCTGACGAGCATCACAAAAATCGACGCTC |
| | | AAGTCAGAGGTGGCGAAACCCGACAGGACTATAAAGATACCAGGCGTTTCCCCCTGGAAGCTCCC |
| | | TCGTGCGCTCTCCTGTTCCGACCCTGCCGCTTACCGGATACCTGTCCGCCTTTCTCCCTTCGGGA |
| | | AGCGTGGCGCTTTCTCATAGCTCACGCTGTAGGTATCTCAGTTCGGTGTAGGTCGTTCGCTCCAA |
| | | GCTGGGCTGTGTGCACGAACCCCCCGTTCAGCCCGACCGCTGCGCCTTATCCGGTAACTATCGTC |
| | | TTGAGTCCAACCCGGTAAGACACGACTTATCGCCACTGGCAGCAGCCACTGGTAACAGGATTAGC |
| | | AGAGCGAGGTATGTAGGCGGTGCTACAGAGTTCTTGAAGTGGTGGCCTAACTACGGCTACACTAG |
| | | AAGAACAGTATTTGGTATCTGCGCTCTGCTGAAGCCAGTTACCTTCGGAAAAAGAGTTGGTAGCT |
| | | CTTGATCCGGCAAACAAACCACCGCTGGTAGCGGTGGTTTTTTTGTTTGCAAGCAGCAGATTACG |
| | | CGCAGAAAAAAAGGATCTCAAGAAGATCCTTTGATCTTTTCTACGGGGTCTGACGCTCAGTGGAA |
| | | CGAAAACTCACGTTAAGGGATTTTGGTCATGAGATTATCAAAAAGGATCTTCACCTAGATCCTTT |
| | | TAAATTAAAAATGAAGTTTTAAATCAATCTAAAGTATATATGAGTAAACTTGGTCTGACAGTTAC |
| | | CAATGCTTAATCAGTGAGGCACCTATCTCAGCGATCTGTCTATTTCGTTCATCCATAGTTGCCTG |
| | | ACTCGGGGGGGGGGGGCGCTGAGGTCTGCCTCGTGAAGAAGGTGTTGCTGACTCATACCAGGCCT |
| | | GAATCGCCCCATCATCCAGCCAGAAAGTGAGGGAGCCACGGTTGATGAGAGCTTTGTTGTAGGTG |
| | | GACCAGTTGGTGATTTTGAACTTTTGCTTTGCCACGGAACGGTCTGCGTTGTCGGGAAGATGCGT |
| | | GATCTGATCCTTCAACTCAGCAAAAGTTCGATTTATTCAACAAAGCCGCCGTCCCGTCAAGTCAG |
| | | CGTAATGCTCTGCCAGTGTTACAACCAATTAACCAATTCTGATTAGAAAAACTCATCGAGCATCA |
| | | AATGAAACTGCAATTTATTCATATCAGGATTATCAATACCATATTTTTGAAAAAGCCGTTTCTGT |
| | | AATGAAGGAGAAAACTCACCGAGGCAGTTCCATAGGATGGCAAGATCCTGGTATCGGTCTGCGAT |
| | | TCCGACTCGTCCAACATCAATACAACCTATTAATTTCCCCTCGTCAAAAATAAGGTTATCAAGTG |
| | | AGAAATCACCATGAGTGACGACTGAATCCGGTGAGAATGGCAAAAGCTTATGCATTTCTTTCCAG |
| | | ACTTGTTCAACAGGCCAGCCATTACGCTCGTCATCAAAATCACTCGCATCAACCAAACCGTTATT |
| | | CATTCGTGATTGCGCCTGAGCGAGACGAAATACGCGATCGCTGTTAAAAGGACAATTACAAACAG |
| | | GAATCGAATGCAACCGGCGCAGGAACACTGCCAGCGCATCAACAATATTTTCACCTGAATCAGGA |
| | | TATTCTTCTAATACCTGGAATGCTGTTTTCCCGGGGATCGCAGTGGTGAGTAACCATGCATCATC |
| | | AGGAGTACGGATAAAATGCTTGATGGTCGGAAGAGGCATAAATTCCGTCAGCCAGTTTAGTCTGA |
| | | CCATCTCATCTGTAACATCATTGGCAACGCTACCTTTGCCATGTTTCAGAAACAACTCTGGCGCA |
| | | TCGGGCTTCCCATACAATCGATAGATTGTCGCACCTGATTGCCCGACATTATCGCGAGCCCATTT |
| | | ATACCCATATAAATCAGCATCCATGTTGGAATTTAATCGCGGCCTCGAGCAAGACGTTTCCCGTT |
| | | GAATATGGCTCATAACACCCCTTGTATTACTGTTTATGTAAGCAGACAGTTTTATTGTTCATGAT |
| | | GATATATTTTTATCTTGTGCAATGTAACATCAGAGATTTTGAGACACAACGTGGCTTTCCCCCCC |
| | | CCCCCATTATTGAAGCATTTATCAGGGTTATTGTCTCATGAGCGGATACATATTTGAATGTATTT |
| | | AGAAAAATAAACAAATAGGGGTTCCGCGCACATTTCCCCGAAAAGTGCCACCTGACGTCTAAGAA |
| | | ACCATTATTATCATGACATTAACCTATAAAAATAGGCGTATCACGAGGCCCTTTCGTCTCGCGCG |
| | | TTTCGGTGATGACGGTGAAAACCTCTGACACATGCAGCTCCCGGAGACGGTCACAGCTTGTCTGT |
| | | AAGCGGATGCCGGGAGCAGACAAGCCCGTCAGGGCGCGTCAGCGGGTGTTGGCGGGTGTCGGGGC |
| | | TGGCTTAACTATGCGGCATCAGAGCAGATTGTACTGAGAGTGCACCATATGCGGTGTGAAATACC |
| | | GCACAGATGCGTAAGGAGAAAATACCGCATCAGATTGGCTAT |
| 30 | Vector expressing | TGGCCATTGCATACGTTGTATCCATATCATAATATGTACATTTATATTGGCTCATGTCCAACATT |
| | mutant hGNE2 M743T | ACCGCCATGTTGACATTGATTATTGACTAGTTATTAATAGTAATCAATTACGGGGTCATTAGTTC |
| | in pUMVC3 backbone | ATAGCCCATATATGGAGTTCCGCGTTACATAACTTACGGTAAATGGCCCGCCTGGCTGACCGCCC |
| | | AACGACCCCCGCCCATTGACGTCAATAATGACGTATGTTCCCATAGTAACGCCAATAGGGACTTT |
| | | CCATTGACGTCAATGGGTGGAGTATTTACGGTAAACTGCCCACTTGGCAGTACATCAAGTGTATC |
| | | ATATGCCAAGTACGCCCCCTATTGACGTCAATGACGGTAAATGGCCCGCCTGGCATTATGCCCAG |
| | | TACATGACCTTATGGGACTTTCCTACTTGGCAGTACATCTACGTATTAGTCATCGCTATTACCAT |
| | | GGTGATGCGGTTTTGGCAGTACATCAATGGGCGTGGATAGCGGTTTGACTCACGGGGATTTCCAA |
| | | GTCTCCACCCCATTGACGTCAATGGGAGTTTGTTTTGGCACCAAAATCAACGGGACTTTCCAAAA |
| | | TGTCGTAACAACTCCGCCCCATTGACGCAAATGGGCGGTAGGCGTGTACGGTGGGAGGTCTATAT |
| | | AAGCAGAGCTCGTTTAGTGAACCGTCAGATCGCCTGGAGACGCCATCCACGCTGTTTTGACCTCC |
| | | ATAGAAGACACCGGGACCGATCCAGCCTCCGCGGCCGGGAACGGTGCATTGGAACGCGGATTCCC |
| | | CGTGCCAAGAGTGACGTAAGTACCGCCTATAGACTCTATAGGCACACCCCTTTGGCTCTTATGCA |
| | | TGCTATACTGTTTTTGGCTTGGGGCCTATACACCCCCGCTTCCTTATGCTATAGGTGATGGTATA |
| | | GCTTAGCCTATAGGTGTGGGTTATTGACCATTATTGACCACTCCAACGGTGGAGGGCAGTGTAGT |
| | | CTGAGCAGTACTCGTTGCTGCCGCGCGCGCCACCAGACATAATAGCTGACAGACTAACAGACTGT |
| | | TCCTTTCCATGGGTCTTTTCTGCAGTCACCGTCGTCGACGGTATCGATAAGCTTGATATCGAATT |
| | | CGCCCTTATGGAGAAGAATGGAAATAACCGAAAGCTGCGGGTTTGTGTTGCTACTTGTAACCGTG |
| | | CAGATTATTCTAAACTTGCCCCGATCATGTTTGGCATTAAAACCGAACCTGAGTTCTTTGAACTT |
| | | GATGTTGTGGTACTTGGCTCTCACCTGATAGATGACTATGGAAATACATATCGAATGATTGAACA |
| | | AGATGACTTTGACATTAACACCAGGCTACACACAATTGTGAGGGGAGAAGATGAGGCAGCCATGG |
| | | TGGAGTCAGTAGGCCTGGCCCTAGTGAAGCTGCCAGATGTCCTTAATCGCCTGAAGCCTGATATC |
| | | ATGATTGTTCATGGAGACAGGTTTGATGCCCTGGCTCTGGCCACATCTGCTGCCTTGATGAACAT |
| | | CCGAATCCTTCACATTGAAGGTGGGGAAGTCAGTGGGACCATTGATGACTCTATCAGACATGCCA |
| | | TAACAAAACTGGCTCATTATCATGTGTGCTGCACCCGCAGTGCAGAGCAGCACCTGATATCCATG |
| | | TGTGAGGACCATGATCGCATCCTTTTGGCAGGCTGCCCTTCCTATGACAAACTTCTCTCAGCCAA |
| | | GAACAAAGACTACATGAGCATCATTCGCATGTGGCTAGGTGATGATGTAAAATCTAAAGATTACA |
| | | TTGTTGCACTACAGCACCCTGTGACCACTGACATTAAGCATTCCATAAAAATGTTTGAATTAACA |
| | | TTGGATGCACTTATCTCATTTAACAAGCGGACCCTAGTCCTGTTTCCAAATATTGACGCAGGGAG |
| | | CAAAGAGATGGTTCGAGTGATGCGGAAGAAGGGCATTGAGCATCATCCCAACTTTCGTGCAGTTA |
| | | AACACGTCCCATTTGACCAGTTTATACAGTTGGTTGCCCATGCTGGCTGTATGATTGGGAACAGC |
| | | AGCTGTGGGGTTCGAGAAGTTGGAGCTTTTGGAACACCTGTGATCAACCTGGGAACACGTCAGAT |
| | | TGGAAGAGAAACAGGGGAGAATGTTCTTCATGTCCGGGATGCTGACACCCAAGACAAAATATTGC |
| | | AAGCACTGCACCTTCAGTTTGGTAAACAGTACCCTTGTTCAAAGATATATGGGGATGGAAATGCT |
| | | GTTCCAAGGATTTTGAAGTTTCTCAAATCTATCGATCTTCAAGAGCCACTGCAAAAGAAATTCTG |
| | | CTTTCCTCCTGTGAAGGAGAATATCTCTCAAGATATTGACCATATTCTTGAAACTCTAAGTGCCT |
| | | TGGCCGTTGATCTTGGCGGGACGAACCTCCGAGTTGCAATAGTCAGCATGAAGGGTGAAATAGTT |
| | | AAGAAGTATACTCAGTTCAATCCTAAAACCTATGAAGAGAGGATTAATTTAATCCTACAGATGTG |
| | | TGTGGAAGCTGCAGCAGAAGCTGTAAAACTGAACTGCAGAATTTTGGGAGTAGGCATTTCCACAG |
| | | GTGGCCGTGTAAATCCTCGGGAAGGAATTGTGCTGCATTCAACCAAACTGATCCAAGAGTGGAAC |
| | | TCTGTGGACCTTAGGACCCCCCTTTCTGACACTTTGCATCTCCCTGTGTGGGTAGACAATGATGG |
| | | CAACTGTGCTGCCCTGGCGGAAAGGAAATTTGGCCAAGGAAAGGGACTGGAAAACTTTGTTACAC |
| | | TTATCACAGGCACAGGAATCGGTGGTGGAATTATCCATCAGCATGAATTGATCCACGGAAGCTCC |
| | | TTCTGTGCTGCAGAACTGGGCCACCTTGTTGTGTCTCTGGATGGGCCTGATTGTTCCTGTGGAAG |
| | | CCATGGGTGCATTGAAGCATACGCCTCTGGAATGGCCTTGCAGAGGGAGGCAAAAAAGCTCCATG |
| | | ATGAGGACCTGCTCTTGGTGGAAGGGATGTCAGTGCCAAAAGATGAGGCTGTGGGTGCGCTCCAT |
| | | CTCATCCAAGCTGCGAAACTTGGCAATGCGAAGGCCCAGAGCATCCTAAGAACAGCTGGAACAGC |
| | | TTTGGGTCTTGGGGTTGTGAACATCCTCCATACCATGAATCCCTCCCTTGTGATCCTCTCCGGAG |
| | | TCCTGGCCAGTCACTATATCCACATTGTCAAAGACGTCATTCGCCAGCAGGCCTTGTCCTCCGTG |
| | | CAGGACGTGGATGTGGTGGTTTCGGATTTGGTTGACCCCGCCCTGCTGGGTGCTGCCAGCACGGT |
| | | TCTGGACTACACAACACGCAGGATCTACTAGAAGGGCGAATTCACGTGGGCCCGGTACCGTATAC |
| | | TCTAGAGCGGCCGCGGATCCAGATCTTTTTCCCTCTGCCAAAAATTATGGGGACATCATGAAGCC |
| | | CCTTGAGCATCTGACTTCTGGCTAATAAAGGAAATTTATTTTCATTGCAATAGTGTGTTGGAATT |
| | | TTTTGTGTCTCTCACTCGGAAGGACATATGGGAGGGCAAATCATTTAAAACATCAGAATGAGTAT |
| | | TTGGTTTAGAGTTTGGCAACATATGCCCATTCTTCCGCTTCCTCGCTCACTGACTCGCTGCGCTC |
| | | GGTCGTTCGGCTGCGGCGAGCGGTATCAGCTCACTCAAAGGCGGTAATACGGTTATCCACAGAAT |
| | | CAGGGGATAACGCAGGAAAGAACATGTGAGCAAAAGGCCAGCAAAAGGCCAGGAACCGTAAAAAG |
| | | GCCGCGTTGCTGGCGTTTTTCCATAGGCTCCGCCCCCCTGACGAGCATCACAAAAATCGACGCTC |
| | | AAGTCAGAGGTGGCGAAACCCGACAGGACTATAAAGATACCAGGCGTTTCCCCCTGGAAGCTCCC |
| | | TCGTGCGCTCTCCTGTTCCGACCCTGCCGCTTACCGGATACCTGTCCGCCTTTCTCCCTTCGGGA |
| | | AGCGTGGCGCTTTCTCATAGCTCACGCTGTAGGTATCTCAGTTCGGTGTAGGTCGTTCGCTCCAA |
| | | GCTGGGCTGTGTGCACGAACCCCCCGTTCAGCCCGACCGCTGCGCCTTATCCGGTAACTATCGTC |
| | | TTGAGTCCAACCCGGTAAGACACGACTTATCGCCACTGGCAGCAGCCACTGGTAACAGGATTAGC |
| | | AGAGCGAGGTATGTAGGCGGTGCTACAGAGTTCTTGAAGTGGTGGCCTAACTACGGCTACACTAG |
| | | AAGAACAGTATTTGGTATCTGCGCTCTGCTGAAGCCAGTTACCTTCGGAAAAAGAGTTGGTAGCT |
| | | CTTGATCCGGCAAACAAACCACCGCTGGTAGCGGTGGTTTTTTTGTTTGCAAGCAGCAGATTACG |
| | | CGCAGAAAAAAAGGATCTCAAGAAGATCCTTTGATCTTTTCTACGGGGTCTGACGCTCAGTGGAA |
| | | CGAAAACTCACGTTAAGGGATTTTGGTCATGAGATTATCAAAAAGGATCTTCACCTAGATCCTTT |
| | | TAAATTAAAAATGAAGTTTTAAATCAATCTAAAGTATATATGAGTAAACTTGGTCTGACAGTTAC |
| | | CAATGCTTAATCAGTGAGGCACCTATCTCAGCGATCTGTCTATTTCGTTCATCCATAGTTGCCTG |
| | | ACTCGGGGGGGGGGGGCGCTGAGGTCTGCCTCGTGAAGAAGGTGTTGCTGACTCATACCAGGCCT |
| | | GAATCGCCCCATCATCCAGCCAGAAAGTGAGGGAGCCACGGTTGATGAGAGCTTTGTTGTAGGTG |
| | | GACCAGTTGGTGATTTTGAACTTTTGCTTTGCCACGGAACGGTCTGCGTTGTCGGGAAGATGCGT |
| | | GATCTGATCCTTCAACTCAGCAAAAGTTCGATTTATTCAACAAAGCCGCCGTCCCGTCAAGTCAG |
| | | CGTAATGCTCTGCCAGTGTTACAACCAATTAACCAATTCTGATTAGAAAAACTCATCGAGCATCA |
| | | AATGAAACTGCAATTTATTCATATCAGGATTATCAATACCATATTTTTGAAAAAGCCGTTTCTGT |
| | | AATGAAGGAGAAAACTCACCGAGGCAGTTCCATAGGATGGCAAGATCCTGGTATCGGTCTGCGAT |
| | | TCCGACTCGTCCAACATCAATACAACCTATTAATTTCCCCTCGTCAAAAATAAGGTTATCAAGTG |
| | | AGAAATCACCATGAGTGACGACTGAATCCGGTGAGAATGGCAAAAGCTTATGCATTTCTTTCCAG |
| | | ACTTGTTCAACAGGCCAGCCATTACGCTCGTCATCAAAATCACTCGCATCAACCAAACCGTTATT |
| | | CATTCGTGATTGCGCCTGAGCGAGACGAAATACGCGATCGCTGTTAAAAGGACAATTACAAACAG |
| | | GAATCGAATGCAACCGGCGCAGGAACACTGCCAGCGCATCAACAATATTTTCACCTGAATCAGGA |
| | | TATTCTTCTAATACCTGGAATGCTGTTTTCCCGGGGATCGCAGTGGTGAGTAACCATGCATCATC |
| | | AGGAGTACGGATAAAATGCTTGATGGTCGGAAGAGGCATAAATTCCGTCAGCCAGTTTAGTCTGA |
| | | CCATCTCATCTGTAACATCATTGGCAACGCTACCTTTGCCATGTTTCAGAAACAACTCTGGCGCA |
| | | TCGGGCTTCCCATACAATCGATAGATTGTCGCACCTGATTGCCCGACATTATCGCGAGCCCATTT |
| | | ATACCCATATAAATCAGCATCCATGTTGGAATTTAATCGCGGCCTCGAGCAAGACGTTTCCCGTT |
| | | GAATATGGCTCATAACACCCCTTGTATTACTGTTTATGTAAGCAGACAGTTTTATTGTTCATGAT |
| | | GATATATTTTTATCTTGTGCAATGTAACATCAGAGATTTTGAGACACAACGTGGCTTTCCCCCCC |
| | | CCCCCATTATTGAAGCATTTATCAGGGTTATTGTCTCATGAGCGGATACATATTTGAATGTATTT |
| | | AGAAAAATAAACAAATAGGGGTTCCGCGCACATTTCCCCGAAAAGTGCCACCTGACGTCTAAGAA |
| | | ACCATTATTATCATGACATTAACCTATAAAAATAGGCGTATCACGAGGCCCTTTCGTCTCGCGCG |
| | | TTTCGGTGATGACGGTGAAAACCTCTGACACATGCAGCTCCCGGAGACGGTCACAGCTTGTCTGT |
| | | AAGCGGATGCCGGGAGCAGACAAGCCCGTCAGGGCGCGTCAGCGGGTGTTGGCGGGTGTCGGGGC |
| | | TGGCTTAACTATGCGGCATCAGAGCAGATTGTACTGAGAGTGCACCATATGCGGTGTGAAATACC |
| | | GCACAGATGCGTAAGGAGAAAATACCGCATCAGATTGGCTAT |
| 31 | bi-shRNA Single | TTTCTTCCCCATTAGGGATTATGCTGAATTTGTATGGTTTATAGTTGTTAGAGTTTGAGGTGTTA |
| | Knockdown: (M743T | ATTCTAATTATCTATTTCAAATTTAGCAGGAAAAAAGAGAACATCACCTTGTAAAACTGAAGATT |
| | in mutant hGNE2) | GTGACCAGTCAGAATAATGTCCTGCTGGGTGCTGCCAGCACGGAGATATGTGCATCTCCGTGCTG |
| | | GCAGCACCCAGCAGGCATTATGGTGACAGCTGCCTCGGGAAGCCAAGTTGGGCTTTAAAGTGCAG |
| | | GGCCTGCTGATGTTGAGTGCTTTTTGTTCCATACTGTGTATAGCCATCATCGAGTGAAGTAGATT |
| | | AGCATCTCCGTGCTGGCAGCACCCAGCAGGCATAAGAAGTTATGTATTCATCCAATAATTCAAGC |
| | | CAAGCAAGTATATAGGTGTTTTAATAGTTTTTGTTTTGGGGATTGTGACCAGAAGATTTTGAAAA |
| | | TTAAATATTACTGAAGATTTCGACTTCCACTGTTAAATGTACAAGATACATGAAATATTAAAGAA |
| | | AATGTGTAACTTTTTGTGTAAATACATCTTGT |
| 32 | bi-shRNA Single | TTTCTTCCCCATTAGGGATTATGCTGAATTTGTATGGTTTATAGTTGTTAGAGTTTGAGGTGTTA |
| | Knockdown: (M743T | ATTCTAATTATCTATTTCAAATTTAGCAGGAAAAAAGAGAACATCACCTTGTAAAACTGAAGATT |
| | in mutant hGNE2) | GTGACCAGTCAGAATAATGTCTGCTGGGTGCTGCCAGCACGGTAGATATGTGCATCTACCGTGCT |
| | | GGCAGCACCCAGCAGCATTATGGTGACAGCTGCCTCGGGAAGCCAAGTTGGGCTTTAAAGTGCAG |
| | | GGCCTGCTGATGTTGAGTGCTTTTTGTTCATACTGTGTGCGACCATCATCGTAGTGAAGTAGATT |
| | | AGCATCTACCGTGCTGGCAGCACCCAGCAGCATAAGAAGTTATGTATTCATCCAATAATTCAAGC |
| | | CAAGCAAGTATATAGGTGTTTTAATAGTTTTTGTTTTGGGGATTGTGACCAGAAGATTTTGAAAA |
| | | TTAAATATTACTGAAGATTTCGACTTCCACTGTTAAATGTACAAGATACATGAAATATTAAAGAA |
| | | AATGTGTAACTTTTTGTGTAAATACATCTTGT |

In some embodiments, an expression vector comprises a first nucleic acid encoding a wild-type GNE (e.g. hGNE1 or hGNE2) and a second nucleic acid encoding at least one bifunctional short hairpin RNA (bi-shRNA) capable of hybridizing to a region of an mRNA transcript encoding a mutant GNE is referred to as a wtGNE/bishRNA^{mutGNE} expression vector.

In some embodiments, the first nucleic acid and the second nucleic acid are operably linked to a promoter. In some embodiments, the promoter is a cytomegalovirus (CMV) promoter. In some embodiments, the CMV promoter is a mammalian CMV promoter. In some embodiments, the mammalian CMV promoter comprises a CMV immediate early (IE) 5′ UTR enhancer sequence and a CMV IE Intron A.

In some embodiments, a nucleotide sequence encoding a picornaviral 2A ribosomal skip peptide sequence is intercalated between the first and the second nucleic acid inserts.

### EXAMPLES

### Example 1

A group of at least 10 people diagnosed with hereditary inclusion body myopathy are treated with a pharmaceutical composition containing a therapeutically effective amount of the bifunctional shRNA sequence of SEQ ID NO:3 that knocks down the expression of the mutant GNE in the patients. Each patient is administered the pharmaceutical composition via intramuscular injection. Either substantially simultaneously (i.e., within a few minutes (e.g., within 20 minutes) of administering the bifunctional shRNA), or after administering the bifunctional shRNA (i.e., at least 20 minutes after), each patient is also administered a pharmaceutical composition containing a therapeutically effective amount of a wild-type GNE-encoding nucleic acid sequence (e.g., SEQ ID NO:1) or a vector containing the wild-type GNE-encoding nucleic acid sequence (e.g., SEQ ID NO:11). The frequency and amount of each administration can be determined by a physician. To assess the effectiveness of the treatment, each patient's muscle function can be evaluated.

### Example 2: Results

Design of Dual Function Plasmids for Both the Expression of Wild-Type hGNE2 and Knockdown of the Expression of M743T Mutation

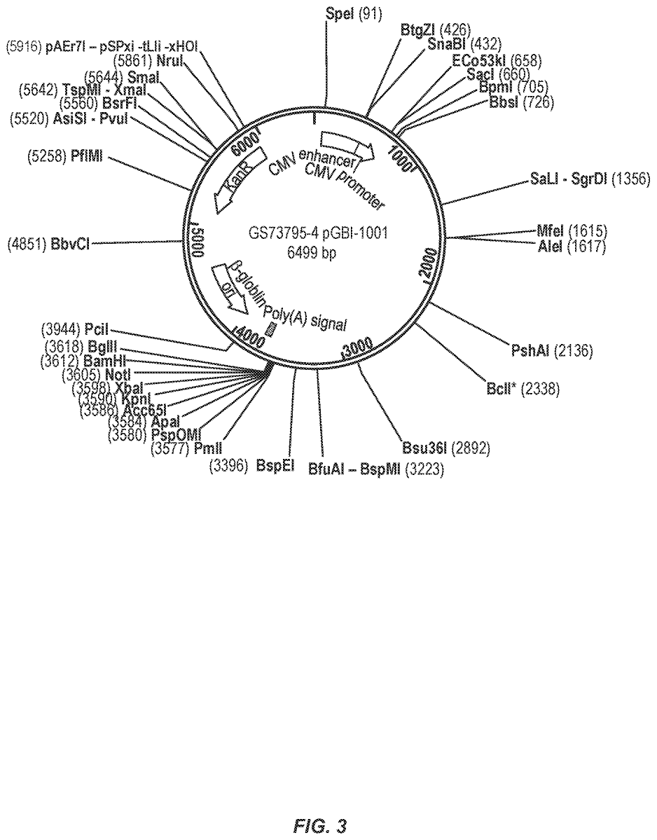

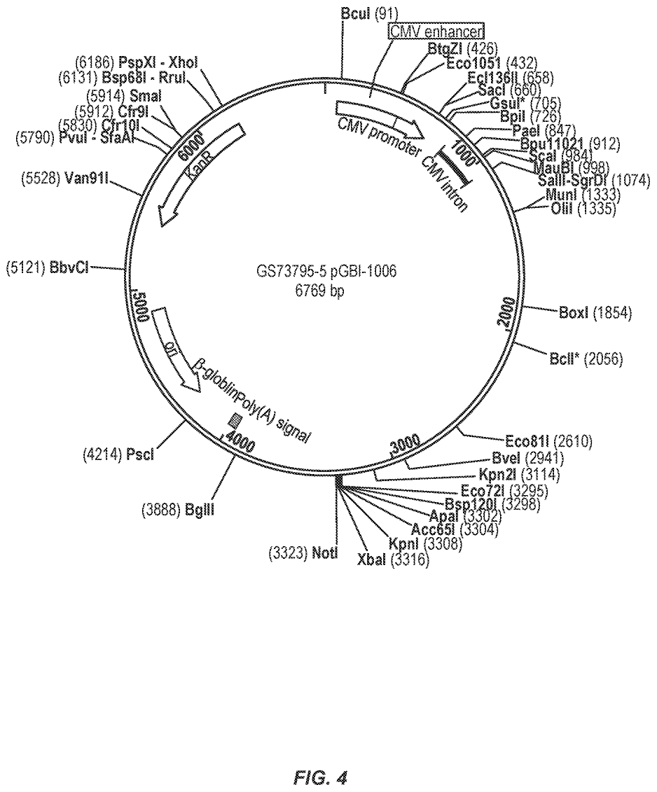

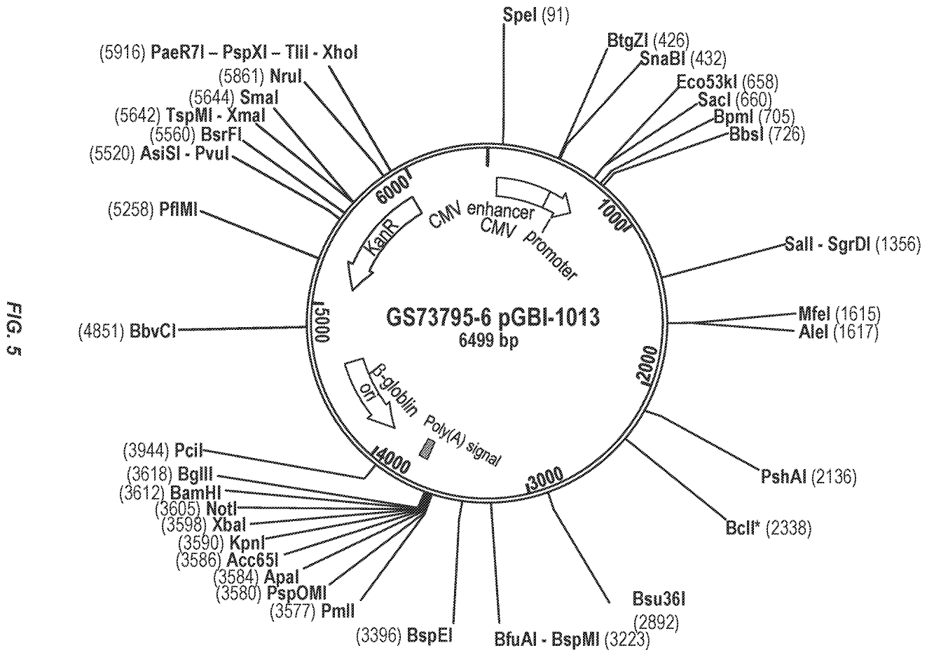

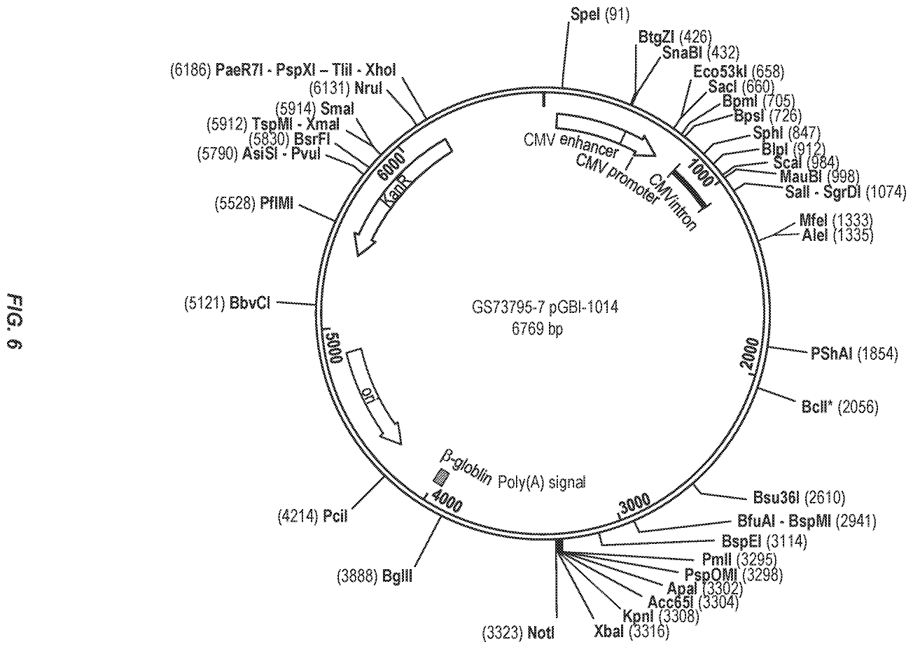

CMV promoter-based expression vector, pUMVC3, was designed to express wild-type hGNE2 and bi-shRNA targeting M743T mutation with a single plasmid. Two bi-shRNAs were designed to test specificity of M743T mutation knockdown. Four dual function plasmids were designed with two bi-shRNA sequences either in front of hGNE2 mRNA unit or behind hGNE2 mRNA unit, they were assigned with sequence code number pGBI-1001, pGBI-1006, pGBI-1013 and pGBI-1014. The maps are shown in FIGS. 3-6. The sequences are shown as SEQ ID NOS:20-23 in Table 1.

Strep Tagged Expression Plasmids to Monitor M743T Mutant mRNA Specific Knockdown

To enabling the assessment of M743T specific knockdown at the protein level, eight residue Strep Tag II sequence was inserted into the amino terminus of either the wild-type hGNE2 expression plasmids or the M743T mutant expression plasmids, they are pGBI-1011 and pGBI-1012, respectively.

Restriction Fragment Length Polymorphism (RFLP) Assay to Differentiate M743T Mutant mRNA Transcript from the Wild-Type mRNA Transcript

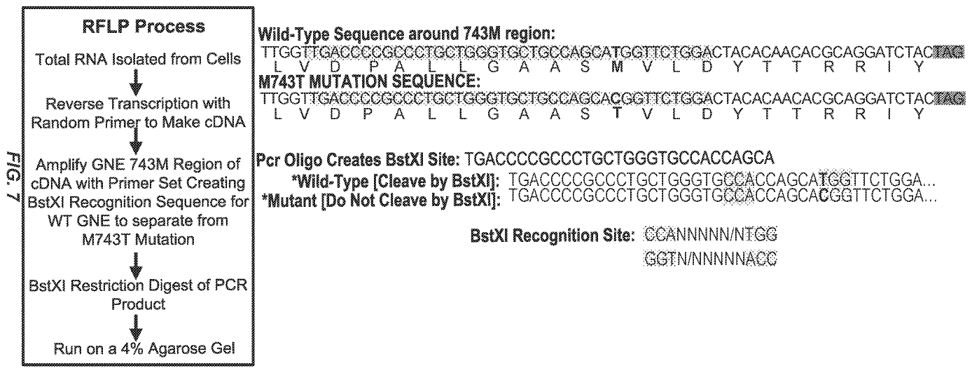

The dual function plasmids were designed to express wild-type hGNE2 while knocking down the expression of M743T mutant. To assess M743T mutant mRNA knockdown without affecting the wild-type mRNA, a method was required to differentiate the M743T mutant mRNA from the wild-type mRNA. We developed a RT-PCR based RFLP strategy to do so. The RFLP strategy is illustrated in FIG. 7. The 3′ end of restriction enzyme BstXI recognition sequence is TGG which recognizes wild-type sequence GGATGG (SEQ ID NO:33), not M743T mutation sequence GGACGG (SEQ ID NO:34). The M743T mutant mRNA RT-PCR product was not recognized by BstXI, thus resistant to digestion, while wild-type mRNA RT-PCR product was recognized by BstXI, thus was digested by BstXI. A forward hGNE2 specific PCR primer with modification to include the 5′ end sequence of BstXI recognition site was designed to enabling BstXI digest. The RFLP is schematically illustrated in the FIG. 7.

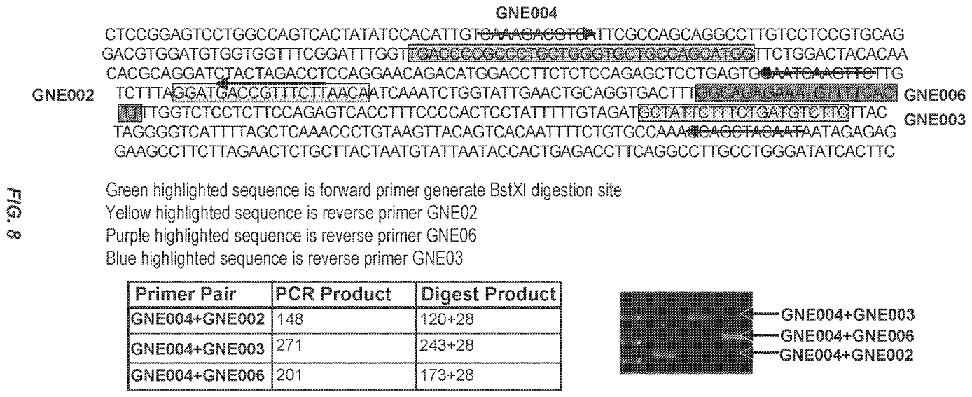

Three reverse primers were designed to generate different sizes of RT-PCR products. The primer location and their respective PCR products before and after BstXI digest are illustrated in FIG. 8. The RT-PCR was tested, and the expected RT-PCR product was shown on the lower right panel of FIG. 8.

Dual Function Plasmids Showed Knock Down M743T Mutant without Affecting Wild-Type Expression

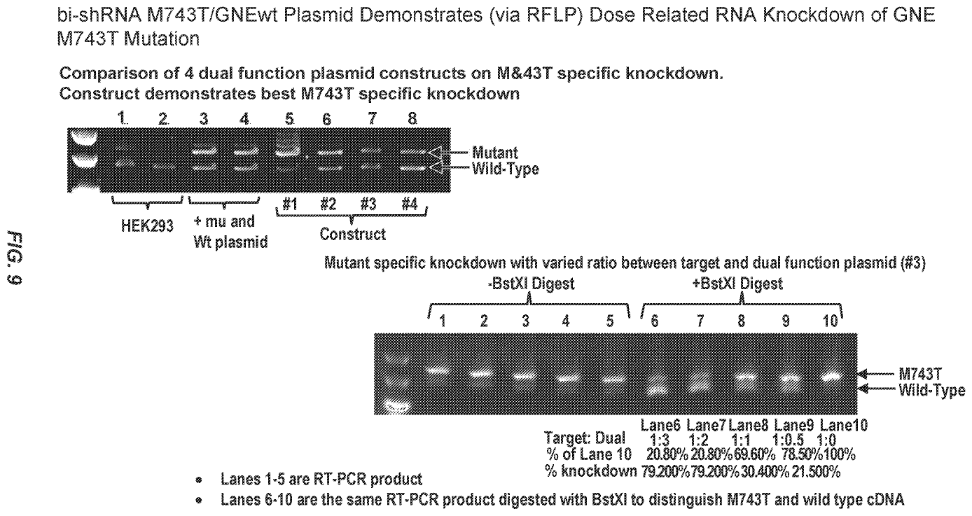

To test expression and knockdown effect of dual-function plasmids, we co-transfected hGNE2 M743T mutation expression plasmid either with hGNE2 wild-type expression plasmid or with four designed dual-function expression plasmids. The RFLP data is shown in FIG. 9. The expression of endogenous hGNE2 mRNA of HEK293 cells was relatively low and with wild type only (upper panel, lanes 1 and 2, of FIG. 9). Co-transfection of wild-type hGNE2 and M743T mutant expression plasmids in HEK293 cells resulted in expression of both M743T mutant mRNA (upper panel, lanes 3 and 4, upper band, of FIG. 9) and hGNE2 wild-type mRNA (upper panel, lanes 3 and 4, lower band, of FIG. 9). Co-transfection of dual-function plasmids (expresses wild-type hGNE2 and knockdown M743T) with M743T mutant expression plasmid showed varied ratio of wild-type and M743T mRNA. Dual function plasmid #1 (SEQ IDNO:20) co-transfected cells had more M743T transcripts than wild-type transcripts (upper panel, lane 5, of FIG. 9), while dual-function plasmid #4 (SEQ IDNO:23) co-transfected cells had less M743T transcripts than wild-type transcripts (upper panel, lane 8, of FIG. 9).

To further analyze the dose effect, we modulated the ratio of M743T mutant expression plasmid to the dual-function plasmid #3 (SEQ ID NO:22) for co-transfection. Without dual-function plasmid, the M743T mutant expression plasmid transfected cells showed predominantly M743T mutant (lower panel, lane 10, of FIG. 9). Co-transfection with increasing dual-function plasmid #3 (SEQ ID NO:22) resulted in increased wild-type mRNA and decreased M743T mutant mRNA expression (lower panel, lanes 9, 8, 7 and 6, of FIG. 9). Without BstXI digest, the total mRNA transcripts for all 5 samples were about even (lower panel, lane 1-5, of FIG. 9). With a 1:2 or 1:3 ratio of M743T mutant expression plasmid to dual-function plasmid, the M743 mutant transcripts knockdown was about 80%. Interestingly, the total transcript for all conditions appeared to be the same (lower panel, lanes 1-5, of FIG. 9) and yet the ratio of wild type to M743T mutant mRNA varied according to the ratio of input plasmids (lower panel, lanes 6-10, of FIG. 9). There could be expression regulations at mRNA level.

Dual Function Plasmids Reduced hGNE2 M743T Mutation Protein without Affecting Wild Type Expression.

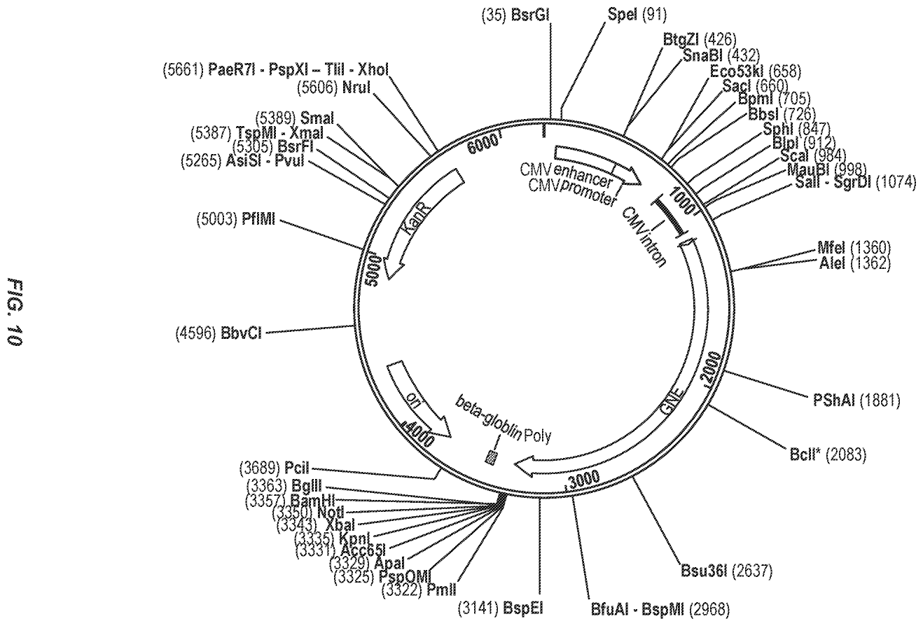

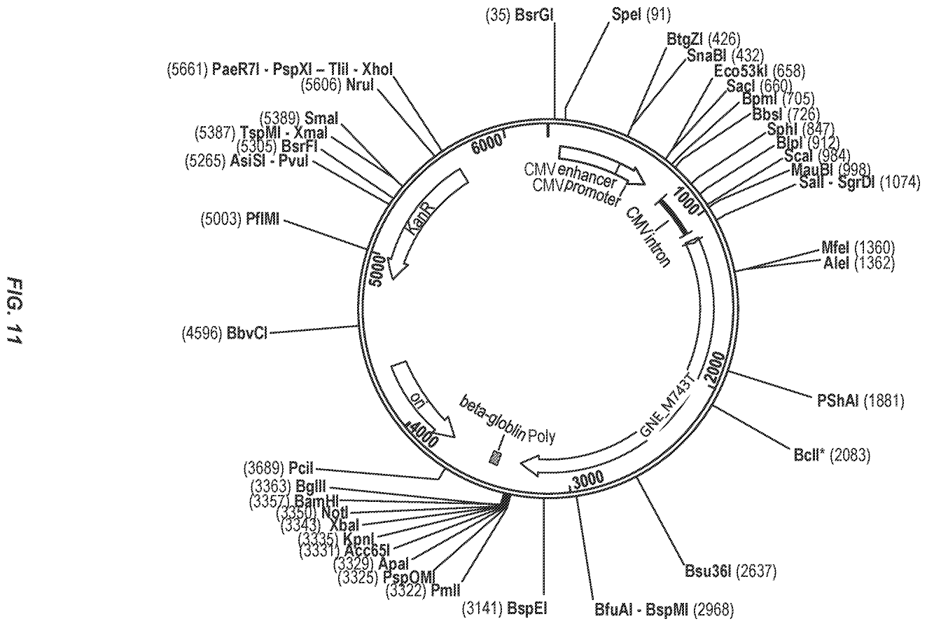

Expression plasmids express Strep tagged hGNE2 protein (pGBI-1011, FIG. 10, SEQ ID NO:24), or Strep tagged M743T mutation protein (pGBI-1012, FIG. 11, SEQ ID NO:25) were constructed. The 8 residue Strep tags were engineered to be at the amino terminus of each protein. Co-transfection of Strep tagged expression plasmids allowed us to selectively analyze M743T mutant specific protein or hGNE2 wild-type specific protein in the host or hGNE2 dual-function background.

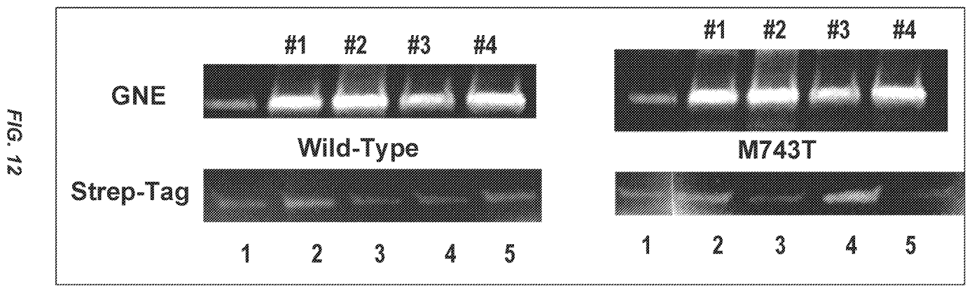

Four dual-function plasmids were co-transfected with either strep tagged wild-type hGNE2 expression plasmid or strep tagged M743T mutant hGNE2 expression plasmid in HEK293 cells. The dual-function plasmid to tagged expression plasmid were transfected at 1:2 ratio. Total protein from transfected cells were harvested 48 hours post transfection and equal amounts of total protein were ran on gel for Western analysis. The results of Western analysis are shown on FIG. 12. The left set of panels of FIG. 12 were from cells co-transfected with Strep tagged wild type hGNE2 expression plasmid and the right set of panels were from cells co-transfected with Strep tagged M743T mutant expression plasmid. The upper panels were detected with GNE antibody, and the lower panels were detected with anti-Strep antibody.

Lane 1 of each set of samples was from cells co-transfection with pUMVC3 empty vector control (no hGNE2 or M743T mutant expression), which shows that the total hGNE2 expression was low. Dual-function plasmids expressed an abundance of hGNE2 proteins (upper left panel, lanes 2-5) without significantly affecting tagged hGNE2 wild-type protein (lower left panel, lanes 2-5). On the other hand, the dual-function plasmids #2 and #4 (SEQ ID NOS: 21 and 23, respectively) significantly reduced the tagged M743T hGNE2 protein (lower right panel).

In conclusion, with in vitro analysis, all four dual-function plasmids expressed wild-type hGNE2 well both at mRNA and protein level. Four different constructs had slightly different efficiency in M743T mutant expression knockdown without affecting the wild-type hGNE2 expression. M743T mutant specific knockdown was assessed at about 80% efficiency in vitro.

### Example 3: Discussion

Gene replacement therapy with GNEwt DNA is hypothesized to enhance hGNE2 activity and reduce myopathy development in patients with GNE myopathy. We have previously demonstrated in preclinical testing and with management of one severe GNE myopathy patient via single patient IND that plasmid delivery of GNEwt is feasible and well tolerated. Furthermore, there was demonstrated evidence of benefit related to GNEwt muscle delivery. However, with feedback regulation, GNEwt gene replacement alone may or may not fully restore GNE function. We thus propose a “push and pull” strategy to express GNEwt in combination with mutant hGNE2 knockdown using bi-shRNA. The combined expression of GNEwt and hGNE2 mutant specific bi-shRNA knockdown was thus constructed as a single expression vector.

The bi-shRNA based technology is an exquisitely specific knockdown technology, allowing for the initial designed construct to target the M743T hGNE2 mutation. Bennmann et al. demonstrated the change from methionine to threonine in the M743T mutation created a site for O-GlcNAcylation enabling increased O-GlcNAcylation of GNEmu in comparison to GNEwt protein. Moreover, the aberrant post translational modification of GNE does disrupt GNE enzymatic activity. Furthermore, the aberrant O-GlcNAcylated GNE has a longer half-life than the GNEwt counterpart [14]. These factors provide additional competitive advantage of hGNE2 M743T variant protein compared to GNEwt protein and support the “push and pull” strategy to lower hGNE2 M743T activity while concurrently enhancing GNEwt protein expression.

To test the specificity and effectiveness of this strategy in vitro, we constructed a GNEwt/bi-shRNA-M743T plasmid. Replacement of GNEwt activity should repair sialic acid synthesis disruption [9]. Results with the DMRV-hIMB murine model demonstrate that muscle atrophy is improved with prophylactic sialic acid metabolic replacement [19]. Supplementation with ManNAc beginning at 5-6 weeks through the typical age when symptoms would be found (54-57 weeks) demonstrated increased survival, as well as motor performance, contractile muscle and muscle pathology improvement. Clinically, treatment with sialic acid precursors like Neu5Ac or extended release aceneuramic acid (Ace-ER) improved sialic acid concentration, however it has not demonstrated meaningful clinical benefit. In a Phase 3 double-blind placebo-controlled trial evaluating the efficacy and safety of Ace-ER, no clinical benefit defined as improvement of muscle strength was demonstrated compared to placebo [20].

Several FDA approved products involve plasmid delivery have been proven to justify FDA recommendation for use in clinical management [17, 21]. Plasmid construction can be engineered to provide for prolonged gene expression without integration in the genome or risk of replication [22, 23]. Persistence elements (i.e., inverted terminal repeats [24, 25]) can be incorporated into plasmid design to transiently prolong gene expression in vitro and in vivo thereby minimizing time of treatment timepoints and frequency of patient treatments. Inducible promoters can also be considered to promote expression in the presence of a positive regulator or in the absence of a negative regulator [26]. Taking sequence elements from a privileged gene to include in our expression plasmid, for example, can lead to as much as a 70-fold increased gene expression in vivo [27]. Such an approach may be utilized in a future plasmid design if short-term hGNE2 expression and hGNE2 M743T knockdown demonstrates evidence of muscle function enhancement.

GNE protein is expressed in skeletal muscle at similar levels in GNE myopathy patients as well as normal subjects. As previously described, impaired GNE function, not lack of expression, appears to be the key pathogenic factor involving GNE myopathy [49]. Several different GNE mutations in fact have demonstrated altered activity of GNE enzyme function as assessed by downstream enzyme kinetics [12]. All mutations however appear to retain a minimal amount of activity relative to the GNEwt enzyme which may relate to gradual muscle function deterioration over many years rather than sudden myopathy. In support of this, Savelkoul et al. [11] suggested that GNE myopathy defects related to sialylation appear gradually in muscle tissue in relation to disruptions in GNE enzyme function.

The mechanism of muscle weakness symptomology however in relation to GNE mutation is not entirely known. Hypoglycosylation of a-dystroglycan, a central protein of the skeletal muscle dystrophin-glycoprotein complex, may in part be related to myopathy development. a-Dystroglycan plays a significant role in anchoring the extracellular matrix to the cytoskeleton of the sarcolemma. Disruption of a-dystroglycan binding to the muscle extracellular matrix and the cytoskeleton may lead to destabilization of the sarcolemma during contraction. In time this may lead to deterioration in muscle strength and function. Work by Huizing involving four GNE myopathy patient assessments appears supportive of this mechanism [5].

M743T is only one of more than 150 genetic variations involving GNE myopathy syndrome [28]. RNAi targeted therapy only impacts the specific mutation, in this case GNE M743T, not all variants. However, clinical proof of principle in M743T hGNE2 myopathy patients will likely open the door to other plasmid constructs to impact other GNE myopathy variants.

Studies have reported that despite decreased biochemical activity of variant GNE, the overall sialic acid levels in patients can be unaffected [29] or unrelated to clinical significance. Mouse models carrying the GNE p.M743T alleles generated through homologous recombination may not develop myopathy [30, 31]. Although the transgenic mouse model Gne(−/−) hGNE D176V-Tg, which expressed the human GNE p.D207V (hGNE2) variant is representative of GNE myopathy [32].

It is possible that other genetic signal patterns involved in GNE myopathy also relate to the pathogenesis time of onset and severity. Prevalence of GNE myopathy is suggested by Celeste et al. and is predicted to involve ~40,000 patients worldwide. However, only about 800 patients with clinical symptomatology have been reported, the reason for the discrepancy is unclear [28]. It could be due to under-diagnosis particularly outside USA, Japan and Europe and may be related to null mutation in GNE likely being associated with embryonic lethality. In the absence of a full genetic analysis of all patients and family, the possibility that some patients may not develop several symptomatology also cannot be excluded.

Regardless, symptomatic patients with M743T GNE myopathy being managed show decreased, but not absent, enzyme activity. Data analysis reveals that a 30% reduced UDPGlcNAc 2-epimerase activity has been reported for various GNE myopathy patients [29, 33] including up to a 45% reduced ManNAc kinase activity [34]. Reduced activity of patients with the M743T variant has also suggested relationship in the folding of the GNE variant protein [3, 13]. Weidemann et al. [13] demonstrated a reduced solubility and lower isoelectric point of the M743T variant protein in comparison to the GNEwt protein and further demonstrated that the phosphorylation of the M743T variant protein influenced its isoelectric point. These observations likely relate to the results described by Bennmann earlier which demonstrate competitive advantage for GNE M743T variant protein over GNEwt protein highlighting the modulation of O-GlcNAcylation seen with GNE M743T variant protein. O-GlcNAcylation plays a significant role in protein stability, enzyme activities and protein structure conformation. These alterations can also influence enzyme specificity [35-38]. Moreover, it has also been shown that the half-life time of M743T variant related to 0-GlcNAcylation provides superior approach to M743T activity over GNEwt protein activity thereby further contributing to the pathogenesis and severity of GNE myopathy. These factors may also relate to age of disease onset and rate of debilitative onset. Evidence of relationship of disease pathogenesis relate to 0-GlcNAc modulation has also been observed in oncology involving cancer activity, diabetes and neurodegenerative disease [39][40][41].

### REFERENCES

1. Huizing, M. and D. M. Krasnewich, Hereditary inclusion body myopathy: a decade of progress. Biochim Biophys Acta, 2009. 1792(9): p. 881-7.
2. Kaback, M., et al., Genetic screening in the Persian Jewish community: A pilot study. Genet Med, 2010. 12(10): p. 628-33.
3. Stasche, R., et al., A bifunctional enzyme catalyzes the first two steps in N-acetylneuraminic acid biosynthesis of rat liver. Molecular cloning and functional expression of UDP-N-acetyl-glucosamine 2-epimerase N-acetylmannosamine kinase. J Biol Chem, 1997. 272(39): p. 24319-24.
4. Varki, A., Sialic acids as ligands in recognition phenomena. FASEB J, 1997. 11(4): p. 248-55.
5. Huizing, M., et al., Hypoglycosylation of alpha-dystroglycan inpatients with hereditary IBMdue to GNE mutations. Mol Genet Metab, 2004. 81(3): p. 196-202.
6. Keppler, O. T., et al., UDP-GlcNAc 2-epimerase: a regulator of cell surface sialylation. Science, 1999. 284(5418): p. 1372-6.
7. Ricci, E., et al., NCAM is hyposialylated in hereditary inclusion body myopathy due to GNE mutations. Neurology, 2006. 66(5): p. 755-8.
8. Langer, G. A., The structure and function of the myocardial cell surface. Am J Physiol, 1978. 235(5): p. H461-8.
9. Jay, C. M., et al., Hereditary Inclusion Body Myopathy (HIBM2). Gene Regul Syst Bio, 2009. 3: p. 181-90.
10. Krause, S., et al., GNE protein expression and subcellular distribution are unaltered in HIBM. Neurology, 2007. 69(7): p. 655-9.
11. Savelkoul, P. J., et al., Normal sialylation of serum N-linked and O-GalNAc-linked glycans in hereditary inclusion-body myopathy. Mol Genet Metab, 2006. 88(4): p. 389-90.
12. Penner, J., et al., Influence of UDP-GlcNAc 2-epimerase ManNAc kinase mutant proteins on hereditary inclusion body myopathy. Biochemistry, 2006. 45(9): p. 2968-77.
13. Weidemann, W., et al., Biochemical characterization of the M712T-mutation of the UDP-N-acetylglucosamine 2-epimerase N-acetyl-mannosaminekinase in hereditary inclusion body myopathy. Neuromuscul Disord, 2011. 21(12): p. 824-31.
14. Bennmann, D., et al., Aberrant O-GlcNAcylation disrupts GNE enzyme activity in GNE myopathy. FEBS J, 2016. 283(12): p. 2285-94.
15. Jay, C., et al., Preclinical assessment ofwt GNE gene plasmid for management of hereditary inclusion body myopathy 2 (HIBM2). Gene Regul Syst Bio, 2008. 2: p. 243-52.
16. Phadke, A. P., et al., Safety and in vivo expression of a GNE-transgene: a novel treatment approachfor hereditary inclusion body myopathy-2. Gene Regul Syst Bio, 2009.3: p. 89-101.
17. Nemunaitis, G., et al., Hereditary inclusion body myopathy: single patient response to GNE gene Lipoplex therapy. J Gene Med, 2010. 12(5): p. 403-12.
18. Nemunaitis, G., et al., Hereditary inclusion body myopathy: single patient response to intravenous dosing of GNE gene lipoplex. Hum Gene Ther, 2011. 22(11): p. 1331-41.
19. Malicdan, M. C., et al., Prophylactic treatment with sialic acid metabolites precludes the development of the myopathic phenotype in the DMRV-hIBM mouse model. Nat Med, 2009. 15(6): p. 690-5.
20. Lochmüller, H., et al., A phase 3 randomized study evaluating sialic acid extended-releasefor GNE myopathy. Neurology, 2019. 92(18): p. e2109-e2117.
21. Curreri, A., et al., RNA therapeutics in the clinic. Bioengineering & Translational Medicine, 2023. 8(1): p. e10374.
22. Ledwith, B. J., et al., Plasmid DNA vaccines: investigation of integration into host cellular DNA following intramuscular injection in mice. Intervirology, 2000. 43(4-6): p. 258-72.
23. Chen, Z., et al., Minimized antibiotic-free plasmid vector for gene therapy utilizing a new toxin-antitoxin system. Metabolic Engineering, 2023. 79: p. 86-96.
24. Aksentij evich, I., et al., In vitro and in vivo liposome-mediated gene transfer leads to human MDR1 expression in mouse bone marrow progenitor cells. Hum Gene Ther, 1996. 7(9): p. 1111-22.
25. Baudard, M., et al., Expression of the human multidrug resistance and glucocerebrosidase cDNAs from adeno-associated vectors: efficient promoter activity ofAAV sequences and in vivo delivery via liposomes. Hum Gene Ther, 1996. 7(11): p. 1309-22.
26. Vilaboa, N. and R. Voellmy, Deliberate regulation of therapeutic transgenes, in Gene and Cell Therapy: Therapeutic Mechanisms and Strategies, 3rd edn, N. S. Templeton, Editor. 2008, Taylor & Francis/CRC Press, Inc.: Boca Raton, FL, 2008. p. 619-636.
27. Lu, H., et al., Enhanced gene expression in breast cancer cells in vitro and tumors in vivo. Mol Ther, 2002. 6(6): p. 783-92.
28. Celeste, F. V., et al., Mutation update for GNE gene variants associated with GNE myopathy. Hum Mutat, 2014. 35(8): p. 915-26.
29. Salama, I., et al., No overall hyposialylation in hereditary inclusion body myopathy myoblasts carrying the homozygous M712T GNE mutation. Biochem Biophys Res Commun, 2005. 328(1): p. 221-6.
30. Galeano, B., et al., Mutation in the key enzyme of sialic acid biosynthesis causes severe glomerular proteinuria and is rescued by N-acetylmannosamine. J Clin Invest, 2007. 117(6): p. 1585-94.
31. Ito, M., et al., Glycoprotein hyposialylation gives rise to a nephrotic-like syndrome that is prevented by sialic acid administration in GNE V572L point-mutant mice. PLoS One, 2012. 7(1): p. e29873.
32. Malicdan, M. C., et al., A Gne knockout mouse expressing human GNE DI76V mutation develops features similar to distal myopathy with rimmed vacuoles or hereditary inclusion body myopathy. Hum Mol Genet, 2007. 16(22): p. 2669-82.
33. Hinderlich, S., et al., The homozygous M712T mutation of UDP-N-acetylglucosamine 2-epimerase N-acetylmannosamine kinase results in reduced enzyme activities but not in altered overall cellular sialylation in hereditary inclusion body myopathy. FEBS Lett, 2004. 566(1-3): p. 105-9.
34. Sparks, S. E., et al., Use of a cell-free system to determine UDP-N-acetylglucosamine 2-epimerase and N-acetylmannosamine kinase activities in human hereditary inclusion body myopathy. Glycobiology, 2005. 15(11): p. 1102-10.
35. Miranda, F. F., et al., Structural and stability effects ofphosphorylation: Localized structural changes in phenylalanine hydroxylase. Protein Sci, 2004. 13(5): p. 1219-26.
36. Chu, C S., et al., O-GlcNAcylation regulates EZH2 protein stability and function. Proc Natl Acad Sci USA, 2014. 111(4): p. 1355-60.
37. Olivier-Van Stichelen, S., et al., O-GlcNAcylation stabilizes beta-catenin through direct competition with phosphorylation at threonine 41. FASEB J, 2014. 28(8): p. 3325-38.
38. Charoensuksai, P., et al., O-GlcNAcylation of co-activator-associated arginine methyltransferase 1 regulates its protein substrate specificity. Biochem J, 2015. 466(3): p. 587-99.
39. Buse, M. G., et al., Enhanced O-GlcNAc protein modification is associated with insulin resistance in GLUT1-overexpressing muscles. Am J Physiol Endocrinol Metab, 2002. 283(2): p. E241-50.
40. Slawson, C. and G. W. Hart, O-GlcNAc signalling: implicationsfor cancer cell biology. Nat Rev Cancer, 2011. 11(9): p. 678-84.
41. Lazarus, B. D., D. C. Love, and J. A. Hanover, O-GlcNAc cycling: implicationsfor neurodegenerative disorders. Int J Biochem Cell Biol, 2009. 41(11): p. 2134-46.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. An expression vector comprising a bifunctional short hairpin RNA (shRNA) sequence specific for knockdown of a mutant GNE, wherein the bifunctional shRNA sequence encodes a nucleic acid sequence capable of hybridizing to one or more regions of an mRNA transcript encoding the mutant GNE to inhibit the expression of the mutant GNE via RNA interference,
wherein the bifunctional shRNA comprises a first stem-loop structure that comprises an siRNA component and a second stem-loop structure that comprises a miRNA component.

2. The expression vector of claim 1, wherein the one or more regions of the mRNA transcript encoding the mutant GNE are selected from nucleotides 526-528, 1714-1716, and 2134-2136 of SEQ ID NO:16.

3. The expression vector of claim 1, wherein the siRNA component functions in a cleavage-dependent manner and the miRNA component functions in a cleavage-independent manner.

4. The expression vector of claim 1, wherein the bifunctional shRNA sequence is operably linked to a promoter.

5. (canceled)

6. The expression vector of claim 1, wherein the bifunctional shRNA sequence comprises a sequence having at least 90% identity to any one of SEQ ID NOS:3-6, 31, and 32.

7. (canceled)

8. A composition comprising the expression vector of claim 1 and a wild-type GNE-encoding nucleic acid sequence comprising SEQ ID NO:1 or 27.

9. (canceled)

10. The composition of claim 8, wherein the wild-type GNE-encoding nucleic acid sequence and the bifunctional shRNA sequence are provided in, disposed within, or connected to one or more liposomes or lipid nanoparticles, and wherein the liposome or the lipid nanoparticle comprises one or more agents capable of recognizing and binding to a muscle cell or a component thereof.

11. The composition of claim 8, wherein the wild-type GNE-encoding nucleic acid sequence and the bifunctional shRNA sequence comprises a promoter operably connected to the wild-type GNE-encoding nucleic acid sequence and the bifunctional shRNA sequence.

12-14. (canceled)

15. A method for ameliorating the effects of hereditary inclusion body myopathy, which comprises the steps of:
(i) identifying a human subject with hereditary inclusion body myopathy; and
(ii) providing the human subject with effective amounts of a wild-type GNE-encoding nucleic acid sequence and a bifunctional shRNA sequence that knocks down the expression of a mutant GNE in the human subject by administration at a location with hereditary inclusion body myopathy,
wherein the providing step is by administration at a location with hereditary inclusion body myopathy, and the wild-type GNE-encoding nucleic acid sequence comprises SEQ ID NO:1 and the bifunctional shRNA sequence comprises a sequence having at least 90% identity to any one of SEQ ID NOS:3-6; or
wherein the providing step is by administration at a location with hereditary inclusion body myopathy or systemically, and the wild-type GNE-encoding nucleic acid sequence comprises SEQ ID NO:27 and the bifunctional shRNA sequence comprises a sequence having at least 90% identity to any one of SEQ ID NOS:31 and 32.

16. (canceled)

17. The method of claim 15, wherein the wild-type GNE-encoding nucleic acid sequence and/or the bifunctional shRNA sequence is provided in, disposed within, or connected to one or more liposomes or lipid nanoparticles, and wherein the liposome or the lipid nanoparticle comprises one or more agents capable of recognizing and binding to a muscle cell or a component thereof.

18. The method of claim 15, wherein the administration is via intramuscular administration to human muscle cells.

19. The method of claim 15, wherein the wild-type GNE-encoding nucleic acid sequence comprises a promoter operably connected to the wild-type GNE-encoding nucleic acid sequence.

20-22. (canceled)

23. A method for modulating the production of sialic acid in a human, which comprises the steps of:
providing a human subject in need of treatment of a hereditary inclusion body myopathy;
providing a human wild-type GNE-encoding nucleic acid sequence and a bifunctional shRNA sequence that knocks down the expression of a mutant GNE in the human subject by administration at a location with hereditary inclusion body myopathy,
wherein (i) the wild-type GNE-encoding nucleic acid sequence comprises SEQ ID NO:1 and the bifunctional shRNA sequence comprises a sequence having at least 90% identity to any one of SEQ ID NOS:3-6: or (ii) the wild-type GNE-encoding nucleic acid sequence comprises SEQ ID NO:27 and the bifunctional shRNA sequence comprises a sequence having at least 90% identity to any one of SEQ ID NOS:31 and 32.

24. (canceled)

25. The method of claim 23, wherein the wild-type GNE-encoding nucleic acid sequence and the bifunctional shRNA sequence is provided in, disposed within, or connected to one or more liposomes or lipid nanoparticles, and wherein the liposome or the lipid nanoparticle comprises one or more agents capable of recognizing and binding to a muscle cell or a component thereof.

26. The method of claim 23, wherein the administration is via intramuscular administration to human muscle cells.

27. The method of claim 23, wherein the wild-type GNE-encoding nucleic acid sequence comprises a promoter operably connected to the wild-type GNE-encoding nucleic acid sequence.

28-30. (canceled)

31. A method for expressing a wild-type GNE and a bifunctional shRNA that knocks down the expression of a mutant GNE in a human subject with the mutant GNE, comprising:
administering a wild-type GNE-encoding sequence and a bifunctional shRNA sequence at a location in a muscle with hereditary inclusion body myopathy, and
wherein (i) the wild-type GNE-encoding nucleic acid sequence comprises SEQ ID NO:1 and the bifunctional shRNA sequence comprises a sequence having at least 90% identity to any one of SEQ ID NOS:3-6: or (ii) the wild-type GNE-encoding nucleic acid sequence comprises SEQ ID NO:27 and the bifunctional shRNA sequence comprises a sequence having at least 90% identity to any one of SEQ ID NOS:31 and 32.

32. (canceled)

33. The method of claim 31, wherein the wild-type GNE-encoding nucleic acid sequence and the bifunctional shRNA sequence are provided in, disposed within, or connected to one or more liposomes or lipid nanoparticles, and wherein the liposome or the lipid nanoparticle comprises one or more agents capable of recognizing and binding to a muscle cell or a component thereof.

34. The method of claim 31, wherein the administering is via intramuscular administration to human muscle cells.

35. The method of claim 31, wherein the wild-type GNE-encoding nucleic acid sequence comprises a promoter operably connected to the wild-type GNE-encoding nucleic acid sequence.

36-38. (canceled)


SEQUENCE LISTING (42 sequences)

SEQ ID NO: 1 - DNA, 2169 bp, Homo sapiens
  source 1..2169: mol_type = genomic DNA
atggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactag

SEQ ID NO: 2 - protein, 722 aa, Homo sapiens
MEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 3 - DNA, 1055 bp, synthetic construct
  source 1..1055: mol_type = other DNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcctgctgggtgctgccagcacggagatatgtgcatctccgtgctggcagcacccagcaggcattatggtgacagctgcctcgggaagccaagttgggctttaaagtgcagggcctgctgatgttgagtgctttttgttccatgctgtgtattgccatcatcgagtgaagtagattagcatctccgtgctggcagcacccagcaggcataagaagttatgtattcatccaataattcaagccaagcaagtatataggtgttttaatagtttttgtttgcagtcctctgttatccatgtgtgaggaccatgttcagaagaatgtactgaacatggtcctcacacatggattggtggcctgctatttccttcaaatgaatgatttttactaattttgtgtacttttattgtgtcgatgtagaatctgcctggtctatctgatgtgacagcttctgtagcactatatctgtgtacagactatgatctgtttagttatcagaacatggtcctcacacatggattactgctagctgtagaactccagcttcggcctgtcgcccaatcaaactgtcctgttactgaacactgttctatggttcagaactgggccaccttgttctggctgtgatattctgccagaacaaggtggcccagttctgctgtggtagtgaaaagtctgtagaaaagtaagggaaactcaaacccctttctacacatcactggttaacattgaattgccgtttctgtatggcagaacaaggtggcccagttctggagtttggtggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 4 - DNA, 567 bp, synthetic construct
  source 1..567: mol_type = other DNA
tttcttccccattagggattatgctgaatttgtatggtttatagttgttagagtttgaggtgttaattctaattatctatttcaaatttagcaggaaaaaagagaacatcaccttgtaaaactgaagattgtgaccagcagtcctctgttatccatgtgtgaggaccatgttcagaagaatgtactgaacatggtcctcacacatggattggtggcctgctatttccttcaaatgaatgatttttactaattttgtgtacttttattgtgtcgatgtagaatctgcctggtctatctgatgtgacagcttctgtagcactatatctgtgtacagactatgatctgtttagttatcagaacatggtcctcacacatggattactgctagctgtagaactccagcttcggcctgtcgcccaatcaaactgtcctgttactgaatggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 5 - DNA, 518 bp, synthetic construct
  source 1..518: mol_type = other DNA
tttcttccccattagggattatgctgaatttgtatggtttatagttgttagagtttgaggtgttaattctaattatctatttcaaatttagcaggaaaaaagagaacatcaccttgtaaaactgaagattgtgaccatagctgtagaactccagcttcggcctgtcgcccaatcaaactgtcctgttactgaacactgttctatggttcagaactgggccaccttgttctggctgtgatattctgccagaacaaggtggcccagttctgctgtggtagtgaaaagtctgtagaaaagtaagggaaactcaaacccctttctacacatcactggttaacattgaattgccgtttctgtatggcagaacaaggtggcccagttctggagtttggtggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 6 - DNA, 552 bp, synthetic construct
  source 1..552: mol_type = other DNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcctgctgggtgctgccagcacggagatatgtgcatctccgtgctggcagcacccagcaggcattatggtgacagctgcctcgggaagccaagttgggctttaaagtgcagggcctgctgatgttgagtgctttttgttccatgctgtgtattgccatcatcgagtgaagtagattagcatctccgtgctggcagcacccagcaggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 7 - DNA, 7525 bp, synthetic construct
  source 1..7525: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcctttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcctgctgggtgctgccagcacggagatatgtgcatctccgtgctggcagcacccagcaggcattatggtgacagctgcctcgggaagccaagttgggctttaaagtgcagggcctgctgatgttgagtgctttttgttccatgctgtgtattgccatcatcgagtgaagtagattagcatctccgtgctggcagcacccagcaggcataagaagttatgtattcatccaataattcaagccaagcaagtatataggtgttttaatagtttttgtttgcagtcctctgttatccatgtgtgaggaccatgttcagaagaatgtactgaacatggtcctcacacatggattggtggcctgctatttccttcaaatgaatgatttttactaattttgtgtacttttattgtgtcgatgtagaatctgcctggtctatctgatgtgacagcttctgtagcactatatctgtgtacagactatgatctgtttagttatcagaacatggtcctcacacatggattactgctagctgtagaactccagcttcggcctgtcgcccaatcaaactgtcctgttactgaacactgttctatggttcagaactgggccaccttgttctggctgtgatattctgccagaacaaggtggcccagttctgctgtggtagtgaaaagtctgtagaaaagtaagggaaactcaaacccctttctacacatcactggttaacattgaattgccgtttctgtatggcagaacaaggtggcccagttctggagtttggtggggattgtgaccagaagattttgaaaattaaatattactgaagatttcgacttccactgttaaatgtacaagatacatgaaatattaaagaaaatgtgtaactttttgtgtaaatacatcttgtaacggtggagggcagtgtagtctgagcagtactcgttgctgccgcgcgcgccaccagacataatagctgacagactaacagactgttcctttccatgggtcttttctgcagtcaccgtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttggttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 8 - DNA, 7037 bp, synthetic construct
  source 1..7037: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcctttcttccccattagggattatgctgaatttgtatggtttatagttgttagagtttgaggtgttaattctaattatctatttcaaatttagcaggaaaaaagagaacatcaccttgtaaaactgaagattgtgaccagcagtcctctgttatccatgtgtgaggaccatgttcagaagaatgtactgaacatggtcctcacacatggattggtggcctgctatttccttcaaatgaatgatttttactaattttgtgtacttttattgtgtcgatgtagaatctgcctggtctatctgatgtgacagcttctgtagcactatatctgtgtacagactatgatctgtttagttatcagaacatggtcctcacacatggattactgctagctgtagaactccagcttcggcctgtcgcccaatcaaactgtcctgttactgaatggggattgtgaccagaagattttgaaaattaaatattactgaagatttcgacttccactgttaaatgtacaagatacatgaaatattaaagaaaatgtgtaactttttgtgtaaatacatcttgtaacggtggagggcagtgtagtctgagcagtactcgttgctgccgcgcgcgccaccagacataatagctgacagactaacagactgttcctttccatgggtcttttctgcagtcaccgtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttggttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 9 - DNA, 6988 bp, synthetic construct
  source 1..6988: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcctttcttccccattagggattatgctgaatttgtatggtttatagttgttagagtttgaggtgttaattctaattatctatttcaaatttagcaggaaaaaagagaacatcaccttgtaaaactgaagattgtgaccatagctgtagaactccagcttcggcctgtcgcccaatcaaactgtcctgttactgaacactgttctatggttcagaactgggccaccttgttctggctgtgatattctgccagaacaaggtggcccagttctgctgtggtagtgaaaagtctgtagaaaagtaagggaaactcaaacccctttctacacatcactggttaacattgaattgccgtttctgtatggcagaacaaggtggcccagttctggagtttggtggggattgtgaccagaagattttgaaaattaaatattactgaagatttcgacttccactgttaaatgtacaagatacatgaaatattaaagaaaatgtgtaactttttgtgtaaatacatcttgtaacggtggagggcagtgtagtctgagcagtactcgttgctgccgcgcgcgccaccagacataatagctgacagactaacagactgttcctttccatgggtcttttctgcagtcaccgtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttggttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 10 - DNA, 7022 bp, synthetic construct
  source 1..7022: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcctttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcctgctgggtgctgccagcacggagatatgtgcatctccgtgctggcagcacccagcaggcattatggtgacagctgcctcgggaagccaagttgggctttaaagtgcagggcctgctgatgttgagtgctttttgttccatgctgtgtattgccatcatcgagtgaagtagattagcatctccgtgctggcagcacccagcaggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgtaacggtggagggcagtgtagtctgagcagtactcgttgctgccgcgcgcgccaccagacataatagctgacagactaacagactgttcctttccatgggtcttttctgcagtcaccgtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttggttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 11 - DNA, 6470 bp, synthetic construct
  source 1..6470: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttggttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 12 - DNA, 7525 bp, synthetic construct
  source 1..7525: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttggtttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcctgctgggtgctgccagcacggagatatgtgcatctccgtgctggcagcacccagcaggcattatggtgacagctgcctcgggaagccaagttgggctttaaagtgcagggcctgctgatgttgagtgctttttgttccatgctgtgtattgccatcatcgagtgaagtagattagcatctccgtgctggcagcacccagcaggcataagaagttatgtattcatccaataattcaagccaagcaagtatataggtgttttaatagtttttgtttgcagtcctctgttatccatgtgtgaggaccatgttcagaagaatgtactgaacatggtcctcacacatggattggtggcctgctatttccttcaaatgaatgatttttactaattttgtgtacttttattgtgtcgatgtagaatctgcctggtctatctgatgtgacagcttctgtagcactatatctgtgtacagactatgatctgtttagttatcagaacatggtcctcacacatggattactgctagctgtagaactccagcttcggcctgtcgcccaatcaaactgtcctgttactgaacactgttctatggttcagaactgggccaccttgttctggctgtgatattctgccagaacaaggtggcccagttctgctgtggtagtgaaaagtctgtagaaaagtaagggaaactcaaacccctttctacacatcactggttaacattgaattgccgtttctgtatggcagaacaaggtggcccagttctggagtttggtggggattgtgaccagaagattttgaaaattaaatattactgaagatttcgacttccactgttaaatgtacaagatacatgaaatattaaagaaaatgtgtaactttttgtgtaaatacatcttgtttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 13 - DNA, 7037 bp, synthetic construct
  source 1..7037: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttggtttcttccccattagggattatgctgaatttgtatggtttatagttgttagagtttgaggtgttaattctaattatctatttcaaatttagcaggaaaaaagagaacatcaccttgtaaaactgaagattgtgaccagcagtcctctgttatccatgtgtgaggaccatgttcagaagaatgtactgaacatggtcctcacacatggattggtggcctgctatttccttcaaatgaatgatttttactaattttgtgtacttttattgtgtcgatgtagaatctgcctggtctatctgatgtgacagcttctgtagcactatatctgtgtacagactatgatctgtttagttatcagaacatggtcctcacacatggattactgctagctgtagaactccagcttcggcctgtcgcccaatcaaactgtcctgttactgaatggggattgtgaccagaagattttgaaaattaaatattactgaagatttcgacttccactgttaaatgtacaagatacatgaaatattaaagaaaatgtgtaactttttgtgtaaatacatcttgtttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 14 - DNA, 6988 bp, synthetic construct
  source 1..6988: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttggtttcttccccattagggattatgctgaatttgtatggtttatagttgttagagtttgaggtgttaattctaattatctatttcaaatttagcaggaaaaaagagaacatcaccttgtaaaactgaagattgtgaccatagctgtagaactccagcttcggcctgtcgcccaatcaaactgtcctgttactgaacactgttctatggttcagaactgggccaccttgttctggctgtgatattctgccagaacaaggtggcccagttctgctgtggtagtgaaaagtctgtagaaaagtaagggaaactcaaacccctttctacacatcactggttaacattgaattgccgtttctgtatggcagaacaaggtggcccagttctggagtttggtggggattgtgaccagaagattttgaaaattaaatattactgaagatttcgacttccactgttaaatgtacaagatacatgaaatattaaagaaaatgtgtaactttttgtgtaaatacatcttgtttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 15 - DNA, 7022 bp, synthetic construct
  source 1..7022: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtctacccgcgcgcccgagcgcggaaaccgaggggcaggctcgcgctctgcctgcttcgtggcgcttggttcgtccctcgcccgaggagcgcggtggcggcgtgggagggagcctctgacggcgtctggaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttggtttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcctgctgggtgctgccagcacggagatatgtgcatctccgtgctggcagcacccagcaggcattatggtgacagctgcctcgggaagccaagttgggctttaaagtgcagggcctgctgatgttgagtgctttttgttccatgctgtgtattgccatcatcgagtgaagtagattagcatctccgtgctggcagcacccagcaggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgtttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 16 - DNA, 2169 bp, Homo sapiens
  source 1..2169: mol_type = genomic DNA
atggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgttcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttctgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcacggttctggactacacaacacgcaggatctactag

SEQ ID NO: 17 - DNA, 2169 bp, Homo sapiens
  source 1..2169: mol_type = genomic DNA
atggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgttcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactag

SEQ ID NO: 18 - DNA, 2169 bp, Homo sapiens
  source 1..2169: mol_type = genomic DNA
atggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttctgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactag

SEQ ID NO: 19 - DNA, 2169 bp, Homo sapiens
  source 1..2169: mol_type = genomic DNA
atggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcacggttctggactacacaacacgcaggatctactag

SEQ ID NO: 20 - DNA, 6499 bp, synthetic construct
  source 1..6499: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcctgctgggtgctgccagcacggagatatgtgcatctccgtgctggcagcacccagcaggcattatggtgacagctgcctcgggaagccaagttgggctttaaagtgcagggcctgctgatgttgagtgctttttgttccatactgtgtatagccatcatcgagtgaagtagattagcatctccgtgctggcagcacccagcaggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttcaccgtcgtcgacggtatcgataagcttgatatcgaattcgcccttatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagaagggcgaattcacgtgggcccggtaccgtatactctagagcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 21 - DNA, 6769 bp, synthetic construct
  source 1..6769: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcgcccttatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagaagggcgaattcacgtgggcccggtaccgtatactctagagcggccgcggtttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcctgctgggtgctgccagcacggagatatgtgcatctccgtgctggcagcacccagcaggcattatggtgacagctgcctcgggaagccaagttgggctttaaagtgcagggcctgctgatgttgagtgctttttgttccatactgtgtatagccatcatcgagtgaagtagattagcatctccgtgctggcagcacccagcaggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgtatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 22 - DNA, 6499 bp, synthetic construct
  source 1..6499: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtctgctgggtgctgccagcacggtagatatgtgcatctaccgtgctggcagcacccagcagcattatggtgacagctgcctcgggaagccaagttgggctttaaagtgcagggcctgctgatgttgagtgctttttgttcatactgtgtgcgaccatcatcgtagtgaagtagattagcatctaccgtgctggcagcacccagcagcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgttcaccgtcgtcgacggtatcgataagcttgatatcgaattcgcccttatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagaagggcgaattcacgtgggcccggtaccgtatactctagagcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 23 - DNA, 6769 bp, synthetic construct
  source 1..6769: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcgcccttatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagaagggcgaattcacgtgggcccggtaccgtatactctagagcggccgcggtttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtctgctgggtgctgccagcacggtagatatgtgcatctaccgtgctggcagcacccagcagcattatggtgacagctgcctcgggaagccaagttgggctttaaagtgcagggcctgctgatgttgagtgctttttgttcatactgtgtgcgaccatcatcgtagtgaagtagattagcatctaccgtgctggcagcacccagcagcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgtatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 24 - DNA, 6244 bp, synthetic construct
  source 1..6244: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcgcccttatgtggagccatccacagtttgagaagatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagaagggcgaattcacgtgggcccggtaccgtatactctagagcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 25 - DNA, 6244 bp, synthetic construct
  source 1..6244: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcgcccttatgtggagccatccacagtttgagaagatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcacggttctggactacacaacacgcaggatctactagaagggcgaattcacgtgggcccggtaccgtatactctagagcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 26 - DNA, 4412 bp, synthetic construct
  source 1..4412: mol_type = other DNA
gcgtataatggactattgtgtgctgatatgtacacctgaggacacaggtacaggactcagctgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcgcccttatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagaagggcgaattcacgtgggcccggtaccgtatactctagagcggccgcggtttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtctgctgggtgctgccagcacggtagatatgtgcatctaccgtgctggcagcacccagcagcattatggtgacagctgcctcgggaagccaagttgggctttaaagtgcagggcctgctgatgttgagtgctttttgttcatactgtgtgcgaccatcatcgtagtgaagtagattagcatctaccgtgctggcagcacccagcagcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgtatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatggaggtgagattgtgttcggcatgcctcacaggcagatctatcagcacacaattgcccattatacgc

SEQ ID NO: 27 - DNA, 2262 bp, Homo sapiens
  source 1..2262: mol_type = genomic DNA
atggaaacctatggttatctgcagagggagtcatgctttcaaggacctcatgaactctattttaagaacctctcaaaacgaaacaagcaaatcatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactag

SEQ ID NO: 28 - protein, 753 aa, Homo sapiens
METYGYLQRESCFQGPHELYFKNLSKRNKQIMEKNGNNRKLRVCVATCNRADYSKLAPIMFGIKTEPEFFELDVVVLGSHLIDDYGNTYRMIEQDDFDINTRLHTIVRGEDEAAMVESVGLALVKLPDVLNRLKPDIMIVHGDRFDALALATSAALMNIRILHIEGGEVSGTIDDSIRHAITKLAHYHVCCTRSAEQHLISMCEDHDRILLAGCPSYDKLLSAKNKDYMSIIRMWLGDDVKSKDYIVALQHPVTTDIKHSIKMFELTLDALISFNKRTLVLFPNIDAGSKEMVRVMRKKGIEHHPNFRAVKHVPFDQFIQLVAHAGCMIGNSSCGVREVGAFGTPVINLGTRQIGRETGENVLHVRDADTQDKILQALHLQFGKQYPCSKIYGDGNAVPRILKFLKSIDLQEPLQKKFCFPPVKENISQDIDHILETLSALAVDLGGTNLRVAIVSMKGEIVKKYTQFNPKTYEERINLILQMCVEAAAEAVKLNCRILGVGISTGGRVNPREGIVLHSTKLIQEWNSVDLRTPLSDTLHLPVWVDNDGNCAALAERKFGQGKGLENFVTLITGTGIGGGIIHQHELIHGSSFCAAELGHLVVSLDGPDCSCGSHGCIEAYASGMALQREAKKLHDEDLLLVEGMSVPKDEAVGALHLIQAAKLGNAKAQSILRTAGTALGLGVVNILHTMNPSLVILSGVLASHYIHIVKDVIRQQALSSVQDVDVVVSDLVDPALLGAASMVLDYTTRRIY

SEQ ID NO: 29 - DNA, 6217 bp, synthetic construct
  source 1..6217: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcgcccttatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagaagggcgaattcacgtgggcccggtaccgtatactctagagcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 30 - DNA, 6217 bp, synthetic construct
  source 1..6217: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcgaattcgcccttatggagaagaatggaaataaccgaaagctgcgggtttgtgttgctacttgtaaccgtgcagattattctaaacttgccccgatcatgtttggcattaaaaccgaacctgagttctttgaacttgatgttgtggtacttggctctcacctgatagatgactatggaaatacatatcgaatgattgaacaagatgactttgacattaacaccaggctacacacaattgtgaggggagaagatgaggcagccatggtggagtcagtaggcctggccctagtgaagctgccagatgtccttaatcgcctgaagcctgatatcatgattgttcatggagacaggtttgatgccctggctctggccacatctgctgccttgatgaacatccgaatccttcacattgaaggtggggaagtcagtgggaccattgatgactctatcagacatgccataacaaaactggctcattatcatgtgtgctgcacccgcagtgcagagcagcacctgatatccatgtgtgaggaccatgatcgcatccttttggcaggctgcccttcctatgacaaacttctctcagccaagaacaaagactacatgagcatcattcgcatgtggctaggtgatgatgtaaaatctaaagattacattgttgcactacagcaccctgtgaccactgacattaagcattccataaaaatgtttgaattaacattggatgcacttatctcatttaacaagcggaccctagtcctgtttccaaatattgacgcagggagcaaagagatggttcgagtgatgcggaagaagggcattgagcatcatcccaactttcgtgcagttaaacacgtcccatttgaccagtttatacagttggttgcccatgctggctgtatgattgggaacagcagctgtggggttcgagaagttggagcttttggaacacctgtgatcaacctgggaacacgtcagattggaagagaaacaggggagaatgttcttcatgtccgggatgctgacacccaagacaaaatattgcaagcactgcaccttcagtttggtaaacagtacccttgttcaaagatatatggggatggaaatgctgttccaaggattttgaagtttctcaaatctatcgatcttcaagagccactgcaaaagaaattctgctttcctcctgtgaaggagaatatctctcaagatattgaccatattcttgaaactctaagtgccttggccgttgatcttggcgggacgaacctccgagttgcaatagtcagcatgaagggtgaaatagttaagaagtatactcagttcaatcctaaaacctatgaagagaggattaatttaatcctacagatgtgtgtggaagctgcagcagaagctgtaaaactgaactgcagaattttgggagtaggcatttccacaggtggccgtgtaaatcctcgggaaggaattgtgctgcattcaaccaaactgatccaagagtggaactctgtggaccttaggacccccctttctgacactttgcatctccctgtgtgggtagacaatgatggcaactgtgctgccctggcggaaaggaaatttggccaaggaaagggactggaaaactttgttacacttatcacaggcacaggaatcggtggtggaattatccatcagcatgaattgatccacggaagctccttctgtgctgcagaactgggccaccttgttgtgtctctggatgggcctgattgttcctgtggaagccatgggtgcattgaagcatacgcctctggaatggccttgcagagggaggcaaaaaagctccatgatgaggacctgctcttggtggaagggatgtcagtgccaaaagatgaggctgtgggtgcgctccatctcatccaagctgcgaaacttggcaatgcgaaggcccagagcatcctaagaacagctggaacagctttgggtcttggggttgtgaacatcctccataccatgaatccctcccttgtgatcctctccggagtcctggccagtcactatatccacattgtcaaagacgtcattcgccagcaggccttgtcctccgtgcaggacgtggatgtggtggtttcggatttggttgaccccgccctgctgggtgctgccagcacggttctggactacacaacacgcaggatctactagaagggcgaattcacgtgggcccggtaccgtatactctagagcggccgcggatccagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctat

SEQ ID NO: 31 - DNA, 552 bp, synthetic construct
  source 1..552: mol_type = other DNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtcctgctgggtgctgccagcacggagatatgtgcatctccgtgctggcagcacccagcaggcattatggtgacagctgcctcgggaagccaagttgggctttaaagtgcagggcctgctgatgttgagtgctttttgttccatactgtgtatagccatcatcgagtgaagtagattagcatctccgtgctggcagcacccagcaggcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 32 - DNA, 552 bp, synthetic construct
  source 1..552: mol_type = other DNA
tttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgtctgctgggtgctgccagcacggtagatatgtgcatctaccgtgctggcagcacccagcagcattatggtgacagctgcctcgggaagccaagttgggctttaaagtgcagggcctgctgatgttgagtgctttttgttcatactgtgtgcgaccatcatcgtagtgaagtagattagcatctaccgtgctggcagcacccagcagcataagaagttatgtattcatccaataattcaagccaagcaagtatataggtgttttaatagtttttgttttggggattgtgaccagaagattttgaaaattaaatattactgaagatttcgacttccactgttaaatgtacaagatacatgaaatattaaagaaaatgtgtaactttttgtgtaaatacatcttgt

SEQ ID NO: 33-34 - skipped (2 entries)

SEQ ID NO: 35 - DNA, 69 bp, Homo sapiens
  source 1..69: mol_type = genomic DNA
ttggttgaccccgccctgctgggtgctgccagcatggttctggactacacaacacgcaggatctactag

SEQ ID NO: 36 - protein, 22 aa, Homo sapiens
LVDPALLGAASMVLDYTTRRIY

SEQ ID NO: 37 - DNA, 69 bp, Homo sapiens
  source 1..69: mol_type = genomic DNA
ttggttgaccccgccctgctgggtgctgccagcacggttctggactacacaacacgcaggatctactag

SEQ ID NO: 38 - protein, 22 aa, Homo sapiens
LVDPALLGAASTVLDYTTRRIY

SEQ ID NO: 39 - DNA, 29 bp, Homo sapiens
  source 1..29: mol_type = genomic DNA
tgaccccgccctgctgggtgccaccagca

SEQ ID NO: 40 - DNA, 39 bp, Homo sapiens
  source 1..39: mol_type = genomic DNA
tgaccccgccctgctgggtgccaccagcatggttctgga

SEQ ID NO: 41 - DNA, 39 bp, Homo sapiens
  source 1..39: mol_type = genomic DNA
tgaccccgccctgctgggtgccaccagcacggttctgga

SEQ ID NO: 42 - DNA, 532 bp, Homo sapiens
  source 1..532: mol_type = genomic DNA
ctccggagtcctggccagtcactatatccacattgtcaaagacgtcattcgccagcaggccttgtcctccgtgcaggacgtggatgtggtggtttcggatttggttgaccccgccctgctgggtgctgccagcatggttctggactacacaacacgcaggatctactagacctccaggaacagacatggaccttctctccagagctcctgagtggaatcaagttcttgtctttaggatgaccgtttcttaacaatcaaatctggtattgaactgcaggtgactttggcagagaaatgttttcacttttggtctcctcttccagagtcacctttccccactcctatttttgtagatgctattctttctgatgtcttcttactaggggtcattttagctcaaaccctgtaagttacagtcacaattttctgtgccaaagcagctacaataatagagaggaagccttcttagaactctgcttactaatgtattaataccactgagaccttcaggccttgcctgggatatcacttc